SEQUENCE LISTING (47 sequences)

SEQ ID NO: 1 - protein, 6 aa, Artificial Sequence
  synthetic peptide
XXGDFX

SEQ ID NO: 2 - protein, 6 aa, Artificial Sequence
  synthetic peptide
XXGDFV

SEQ ID NO: 3 - protein, 6 aa, Artificial Sequence
  synthetic peptide
GXGDFV

SEQ ID NO: 4 - protein, 6 aa, Artificial Sequence
  synthetic cyclic peptide
GXGDFV

SEQ ID NO: 5 - protein, 5 aa, Artificial Sequence
  synthetic cyclic peptide
RGDXV

SEQ ID NO: 6 - protein, 5 aa, Artificial Sequence
  synthetic cyclic peptide
RADXV

SEQ ID NO: 7 - protein, 5 aa, Artificial Sequence
  synthetic cyclic peptide
RGDFX

SEQ ID NO: 8 - protein, 15 aa, Artificial Sequence
  synthetic peptide
YTAECKPQVTRGDVF

SEQ ID NO: 9 - protein, 5 aa, Artificial Sequence
  synthetic cyclic peptide
RXDFV

SEQ ID NO: 10 - protein, 6 aa, Artificial Sequence
  synthetic cyclic peptide
ARGDXL

SEQ ID NO: 11 - protein, 6 aa, Artificial Sequence
  synthetic cyclic peptide
GRGDXL

SEQ ID NO: 12 - protein, 12 aa, Artificial Sequence
  synthetic  peptide
TRQVVCDLGNPM

SEQ ID NO: 13 - protein, 13 aa, Artificial Sequence
  synthetic  peptide
GVVRNNEALARLS

SEQ ID NO: 14 - protein, 11 aa, Artificial Sequence
  synthetic  peptide
TDVNGDGRHDL

SEQ ID NO: 15 - protein, 5 aa, Artificial Sequence
  synthetic  cyclic peptide
RGDXX

SEQ ID NO: 16 - protein, 5 aa, Artificial Sequence
  synthetic  cyclic peptide
RGEXX

SEQ ID NO: 17 - protein, 222 aa, Homo sapiens
KGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 18 - protein, 193 aa, Homo sapiens
ICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 19 - protein, 74 aa, Homo sapiens
ICKQDIVFDGIAQIRGEIFFFKDRFIWRTVTPRDKPMGPLLVATFWPELPEKIDAVYEAPQEEKAVFFAGNEYW

SEQ ID NO: 20 - protein, 108 aa, Homo sapiens
ICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWS

SEQ ID NO: 21 - protein, 122 aa, Homo sapiens
EYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 22 - protein, 89 aa, Homo sapiens
FNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 23 - protein, 228 aa, Gallus gallus
KGIQELYEVSPDVEPGPGPGPGPGPRPTLGPVTPELCKHDIVFDGVAQIRGEIFFFKDRFMWRTVNPRGKPTGPLLVATFWPDLPEKIDAVYESPQDEKAVFFAGNEYWVYTASNLDRGYPKKLTSLGLPPDVQRIDAAFNWGRNKKTYIFSGDRYWKYNEEKKKMELATPKFIADSWNGVPDNLDAVLGLTDSGYTYFFKDQYYLQMEDKSLKIVKIGKISSDWLGC

SEQ ID NO: 24 - protein, 193 aa, Gallus gallus
LCKHDIVFDGVAQIRGEIFFFKDRFMWRTVNPRGKPTGPLLVATFWPDLPEKIDAVYESPQDEKAVFFAGNEYWVYTASNLDRGYPKKLTSLGLPPDVQRIDAAFNWGRNKKTYIFSGDRYWKYNEEKKKMELATPKFIADSWNGVPDNLDAVLGLTDSGYTYFFKDQYYLQMEDKSLKIVKIGKISSDWLGC

SEQ ID NO: 25 - protein, 74 aa, Gallus gallus
LCKHDIVFDGVAQIRGEIFFFKDRFMWRTVNPRGKPTGPLLVATFWPDLPEKIDAVYESPQDEKAVFFAGNEYW

SEQ ID NO: 26 - protein, 108 aa, Gallus gallus
LCKHDIVFDGVAQIRGEIFFFKDRFMWRTVNPRGKPTGPLLVATFWPDLPEKIDAVYESPQDEKAVFFAGNEYWVYTASNLDRGYPKKLTSLGLPPDVQRIDAAFNWG

SEQ ID NO: 27 - protein, 122 aa, Gallus gallus
EYWVYTASNLDRGYPKKLTSLGLPPDVQRIDAAFNWGRNKKTYIFSGDRYWKYNEEKKKMELATPKFIADSWNGVPDNLDAVLGLTDSGYTYFFKDQYYLQMEDKSLKIVKIGKISSDWLGC

SEQ ID NO: 28 - protein, 89 aa, Gallus gallus
FNWGRNKKTYIFSGDRYWKYNEEKKKMELATPKFIADSWNGVPDNLDAVLGLTDSGYTYFFKDQYYLQMEDKSLKIVKIGKISSDWLGC

SEQ ID NO: 29 - DNA, 2123 bp, Gallus gallus
  CDS (132)...(2123)
aattccggcaaaagagaaaacggtgcagagagttaagatgtgcagataagcaactagtgcactgtgcagccaaagtaactgacagtcagtcagagaaatcttttaaagaggattgcaaaaatataggcagaatgaagactcacagtgtttttggcttcttttttaaagtactattaatccaagtgtatctttttaacaaaactttagctgcaccgtcaccaatcattaagttccctggagacagcactccaaaaacagacaaagagctagcagtgcaatacctgaataaatattatggatgcccaaaagacaattgcaacttatttgtattgaaagatactttgaagaaaatgcagaaattttttgggctgcctgaaacaggagatttggatcaaaacacaattgagacaatgaagaaaccccgctgtggtaaccccgatgtggccaattacaacttctttccaagaaagccaaaatgggaaaagaatcatataacatacaggattataggctataccccggatttggatcctgagacagtagatgatgcctttgcccgagcctttaaagtctggagtgatgtcacgccactgagatttaaccgaataaatgatggagaggcagacattatgattaattttggccgatgggaacatggtgatggctatccatttgatggcaaagatggtctcctggctcacgcctttgcaccggggccaggaattggaggagactcccattttgatgatgatgaactgtggactcttggagaagggcaagtggttagagtaaagtatggaaatgcagatggtgaatactgcaaatttcccttctggttcaatggtaaggaatacaacagctgcacagatgcaggacgtaatgatggattcctctggtgttccacaaccaaagactttgatgcagatggcaaatatggcttttgtccccatgagtcactttttacaatgggtggcaatggtgatggacagccctgcaagtttccctttaaatttcaaggccagtcctatgaccagtgtacaacagaaggcaggacagatggatacagatggtgtggaaccactgaagactatgatagagataagaaatacggattctgcccagaaactgccatgtcaacagttggtggaaattcagaaggagctccttgtgtattccccttcatcttccttgggaataaatacgactcctgtacaagtgcaggtcgcaatgatggcaagctgtggtgtgcttctaccagcagctatgatgatgaccgcaagtggggcttttgtccagatcaaggatacagtctcttcttggttgctgcccacgaatttggccatgcgatgggattagagcactccgaggacccaggagctctcatggccccgatctacacctacaccaagaacttccgcctttctcaggatgacattaaggggattcaggagctatatgaagtatcacctgatgtggaacctggaccagggccaggaccagggccaggaccacgtcctacccttggacctgtcactccagagctctgcaagcacgacattgtatttgatggagttgcacaaattagaggagaaatatttttcttcaaagacagattcatgtggaggactgtaaaccctcgaggaaaacccacaggtcctcttctcgttgctacattctggcctgatctgccagagaaaatcgatgctgtctacgagtcccctcaggatgagaaggctgtattttttgcaggaaatgagtactgggtttatacagccagcaacctggataggggctatccaaagaaactcaccagcctgggactaccccctgatgtgcaacgcattgatgcagccttcaactggggcagaaacaagaagacatatattttctctggagacagatactggaagtacaatgaagaaaagaaaaaaatggagcttgcaaccccaaaattcattgcggattcttggaatggagttccagataacctcgatgctgtcctgggtcttactgacagcgggtacacctattttttcaaagaccagtactatctacaaatggaagacaagagtttgaagattgttaaaattggcaagataagttctgactggttgggttgctga

SEQ ID NO: 30 - protein, 663 aa, Gallus gallus
MKTHSVFGFFFKVLLIQVYLFNKTLAAPSPIIKFPGDSTPKTDKELAVQYLNKYYGCPKDNCNLFVLKDTLKKMQKFFGLPETGDLDQNTIETMKKPRCGNPDVANYNFFPRKPKWEKNHITYRIIGYTPDLDPETVDDAFARAFKVWSDVTPLRFNRINDGEADIMINFGRWEHGDGYPFDGKDGLLAHAFAPGPGIGGDSHFDDDELWTLGEGQVVRVKYGNADGEYCKFPFWFNGKEYNSCTDAGRNDGFLWCSTTKDFDADGKYGFCPHESLFTMGGNGDGQPCKFPFKFQGQSYDQCTTEGRTDGYRWCGTTEDYDRDKKYGFCPETAMSTVGGNSEGAPCVFPFIFLGNKYDSCTSAGRNDGKLWCASTSSYDDDRKWGFCPDQGYSLFLVAAHEFGHAMGLEHSEDPGALMAPIYTYTKNFRLSQDDIKGIQELYEVSPDVEPGPGPGPGPGPRPTLGPVTPELCKHDIVFDGVAQIRGEIFFFKDRFMWRTVNPRGKPTGPLLVATFWPDLPEKIDAVYESPQDEKAVFFAGNEYWVYTASNLDRGYPKKLTSLGLPPDVQRIDAAFNWGRNKKTYIFSGDRYWKYNEEKKKMELATPKFIADSWNGVPDNLDAVLGLTDSGYTYFFKDQYYLQMEDKSLKIVKIGKISSDWLGC

SEQ ID NO: 31 - DNA, 21 bp, Artificial Sequence
  PCR primer
attgaattcttctacagttca

SEQ ID NO: 32 - DNA, 21 bp, Artificial Sequence
  PCR primer
atgggatccactgcaaatttc

SEQ ID NO: 33 - DNA, 21 bp, Artificial Sequence
  PCR primer
gccggatccatgaccagtgta

SEQ ID NO: 34 - DNA, 21 bp, Artificial Sequence
  PCR primer
gtgggatccctgaagactatg

SEQ ID NO: 35 - DNA, 21 bp, Artificial Sequence
  PCR primer
aggggatccttaaggggattc

SEQ ID NO: 36 - DNA, 21 bp, Artificial Sequence
  PCR primer
ctcggatcctctgcaagcacg

SEQ ID NO: 37 - DNA, 21 bp, Artificial Sequence
  PCR primer
ctcggatcctctgcaagcacg

SEQ ID NO: 38 - DNA, 26 bp, Artificial Sequence
  PCR primer
gcaggatccgagtgctgggtttatac

SEQ ID NO: 39 - DNA, 27 bp, Artificial Sequence
  PCR primer
gcagaattcaactgtggcagaaacaag

SEQ ID NO: 40 - DNA, 26 bp, Artificial Sequence
  PCR primer
gtagaattccagcactcatttcctgc

SEQ ID NO: 41 - DNA, 24 bp, Artificial Sequence
  PCR primer
tctgaattctgccacagttgaagg

SEQ ID NO: 42 - DNA, 21 bp, Artificial Sequence
  PCR primer
attgaattcttctacagttca

SEQ ID NO: 43 - DNA, 20 bp, Artificial Sequence
  PCR primer
gatgaattctactgcaagtt

SEQ ID NO: 44 - DNA, 21 bp, Artificial Sequence
  PCR primer
cactgaattcatctgcaaaca

SEQ ID NO: 45 - protein, 429 aa, Homo sapiens
YCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 46 - protein, 5 aa, Artificial Sequence
  synthetic peptide
XGXXX

SEQ ID NO: 47 - protein, 5 aa, Artificial Sequence
  synthetic peptide
XGXXX

(19) United States
(12) Patent Application Publication
(10) Pub. No.: US 2004/0063790 A1
(43) Pub. Date: Apr. 1, 2004

(54) Methods for inhibition of angiogenesis

(75) Inventors: Peter C. Brooks, Carmel, NY (US); David A. Cheresh, Encinitas, CA (US)

Correspondence Address: OLSON & HIERL, LTD., 36th Floor, 20 North Wacker Drive, Chicago, IL, 60606, US

(73) Assignee: The Scripps Research Institute

(21) Appl. No.: 10/402,212

(22) Filed: Mar. 28, 2003

Related U.S. Application Data
(63) Continuation-in-part of application No. 10/115,223, filed on Apr. 2, 2002.
Application No. 10/115,223 is a continuation of application No. 09/194,468, filed on Mar. 23, 1999, now Pat. No. 6,500,924.
Application No. 09/194,468 is a 371 of international application No. PCT/US97/09158, filed on May 30, 1997.
(60) Provisional application No. 60/018,773, filed on May 31, 1996.
(60) Provisional application No. 60/015,869, filed on May 31, 1996.

(51) Int. Cl. A61K 31/195

(52) U.S. Cl. 514/563; 514/567

(57) ABSTRACT

The present invention describes methods for inhibition angiogenesis in tissues using organic peptidomimetic αᵥβ₃ antagonists, and particularly for inhibiting angiogenesis in inflamed tissues and in tumor tissues and metastases using therapeutic compositions containing αᵥβ₃ antagonists. The antagonists are organic compounds having a basic group and an acidic group spaced from one another by a distance in the range of about 10 Angstroms to about 100 Angstroms, as described in detail herein.

### CROSS-REFERENCE TO RELATED APPLICATIONS

This application is a continuation-in-part of U.S. application for patent Ser. No. 10/115,223, filed on Apr. 2, 2002, which is a continuation of U.S. application for patent Ser. No. 09/194,468, filed on Mar. 23, 1999, now U.S. Pat. No. 6,500,924, which is a U.S. National Phase application of PCT/US97/09158, filed on May 30, 1997, which claims priority from U.S. Provisional Application Serial No. 60/018,773, filed on May 31, 1996 and U.S. Provisional Application Serial No. 60/015,869, filed on May 31, 1996, the disclosures of which are incorporated herein by reference.

### STATEMENT OF GOVERNMENT INTERESTS

This invention was made with government support under Contract Nos. CA50826, CA45726, HL54444, T32 AI07244-11 and F32 CA72192 by the National Institutes of Health. The government has certain rights in the invention.

### TECHNICAL FIELD

The present invention relates generally to the field of medicine, and relates specifically to methods and compositions for inhibiting angiogenesis of tissues using antagonists to the vitronectin receptor αᵥβ₃.

### BACKGROUND OF THE INVENTION

Integrins are a class of cellular receptors known to bind extracellular matrix proteins, and therefore mediate cell-cell and cell-extracellular matrix interactions, referred generally to as cell adhesion events. However, although many integrins and the ligands that bind an integrin are described in the literature, the biological function of many of the integrins remains elusive. The integrin receptors constitute a family of proteins with shared structural characteristics of noncovalent heterodimeric glycoprotein complexes formed of α and β subunits.

The vitronectin receptor, named for its original characteristic of preferential binding to vitronectin, is now known to refer to three different integrins, designated αᵥβ₁, αᵥβ₃ and αᵥβ₅. Horton, Int. J. Exp. Pathol., 71:741-759 (1990). αᵥβ₁ binds fibronectin and vitronectin. αᵥβ₃ binds a large variety of ligands, including fibrin, fibrinogen, laminin, thrombospondin, vitronectin, von Willebrand's factor, osteospontin and bone sialoprotein I. αᵥβ₅ binds vitronectin. The specific cell adhesion roles these three integrins play in the many cellular interactions in tissues are still under investigation, but it is clear that there are different integrins with different biological functions.

One important recognition site in the ligand for many integrins is the arginine-glycine-aspartic acid (RGD) tripeptide sequence. RGD is found in all of the ligands identified above for the vitronectin receptor integrins. This RGD recognition site can be mimicked by polypeptides (“peptides”) that contain the RGD sequence, and such RGD peptides are known inhibitors of integrin function. It is important to note, however, that depending upon the sequence and structure of the RGD peptide, the specificity of the inhibition can be altered to target specific integrins.

For discussions of the RGD recognition site, see Pierschbacher et al., Nature, 309:30-33 (1984), and Pierschbacher et al., Proc. Natl. Acad. Sci., USA, 81:5985-5988 (1984). Various RGD polypeptides of varying integrin specificity have also been described by Grant et al., Cell, 58:933-943 (1989), Cheresh et al., Cell, 58:945-953 (1989), Aumailley et al., FEBS Letts., 291:50-54 (1991), and Pfaff et al., J. Biol. Chem., 269:20233-20238 (1994), and in U.S. Pat. Nos. 4,517,686, 4,578,079, 4,589,881, 4,614,517, 4,661,111, 4,792,525, 4,683,291, 4,879,237, 4,988,621, 5,041,380 and 5,061,693.

Angiogenesis is a process of tissue vascularization that involves the growth of new developing blood vessels into a tissue, and is also referred to as neo-vascularization. The process is mediated by the infiltration of endothelial cells and smooth muscle cells. The process is believed to proceed in any one of three ways: the vessels can sprout from pre-existing vessels, de-novo development of vessels can arise from precursor cells (vasculogenesis), or existing small vessels can enlarge in diameter. Blood et al., Bioch. Biophys. Acta, 1032:89-118 (1990). Vascular endothelial cells are known to contain at least five RGD-dependent integrins, including the vitronectin receptor (αᵥβ₃ or αᵥβ₅), the collagen Types I and IV receptor (αᵥβ₁) the laminin receptor (αᵥβ₁), the fibronectin/laminin/collagen receptor (αᵥβ₁) and the fibronectin receptor (αᵥβ₁). Davis et al., J. Cell. Biochem., 51:206-218 (1993). The smooth muscle cell is known to contain at least six RGD-dependent integrins, including αᵥβ₁, αᵥβ₃ and αᵥβ₅.

Angiogenesis is an important process in neonatal growth, but is also important in wound healing and in the pathogenesis of a large variety of clinical diseases including tissue inflammation, arthritis, tumor growth, diabetic retinopathy, macular degeneration by neovascularization of retina and the like conditions. These clinical manifestations associated with angiogenesis are referred to as angiogenic diseases. Folkman et al., Science, 235:442-447 (1987). Angiogenesis is generally absent in adult or mature tissues, although it does occur in wound healing and in the corpeus leuteum growth cycle. See, for example, Moses et al., Science, 248:1408-1410 (1990).

It has been proposed that inhibition of angiogenesis would be a useful therapy for restricting tumor growth. Inhibition of angiogenesis has been proposed by (1) inhibition of release of “angiogenic molecules” such as bFGF (basic fibroblast growth factor), (2) neutralization of angiogenic molecules, such as by use of anti-βbFGF antibodies, and (3) inhibition of endothelial cell response to angiogenic stimuli. This latter strategy has received attention, and Folkman et al., Cancer Biology, 3:89-96 (1992), have described several endothelial cell response inhibitors, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D₃ analogs, alpha-interferon, and the like that might be used to inhibit angiogenesis. For additional proposed inhibitors of angiogenesis, see Blood et al., Bioch. Biophys. Acta., 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, and 5,202,352. None of the inhibitors of angiogenesis described in the foregoing references are targeted at inhibition of αᵥβ₃.

RGD-containing peptides that inhibit vitronectin receptor αᵥβ₃ have also been described. Aumailley et al., FEBS Letts., 291:50-54 (1991), Choi et al., J. Vasc. Surg., 19:125-134 (1994), Smith et al., J. Biol. Chem., 265:12267-12271 (1990), and Pfaff et al., J. Biol. Chem., 269:20233-20238 (1994). However, the role of the integrin αᵥβ₃ in angiogenesis has never been suggested nor identified until the present invention.

For example, Hammes et al., Nature Med., 2:529-53 (1996) confirmed the findings of the present invention. Specifically, the paper shows that cyclic peptides including cyclic RGDfV, the structure and function of the latter of which has been previously described in the priority applications on which the present application is based, inhibited retinal neovascularization in a mouse model of hypoxia-induced retinal neovascularization. In a separate study that also supports the present invention as well as the priority applications, Luna et al., Lab. Invest., 75:563-573 (1996) described two particular cyclic methylated RGD-containing peptides that were partially effective at inhibiting retinal neovascularization in the mouse model of oxygen-induced ischemic retinopathy. In contrast, the peptides of the present invention exhibit almost complete inhibition of neovascularization in the model systems described herein.

Inhibition of cell adhesion in vitro using monoclonal antibodies immunospecific for various integrin α or β subunits have implicated αᵥβ₃ in cell adhesion of a variety of cell types including microvascular endothelial cells. Davis et al., J. Cell. Biol., 51:206-218 (1993). In addition, Nicosia et al., Am. J. Pathol., 138:829-833 (1991), described the use of the RGD peptide GRGDS to in vitro inhibit the formation of “microvessels” from rat aorta cultured in collagen gel. However, the inhibition of formation of “microvessels” in vitro in collagen gel cultures is not a model for inhibition of angiogenesis in a tissue because it is not shown that the microvessel structures are the same as capillary sprouts or that the formation of the microvessel in collagen gel culture is the same as neovascular growth into an intact tissue, such as arthritic tissue, tumor tissue or disease tissue where inhibition of angiogenesis is desirable.

For angiogenesis to occur, endothelial cells must first degrade and cross the blood vessel basement membrane in a similar manner used by tumor cells during invasion and metastasis formation.

The inventors have previously reported that angiogenesis depends on the interaction between vascular integrins and extracellular matrix proteins. Brooks et al., Science, 264:569-571 (1994). Furthermore, it was reported that programmed cell death (apoptosis) of angiogenic vascular cells is initiated by the interaction, which would be inhibitied by certain antagonists of the vascular integrin αᵥβ₃. Brooks et al., Cell, 79:1157-1164 (1994). More recently, the inventors have reported that the binding of matrix metalloproteinase-2 (MMP-2) to vitronectin receptor (αᵥβ₅) can be inhibited using αᵥβ₅ antagonists, and thereby inhibit the enzymatic function of the proteinase. Brooks et al., Cell, 85:683-693 (1996).

Other than the studies reported here, Applicants are unaware of any other demonstration that angiogenesis could be inhibited in a tissue using inhibitors of cell adhesion. In particular, it has never been previously demonstrated by others that αᵥβ₃ function is required for angiogenesis in a tissue or that αᵥβ₃ antagonists can inhibit angiogenesis in a tissue.

### BRIEF DESCRIPTION OF THE INVENTION

The present invention disclosure demonstrates that angiogenesis in tissues requires integrin αᵥβ₃ and that inhibitors of αᵥβ₃ can inhibit angiogenesis. The disclosure also demonstrates that antagonists of other integrins, such as αᵥβ₃, or αᵥβ₁, do not inhibit angiogenesis, presumably because these other integrins are not essential for angiogenesis to occur.

The invention therefore describes methods for inhibiting angiogenesis in a tissue comprising administering to the tissue a composition comprising an angiogenesis-inhibiting amount of an αᵥβ₃ antagonist.

The tissue to be treated can be any tissue in which inhibition of angiogenesis is desirable, such as diseased tissue where neo-vascularization is occurring. Exemplary tissues include inflamed tissue, solid tumors, metastases, tissues undergoing restenosis, and the like tissues.

An αᵥβ₃ antagonist for use in the present methods is capable of binding to αᵥβ₃ and competitively inhibiting the ability of αᵥβ₃ to bind to a natural ligand. Preferably, the antagonist exhibits specificity for αᵥβ₃ over other integrins. In a preferred embodiment, the αᵥβ₃ antagonist inhibits binding of fibrinogen or other RGD-containing ligands to αᵥβ₃ but does not substantially inhibit binding of fibrinogen to α_{IIb}β₃. A preferred αᵥβ₃ antagonist can be a cyclic or linear polypeptide, an organic αᵥβ₃ antagonist (e.g., an organic peptidomimetic αᵥβ₃ antagonist), or functional fragment thereof.

Most preferably the organic αᵥβ₃ antagonist is an organic peptidomimetic compound having a basic group and an acidic group spaced from one another by a distance in the range of about 10 Angstroms to about 100 Angstroms.

Preferred organic peptidomimetic αᵥβ₃ antagonist compounds having a basic group and an acidic group spaced from one another by a distance in the range of about 10 Angstroms to about 100 Angstroms that are useful in the methods of the present invention have the following general formula (I):

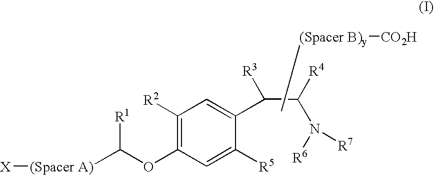

wherein R¹ and R² are both H or together form a radical selected from the group consisting of —CH₂—C(O)—, ═C—C(O)—, and —C(O)—NH—;

R³ and R⁴ are both H or together form a covalent bond;

R⁵ and R⁶ are both H or, when R³ and R⁴ together form a covalent bond, R⁵ and R⁶ together form a covalent bond;

R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;

X is selected from the group consisting of 2-imidazolyl, 2-benzimidazolyl, N-guanidyl, N—(C₁-C₂)alkyl-substituted guanidyl, 2-pyridyl, 4-carbonimidophenyl, and 6-(2-methylaminopyridyl);

Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O)CH₂—, and —NHC(O)CH₂CH₂—;

Spacer B is a radical selected from the group consisting of —CH₂—, —CH₂CH₂—, and —CH(R⁸)CH₂—;

Z¹ and Z² are both covalent bonds to a bridging carbonyl group forming a cyclic urethane;

R⁸ is phenyl or 5-benzo-2, 1, 3-thiadiazolyl; and

Spacer B is covalently bonded to either of the carbon atoms bearing substituents R³ and R⁴; y is 0 or 1, with the proviso that when R⁵ and R⁶ form a covalent bond, y is 1.

### BRIEF DESCRIPTION OF THE DRAWINGS

In the drawings forming a portion of this disclosure:

FIG. 1 illustrates the quantification in a bar graph of the relative expression of αᵥβ₃ and β₁ in untreated and bFGF treated 10 day old CAMs. The mean fluorescence intensity is plotted on the Y-axis with the integrin profiles plotted on the X-axis.

FIG. 2 illustrates the quantification of the number of vessels entering a tumor in a CAM preparation. The graph shows the number of vessels as plotted on the Y-axis resulting from topical application of either CSAT (anti-β₁), LM609 (anti-αᵥβ₃) or P3G2 (anti-αᵥβ₅).

FIGS. 3A-3D illustrate a comparison between wet tumor weights 7 days following treatment and initial tumor weights. Each bar represents the mean ±S.E. of 5-10 tumors per group. Tumors were derived from human melanoma (M21-L) (FIG. 3A), pancreatic carcinoma (Fg) (FIG. 3B), lung carcinoma (UCLAP-3) (FIG. 3C), and laryngeal carcinoma (HEp3) (FIG. 3D) CAM preparations and treated intravenously with PBS, CSAT (anti-β₁), or LM609 (anti-αᵥβ₃). The graphs show the tumor weight as plotted on the Y-axis resulting from intravenous application of either CSAT (anti-β₁), LM609 (anti-αᵥβ₃) or PBS as indicated on the X-axis.

FIG. 4 represents a flow chart of how the in vivo mouse:human chimeric mouse model was generated. A portion of skin from a SCID mouse was replaced with human neonatal foreskin and allowed to heal for 4 weeks. After the graft had healed, the human foreskin was inoculated with human tumor cells. During the following 4 week period, a measurable tumor was established which comprised a human tumor with human vasculature growing from the human skin into the human tumor.

FIG. 5 illustrates the percent of apoptosis of cells derived from mab-treated and peptide-treated CAMs and stained with Apop Tag as determined by FACS analysis. The striped and stippled bars represent cells from embryos treated 24 hours and 48 hours prior to the assay, respectively. Each bar represents the mean ±S.E. of three replicates. CAMs were treated mAb LM609 (anti-αᵥβ₃), or CSAT (anti-β₁), or PBS. CAMs were also treated with cyclic peptide 66203 (cyclo-RGDfV, indicated as Peptide 203) or control cyclic peptide 69601 (cyclo-PADfV, indicated as Peptide 601).

FIG. 6 shows the result of a inhibition of cell attachment assay with peptide 85189. The effects of the peptide antagonist was assessed over a dosage range of 0.001 to 100 uM as plotted on the X-axis. Cell attachment is plotted on the Y-axis measured at an optical density (O.D.) of 600 nm. Cell attachment was measured on vitronectin-(broken lines) versus laminin-(solid lines) coated surfaces.

FIGS. 7A-7D show the consecutive cDNA sequence of chicken MMP-2 along with the deduced amino acid sequence shown on the second line. The third and fourth lines respectively show the deduced amino acid sequence of human and mouse MMP-2. The chicken cDNA sequence is listed in SEQ ID NO: 29 along with the encoded amino acid sequence that is also presented separately as SEQ ID NO: 30. The numbering of the first nucleotide of the 5′ untranslated region and the region encoding the proenzyme sequence shown in FIG. 7A as a negative number is actually presented as number 1 in Sequence Listing making the latter appear longer than the FIGS.; however, the nucleotide sequence is the FIGS. is identical in length and sequence to that as presented in the listing with the exception of the numbering. Accordingly, references to nucleotide position for chicken or human MMP-2 in the specification, such as in primers for use in amplifying MMP-2 fragments, are based on the nucleotide position as indicated in the FIGS. and not as listed in the Sequence Listing.

FIG. 8 shows the results in bar-graph form of a solid-phase receptor binding assay of iodinated MMP-2 to bind to αᵥβ₃ with and without the presence of inhibitors. The data is plotted as bound CPM on the Y-axis against the various potential inhibitors and controls.

FIG. 9 shows the specificity of chicken-derived MMP-2 compositions for either the integrin receptors αᵥβ₃ and α_{IIb}β₃ in the presence of MMP-2 inhibitors.

FIGS. 10 and 11 both illustrate in bar graph form the angiogenic index (a measurement of branch points) of the effects of chicken MMP-2(410-637) GST fusion protein (labeled CTMMP-2) versus control (RAP-GST or GST-PAP) on bFGF-treated CAMs. Angiogenic index is plotted on the Y-axis against the separate treatments on the X-axis.

FIG. 12 shows the effects of peptides and organic compounds on bFGF-induced angiogenesis as measured by the effect on branch points plotted on the Y-axis against the various treatments on the X-axis, including bFGF alone, and bFGF-treated CAMs with peptides 69601 or 66203 and organic componds 96112, 96113 and 96229.

FIG. 13 graphically shows the dose response of peptide 85189 on inhibiting bFGF-induced angiogenesis where the number of branch points are plotted on the Y-axis against the amount of peptide administered to the embryo on the X-axis.

FIG. 14 shows the inhibitory activity of peptides 66203 (labeled 203) and 85189 (labeled 189) in bFGF-induced angiogenesis in the CAM assay. Controls included no peptide in bFGF-treated CAMS and peptide 69601 (labeled 601).

FIGS. 15, 16 and 17 respectively show the reduction in tumor weight for UCLAP-3, M21-L and FgM tumors following intravenous exposure to control peptide 69601 and antagonist 85189. The data is plotted with tumor weight on the Y-axis against the peptide treatments on the X-axis.

FIG. 18 illustrates the effect of peptides and antibodies on melanoma tumor growth in the chimeric mouse:human model. The peptides assessed included control 69601 (labeled 601) and antagonist 85189 (labeled 189). The antibody tested was LM609. Tumor volume in mm³ is plotted on the Y-axis against the various treatments on the X-axis.

FIGS. 19A and 19B respectively show the effect of antagonist 85189 (labeled 189) compared to control peptide 69601 (labeled 601) in reducing the volume and wet weight of M21L tumors over a dosage range of 10, 50 and 250 μg/injection.

FIGS. 20A and 20B show the effectiveness of antagonist peptide 85189 (labeled 189 with a solid line and filled circles) against control peptide 69601 (labeled 601 on a dotted line and open squares) at inhibiting M21L tumor volume in the mouse:human model with two different treatment regimens. Tumor volume in mm³ is plotted on the Y-axis against days on the X-axis.

FIGS. 21 through 25 schematically illustrate the various chemical syntheses of organic molecule αᵥβ₃ antagonists.

FIGS. 26 and 27 show the effects of various organic molecules on bFGF-induced angiogenesis in a CAM assay. Branch points are plotted on the Y-axis against the various compounds used at 250 μg/ml on the X-axis in FIG. 26 and 100 μg/ml in FIG. 27.

FIGS. 28 through 31 illustrate examples of organic peptidomimetic Compounds I(a) through I(r), corresponding to general formula (I), which are useful in the methods of the present invention.

FIG. 32 graphically illustrates the inhibitory effect of Compound I(e) of the invention in chick CAM angiogenesis inhibition assay.

FIG. 33 graphically depicts the inhibitory effect of Compound I(f) of the invention in a chick CAM angiogenesis inhibition assay.

FIG. 34 graphically illustrates the effects of Compound I(d) on M21-L tumor growth in athymic Wehi mice at concentrations of Compound I(d) ranging from about 3 mg/Kg/day to about 90 mg/Kg/day.

### DETAILED DESCRIPTION OF PREFERRED EMBODIMENTS

A. Definitions

Amino Acid Residue: An amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide.

The following acronyms have the meanings below:

| | |
|---|---|
| BOC | tert-butoxycarbonyl |
| DCCI | dicylcohexylcarbodiimide |
| DMF | dimethylformamide |
| OMe | methoxy |
| HOBt | 1-hydroxybenzotriazole |
| Fmoc | 9-fluorenylmethoxycarbonyl |
| Mtr | 2,3,6-trimethyl-4-methoxybenzenesulfonyl |

It should be noted that all amino acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino acid residues.

Polypeptide: refers to a linear series of amino acid residues connected to one another by peptide bonds between the alpha-amino group and carboxy group of contiguous amino acid residues.

Peptide: as used herein refers to a linear series of no more than about 50 amino acid residues connected one to the other as in a polypeptide.

Cyclic peptide: refers to a compound having a heteroatom ring structure that includes several amide bonds as in a typical peptide. The cyclic peptide can be a “head to tail” cyclized linear polypeptide in which a linear peptide's N-terminus has formed an amide bond with the terminal carboxylate of the linear peptide, or it can contain a ring structure in which the polymer is homodetic or heterodetic and comprises amide bonds and/or other bonds to close the ring, such as disulfide bridges, thioesters, thioamides, guanidino, and the like linkages.

Protein: refers to a linear series of greater than 50 amino acid residues connected one to the other as in a polypeptide.

Fusion protein: refers to a polypeptide containing at least two different polypeptide domains operatively linked by a typical peptide bond (“fused”), where the two domains correspond to peptides not found fused in nature.

Synthetic peptide: refers to a chemically produced chain of amino acid residues linked together by peptide bonds that is free of naturally occurring proteins and fragments thereof.

B. General Considerations

The present invention relates generally to the discovery that angiogenesis is mediated by the specific vitronectin receptor αᵥβ₃, and that inhibition of αᵥβ₃ function inhibits angiogenesis. This discovery is important because of the role that angiogenesis plays in a variety of disease processes. By inhibiting angiogenesis, one can intervene in the disease, ameliorate the symptoms, and in some cases cure the disease.

Where the growth of new blood vessels is the cause of, or contributes to, the pathology associated with a disease, inhibition of angiogenesis will reduce the deleterious effects of the disease. Examples include rheumatoid arthritis, diabetic retinopathy, inflammatory diseases, restenosis, and the like. Where the growth of new blood vessels is required to support growth of a deleterious tissue, inhibition of angiogenesis will reduce the blood supply to the tissue and thereby contribute to reduction in tissue mass based on blood supply requirements. Examples include growth of tumors where neovascularization is a continual requirement in order that the tumor grow beyond a few millimeters in thickness, and for the establishment of solid tumor metastases.

The methods of the present invention are effective in part because the therapy is highly selective for angiogenesis and not other biological processes. As shown in the Examples, only new vessel growth contains substantial αᵥβ₃, and therefore the therapeutic methods do not adversely effect mature vessels. Furthermore, αᵥβ₃ is not widely distributed in normal tissues, but rather is found selectively on new vessels, thereby assuring that the therapy can be selectively targeted to new vessel growth.

The discovery that inhibition of αᵥβ₃ alone will effectively inhibit angiogenesis allows for the development of therapeutic compositions with potentially high specificity, and therefore relatively low toxicity. Thus although the invention discloses the use of peptide-based reagents which have the ability to inhibit one or more integrins, one can design other reagents which more selectively inhibit αᵥβ₃, and therefore do not have the side effect of inhibiting other biological processes other that those mediated by αᵥβ₃.

For example, as shown by the present teachings, it is possible to prepare monoclonal antibodies highly selective for immunoreaction with αᵥβ₃ that are similarly selective for inhibition of αᵥβ₃ function. In addition, RGD-containing peptides can be designed to be selective for inhibition of αᵥβ₃, as described further herein.

Prior to the discoveries of the present invention, it was not known that angiogenesis, and any of the processes dependent on angiogenesis, could be inhibited in vivo by the use of reagents that antagonize the biological function of αᵥβ₃.

C. Methods for Inhibition of Angiogenesis

The invention provides for a method for the inhibition of angiogenesis in a tissue, and thereby inhibiting events in the tissue which depend upon angiogenesis. Generally, the method comprises administering to the tissue a composition comprising an angiogenesis-inhibiting amount of an αᵥβ₃ antagonist.

As described earlier, angiogenesis includes a variety of processes involving neovascularization of a tissue including “sprouting”, vasculogenesis, or vessel enlargement, all of which angiogenesis processes are mediated by and dependent upon the expression of αᵥβ₃. With the exception of traumatic wound healing, corpus leuteum formation and embryogenesis, it is believed that the majority of angiogenesis processes are associated with disease processes and therefore the use of the present therapeutic methods are selective for the disease and do not have deleterious side effects.

There are a variety of diseases in which angiogenesis is believed to be important, referred to as angiogenic diseases, including but not limited to, inflammatory disorders such as immune and non-immune inflammation, chronic rheumatoid arthritis and psoriasis, disorders associated with inappropriate or inopportune invasion of vessels such as diabetic retinopathy, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

Thus, methods which inhibit angiogenesis in a diseased tissue ameliorates symptoms of the disease and, depending upon the disease, can contribute to cure of the disease. In one embodiment, the invention contemplates inhibition of angiogenesis, per se, in a tissue. The extent of angiogenesis in a tissue, and therefore the extent of inhibition achieved by the present methods, can be evaluated by a variety of methods, such as are described in the Examples for detecting αᵥβ₃-immunopositive immature and nascent vessel structures by immunohistochemistry.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli.

Thus, in one related embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

The patient treated in the present invention in its many embodiments is desirably a human patient, although it is to be understood that the principles of the invention indicate that the invention is effective with respect to all mammals, which are intended to be included in the term “patient”. In this context, a mammal is understood to include any mammalian species in which treatment of diseases associated with angiogenesis is desirable, particularly agricultural and domestic mammalian species.

In another related embodiment, a tissue to be treated is a retinal tissue of a patient with a retinal disease such as diabetic retinopathy, macular degeneration or neovascular glaucoma and the angiogenesis to be inhibited is retinal tissue angiogenesis where there is neovascularization of retinal tissue.

In an additional related embodiment, a tissue to be treated is a tumor tissue of a patient with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include tumors of the lung, pancreas, breast, colon, laryngeal, ovarian, and the like tissues. Exemplary tumor tissue angiogenesis, and inhibition thereof, is described in the Examples.

Inhibition of tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

In a related embodiment, the invention contemplates the practice of the method in conjunction with other therapies such as conventional chemotherapy directed against solid tumors and for control of establishment of metastases. The administration of angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

Insofar as the present methods apply to inhibition of tumor neovascularization, the methods can also apply to inhibition of tumor tissue growth, to inhibition of tumor metastases formation, and to regression of established tumors. The Examples demonstrate regression of an established tumor following a single intravenous administration of an αᵥβ₃ antagonist of this invention.

Restenosis is a process of smooth muscle cell (SMC) migration and proliferation at the site of percutaneous transluminal coronary angioplasty which hampers the success of angioplasty. The migration and proliferation of SMC's during restenosis can be considered a process of angiogenesis which is inhibited by the present methods. Therefore, the invention also contemplates inhibition of restenosis by inhibiting angiogenesis according to the present methods in a patient following angioplasty procedures. For inhibition of restenosis, the αᵥβ₃ antagonist is typically administered after the angioplasty procedure for from about 2 to about 28 days, and more typically for about the first 14 days following the procedure.

The present method for inhibiting angiogenesis in a tissue, and therefore for also practicing the methods for treatment of angiogenesis-related diseases, comprises contacting a tissue in which angiogenesis is occurring, or is at risk for occurring, with a composition comprising a therapeutically effective amount of an αᵥβ₃ antagonist capable of inhibiting αᵥβ₃ binding to its natural ligand. Thus the method comprises administering to a patient a therapeutically effective amount of a physiologically tolerable composition containing an αᵥβ₃ antagonist of the invention.

The dosage ranges for the administration of the αᵥβ₃ antagonist depend upon the form of the antagonist, and its potency, as described further herein, and are amounts large enough to produce the desired effect in which angiogenesis and the disease symptoms mediated by angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of αᵥβ₃ antagonist sufficient to produce a measurable inhibition of angiogenesis in the tissue being treated, i.e., an angiogenesis-inhibiting amount. Inhibition of angiogenesis can be measured in situ by immunohistochemistry, as described herein, or by other methods known to one skilled in the art.

Insofar as an αᵥβ₃ antagonist can take the form of a αᵥβ₃ mimetic, an RGD-containing peptide, or a fragment thereof, it is to be appreciated that the potency, and therefore an expression of a “therapeutically effective” amount can vary. However, as shown by the present assay methods, one skilled in the art can readily assess the potency of a candidate αᵥβ₃ antagonist of this invention.

Potency of an αᵥβ₃ antagonist can be measured by a variety of means including inhibition of angiogenesis in the CAM assay, in the in vivo rabbit eye assay, in the in vivo chimeric mouse:human assay, and by measuring inhibition of binding of natural ligand to αᵥβ₃, all as described herein, and the like assays.

A preferred αᵥβ₃ antagonist has the ability to substantially inhibit binding of a natural ligand such as fibrinogen or vitronectin to αᵥβ₃ in solution at antagonist concentrations of less than 0.5 micromolar (μm), preferably less than 0.1 μm, and more preferably less than 0.05 μm. By “substantially” is meant that at least a 50 percent reduction in binding of fibrinogen is observed by inhibition in the presence of the αᵥβ₃ antagonist, and at 50% inhibition is referred to herein as an IC₅₀ value.

A more preferred αᵥβ₃ antagonist exhibits selectivity for αᵥβ₃ over other integrins. Thus, a preferred αᵥβ₃ antagonist substantially inhibits fibrinogen binding to αᵥβ₃ but does not substantially inhibit binding of fibrinogen to another integrin, such as αᵥβ₁, αᵥβ₅ or α_{II}β₃. Particularly preferred is an αᵥβ₃ antagonist that exhibits a 10-fold to 100-fold lower IC₅₀ activity at inhibiting fibrinogen binding to αᵥβ₃ compared to the IC₅₀ activity at inhibiting fibrinogen binding to another integrin. Exemplary assays for measuring IC₅₀ activity at inhibiting fibrinogen binding to an integrin are described in the Examples.

A therapeutically effective amount of an αᵥβ₃ antagonist of this invention in the form of a monoclonal antibody is typically an amount such that when administered in a physiologically tolerable composition is sufficient to achieve a plasma concentration of from about 0.01 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

A therapeutically effective amount of an αᵥβ₃ antagonist of this invention in the form of a polypeptide, or other similarly-sized small molecule αᵥβ₃ peptidomimetic, is typically an amount of polypeptide or peptidomimetic such that when administered in a physiologically tolerable composition is sufficient to achieve a plasma concentration of from about 0.1 microgram (μg) per milliliter (ml) to about 200 μg/ml, preferably from about 1 μg/ml to about 150 μg/ml. Based on a polypeptide or peptidomimetic having a mass of about 500 grams per mole, the preferred plasma concentration in molarity is from about 2 micromolar (μM) to about 5 millimolar (mM) and preferably about 100 μM to 1 mM antagonist. Stated differently, the dosage per body weight can vary from about 0.1 mg/kg to about 300 mg/kg, and preferably from about 0.2 mg/kg to about 200 mg/kg, in one or more dose administrations daily, for one or several days.

The polypeptides or peptidomimetics of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, polypeptides or peptidomimetics of the invention can be administered intravenously, intraperitoneally, intramuscularly, subcutaneously, intracavity, transdermally, and can be delivered by peristaltic means. The antagonist can also be administered orally.

The therapeutic compositions containing a polypeptide or peptidomimetic of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

In one preferred embodiment as shown in the Examples, the αᵥβ₃ antagonist is administered in a single dose intravenously.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgement of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

As demonstrated by the present Examples, inhibition of angiogenesis and tumor regression occurs as early as 7 days after the initial contacting with antagonist. Additional or prolonged exposure to antagonist is preferable for 7 days to 6 weeks, preferably about 14 to 28 days.

In a related embodiment, the Examples demonstrate the relationship between inhibition of αᵥβ₃ and induction of apoptosis in the neovasculature cells bearing αᵥβ₃. Thus, the invention also contemplates methods for inhibition of apoptosis in neovasculature of a tissue. The method is practiced substantially as described herein for inhibition of angiogenesis in all tissues and conditions described therefor. The only noticeable difference is one of timing of effect, which is that apoptosis is manifest quickly, typically about 48 hours after contacting antagonist, whereas inhibition of angiogenesis and tumor regression is manifest more slowly, as described herein. This difference affects the therapeutic regimen in terms of time of administration, and effect desired. Typically, administration for apoptosis of neovasculature can be for 24 hours to about 4 weeks, although 48 hours to 7 days is preferred.

D. Therapeutic Compositions

The present invention contemplates therapeutic compositions useful for practicing the therapeutic methods described herein. Therapeutic compositions of the present invention contain a physiologically tolerable carrier together with an αᵥβ₃ antagonist as described herein, dissolved or dispersed therein as an active ingredient. In a preferred embodiment, the therapeutic αᵥβ₃ antagonist composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric acid (HCl) or phosphoric acid, or such organic acids as acetic acid, tartaric acid, mandelic acid, trifluroacetic acid (TFA), and the like. Salts formed with the free carboxyl groups can also be derived from inorganic bases such as, for example, sodium hydroxide, potassium hydroxide, ammonium hydroxide (i.e., aqueous ammonia), calcium hydroxide, or ferric hydroxide, and such organic bases as isopropylamine, trimethylamine, 2-ethylamino ethanol, histidine, procaine, and the like.

Particularly preferred are the acid addition salts of TFA and HCl, when used in the preparation of cyclic polypeptide αᵥβ₃ antagonists. Representative salts of peptides are described in the Examples.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

A therapeutic composition contains an angiogenesis-inhibiting amount of an αᵥβ₃ antagonist of the present invention, typically formulated to contain an amount of at least 0.1 weight percent of antagonist per weight of total therapeutic composition. A weight percent is a ratio by weight of inhibitor to total composition. Thus, for example, 0.1 weight percent is 0.1 grams of inhibitor per 100 grams of total composition.

E. Antagonists of Integrin αᵥβ₃

αᵥβ₃ antagonists are used in the present methods for inhibiting angiogenesis in tissues, and can take a variety of forms that include compounds which interact with αᵥβ₃ in a manner such that functional interactions with natural αᵥβ₃ ligands are interfered. Exemplary antagonists include analogs of αᵥβ₃ derived from the ligand binding site on αᵥβ₃, mimetics of either αᵥβ₃ or a natural ligand of αᵥβ₃ that mimic the structural region involved in αᵥβ₃-ligand binding interactions, polypeptides having a sequence corresponding to a functional binding domain of the natural ligand specific for αᵥβ₃, particularly corresponding to the RGD-containing domain of a natural ligand of αᵥβ₃, and antibodies which immunoreact with either αᵥβ₃ or the natural ligand, all of which exhibit antagonist activity as defined herein.

E1. Polypeptides

In one embodiment, the invention contemplates αᵥβ₃ antagonists in the form of polypeptides. A polypeptide (peptide) αᵥβ₃ antagonist can have the sequence characteristics of either the natural ligand of αᵥβ₃ or αᵥβ₃ itself at the region involved in αᵥβ₃-ligand interaction and exhibits αᵥβ₃ antagonist activity as described herein. A preferred αᵥβ₃ antagonist peptide contains the RGD tripeptide and corresponds in sequence to the natural ligand in the RGD-containing region.

Preferred RGD-containing polypeptides have a sequence corresponding to the amino acid residue sequence of the RGD-containing region of a natural ligand of αᵥβ₃ such as fibrinogen, vitronectin, von Willebrand factor, laminin, thrombospondin, and the like ligands. The sequence of these αᵥβ₃ ligands are well known. Thus, an αᵥβ₃ antagonist peptide can be derived from any of the natural ligands, although fibrinogen and vitronectin are preferred.

A particularly preferred αᵥβ₃ antagonist peptide preferentially inhibits αᵥβ₃ binding to its natural ligand(s) when compared to other integrins, as described earlier. These αᵥβ₃-specific peptides are particularly preferred at least because the specificity for αᵥβ₃ reduces the incidence of undesirable side effects such as inhibition of other integrins. The identification of preferred αᵥβ₃ antagonist peptides having selectivity for αᵥβ₃ can readily be identified in a typical inhibition of binding assay, such as the ELISA assay described in the Examples.

A polypeptide of the present invention typically comprises no more than about 100 amino acid residues, preferably no more than about 60 residues, more preferably no more than about 30 residues. Peptides can be linear or cyclic, although particularly preferred peptides are cyclic.

Where the polypeptide is greater than about 100 residues, it is typically provided in the form of a fusion protein or protein fragment, as described herein.

Preferred cyclic and linear peptides and their designations are shown in Table 1 in the Examples.

It should be understood that a subject polypeptide need not be identical to the amino acid residue sequence of a αᵥβ₃ natural ligand, so long as it includes the required sequence and is able to function as an αᵥβ₃ antagonist in an assay such as those described herein.

A subject polypeptide includes any analog, fragment or chemical derivative of a polypeptide whose amino acid residue sequence is shown herein so long as the polypeptide is an αᵥβ₃ antagonist. Therefore, a present polypeptide can be subject to various changes, substitutions, insertions, and deletions where such changes provide for certain advantages in its use. In this regard, αᵥβ₃ antagonist polypeptide of this invention corresponds to, rather than is identical to, the sequence of a recited polypeptide where one or more changes are made and it retains the ability to function as an αᵥβ₃ antagonist in one or more of the assays as defined herein.

Thus, a polypeptide can be in any of a variety of forms of peptide derivatives, that include amides, conjugates with proteins, cyclic peptides, polymerized peptides, analogs, fragments, chemically modified peptides, and the like derivatives.

The term “analog” includes any polypeptide having an amino acid residue sequence substantially identical to a sequence specifically shown herein in which one or more residues have been conservatively substituted with a functionally similar residue and which displays the αᵥβ₃ antagonist activity as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The phrase “conservative substitution” also includes the use of a chemically derivatized residue in place of a non-derivatized residue provided that such polypeptide displays the requisite inhibition activity.

A “chemical derivative” refers to a subject polypeptide having one or more residues chemically derivatized by reaction of a functional side group. In additioin to side group derivitations, a chemical derivative can have one or more backbone modifications including α-amino substitutions such as N-methyl, N-ethyl, N-propyl and the like, and a-carbonyl substitutions such as thioester, thioamide, guanidino and the like. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides of the present invention also include any polypeptide having one or more additions and/or deletions or residues relative to the sequence of a polypeptide whose sequence is shown herein, so long as the requisite activity is maintained.

A particularly preferred derivative is a cyclic peptide according to the formula cyclo(Arg-Gly-Asp-D-Phe-NMeVal), SEQ ID NO: 15, abbreviated c(RGDf-NMeV), in which there is an N-methyl substituted a-amino group on the valine residue of the peptide and cyclization has joined the primary amino and carboxy termini of the peptide.

The term “fragment” refers to any subject polypeptide having an amino acid residue sequence shorter than that of a polypeptide whose amino acid residue sequence is shown herein.

When a polypeptide of the present invention has a sequence that is not identical to the sequence of an αᵥβ₃ natural ligand, it is typically because one or more conservative or non-conservative substitutions have been made, usually no more than about 30 number percent, and preferably no more than 10 number percent of the amino acid residues are substituted. Additional residues may also be added at either terminus of a polypeptide for the purpose of providing a “linker” by which the polypeptides of this invention can be conveniently affixed to a label or solid matrix, or carrier.

Labels, solid matrices and carriers that can be used with the polypeptides of this invention are described hereinbelow.

Amino acid residue linkers are usually at least one residue and can be 40 or more residues, more often 1 to 10 residues, but do not form αᵥβ₃ ligand epitopes. Typical amino acid residues used for linking are tyrosine, cysteine, lysine, glutamic and aspartic acid, or the like. In addition, a subject polypeptide can differ, unless otherwise specified, from the natural sequence of an αᵥβ₃ ligand by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

Any polypeptide of the present invention may be used in the form of a pharmaceutically acceptable salt. Suitable acids which are capable of forming salts with the peptides of the present invention include inorganic acids such as trifluoroacetic acid (TFA) hydrochloric acid (HCl), hydrobromic acid, perchloric acid, nitric acid, thiocyanic acid, sulfuric acid, methane sulfonic acid, acetic acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the polypeptides of the present invention include inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

In addition, a peptide of this invention can be prepared as described in the Examples without including a free ionic salt in which the charged acid or base groups present in the amino acid residue side groups (e.g., Arg, Asp, and the like) associate and neutralize each other to form an inner salt compound.

A polypeptide of the present invention also referred to herein as a subject polypeptide, can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”, W.H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis, each of which is incorporated herein by reference. Appropriate protective groups usable in such synthesis are described in the above texts and in J. F. W. McOmie, “Protective Groups in Organic Chemistry”, Plenum Press, New York, 1973, which is incorporated herein by reference.

In general, the solid-phase synthesis methods contemplated comprise the sequential addition of one or more amino acid residues or suitably protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group such as lysine.

Using a solid phase synthesis as exemplary, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above may be reacted to form their corresponding cyclic peptides. An exemplary method for preparing a cyclic peptide is described by Zimmer et al., Peptides 1992, pp. 393-394, ESCOM Science Publishers, B.V., 1993. Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tert-butoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

Alternative methods for cyclic peptide synthesis are described by Gurrath et al., Eur. J. Biochem., 210:911-921 (1992), and described in the Examples.

In addition, the αᵥβ₃ antagonist can be provided in the form of a fusion protein. Fusion proteins are proteins produced by recombinant DNA methods as described herein in which the subject polypeptide is expressed as a fusion with a second carrier protein such as a glutathione sulfhydryl transferase (GST) or other wll known carrier. Preferred fusion proteins comprise an MMP-2 polypeptide described herein. The preparation of a MMP-2 fusion protein is described in the Examples.

Particularly preferred peptides and derivative peptides for use in the present methods are c-(GrGDFV) (SEQ ID NO: 4), c-(RGDfV) (SEQ ID NO: 5), c-(RADfV) (SEQ ID NO: 6), c-(RGDFv) (SEQ ID NO: 7), c-(RGDf-NMeV)(SEQ ID NO: 15) and linear peptide YTAECKPQVTRGDVF (SEQ ID NO: 8), where “c-” indicates a cyclic peptide, the upper case letters are single letter code for an L-amino acid and the lower case letters are single letter code for D-amino acid. The amino acid residues sequence of these peptides are also shown in SEQ ID NO:s 4, 5, 6, 7, 15 and 8, respectively.

Also preferred are polypeptides derived from MMP-2 described herein, having sequences shown in SEQ ID NO:s 17-28 and 45.

E2. αᵥβ₃-Specific Peptidomimetic Compounds

The present invention demonstrates that αᵥβ₃ antagonists generally can be used in the present invention, which antagonists can include polypeptides, antibodies and other molecules, designated “mimetics” or “peptidomimetics”, which have the capacity to interefere with αᵥβ₃ function. Particularly preferred are antagonists which specifically interfere with αᵥβ₃ function, and do not interfere with function of other integrins.

In this context it is appreciated that a variety of reagents may be suitable for use in the present methods, so long as these reagents posses the requisite biological activity. These reagents are generically referred to a mimetics because they possess the ability to “mimic” a peptide binding domain on either αᵥβ₃ or the αᵥβ₃ ligand involved in the functional interaction of the receptor and ligand, and thereby interfere with (i.e., inhibit) normal function.

An αᵥβ₃ mimetic is any molecule, other than an antibody or ligand-derived peptide, which exhibits the above-described properties. It can be a synthetic peptide, an analog or derivative of a peptide, a compound which is shaped like the binding pocket of the above-described binding domain such as an organic mimetic molecule, or other molecule.

The design of an αᵥβ₃ mimetic can be conducted by any of a variety of structural analysis methods for drug-design known in the art, including molecular modelling, two-dimensional nuclear magnetic resonance (2-D NMR) analysis, x-ray crystallography, random screening of peptide, peptide analog or other chemical polymer or compound libraries, and the like drug design methodologies.

In view of the broad structural evidence presented in the present specification which shows that an αᵥβ₃ antagonist can be a fusion polypeptide (e.g., an MMP-2 fusion protein), a small polypeptide, a cyclic peptide, a derivative peptide, an organic peptidomimetic molecule, or a monoclonal antibody, that are diversely different chemical structures which share the functional property of selective inhibition of αᵥβ₃, the structure of a subject αᵥβ₃ antagonist useful in the present methods need not be so limited, but includes any organic αᵥβ₃ antagonist, as defined herein.

Preferably the organic αᵥβ₃ antagonist is an organic peptidomimetic compound having a basic group and an acidic group spaced from one another by a distance in the range of about 10 Angstroms to about 100 Angstroms.

Preferred organic peptidomimetic αᵥβ₃ antagonist compounds having a basic group and an acidic group spaced from one another by a distance in the range of about 10 Angstroms to about 100 Angstroms that are useful in the methods of the present invention have the following general formula (I):

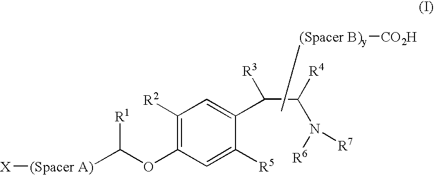

wherein

R¹ and R² are both H or together form a radical selected from the group consisting of —CH₂—C(O)—, ═C—C(O)—, and —C(O)—NH—;

R³ and R⁴ are both H or together form a covalent bond;

R⁵ and R⁶ are both H or, when R³ and R⁴ together form a covalent bond, R⁵ and R⁶ together form a covalent bond;

R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;

X is selected from the group consisting of 2-imidazolyl, 2-benzimidazolyl, N-guanidyl, N—(C₁-C₂) alkyl-substituted guanidyl, 2-pyridyl, 4-carbonimidophenyl, and 6-(2-methylaminopyridyl);

Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O)CH₂—, and —NHC(O)CH₂CH₂—;

Spacer B is a radical selected from the group consisting of —CH₂—, —CH₂CH₂—, and —CH(R⁸)CH₂—;

Z¹ and Z² are both covalent bonds to a bridging carbonyl group forming a cyclic urethane;

R⁸ is phenyl or 5-benzo-2, 1, 3-thiadiazolyl; and

Spacer B is covalently bonded to either of the carbon atoms bearing substituents R³ and R⁴; y is 0 or 1, with the proviso that when R⁵ and R⁶ form a covalent bond, y is 1.

Many of the compounds represented by formula (I) include chiral centers and can exist in optically active, isomeric forms. All enantiomers and diastereomers of compounds of formula (I) are useful in the methods of the present invention.

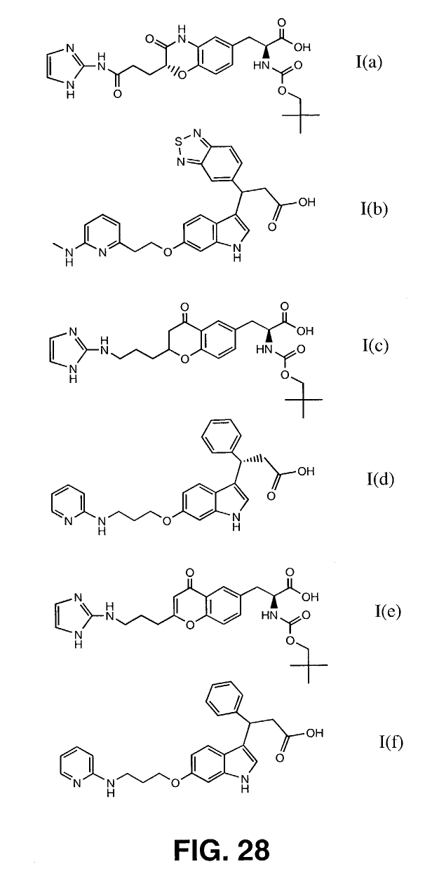

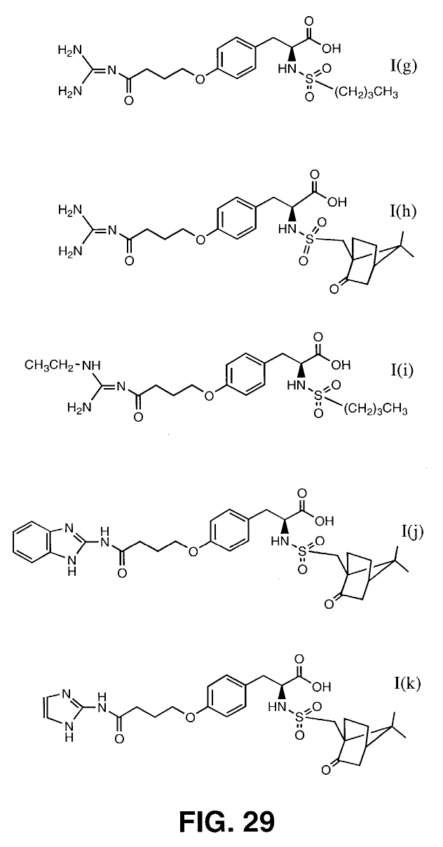

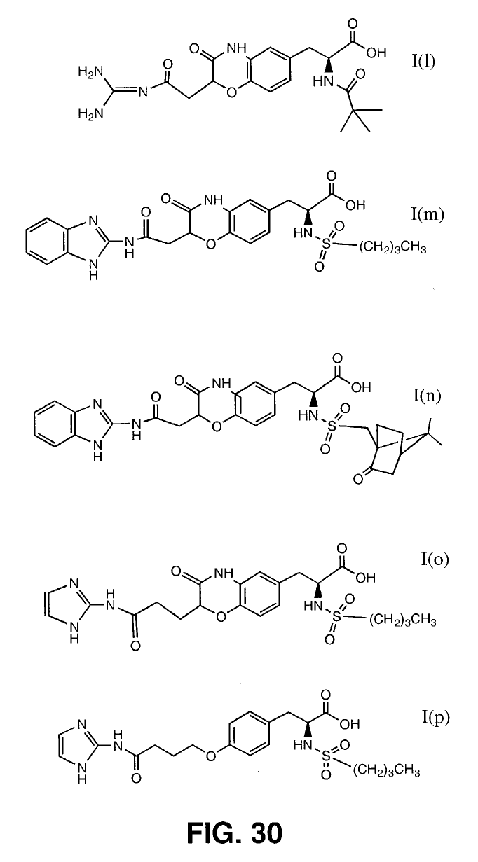

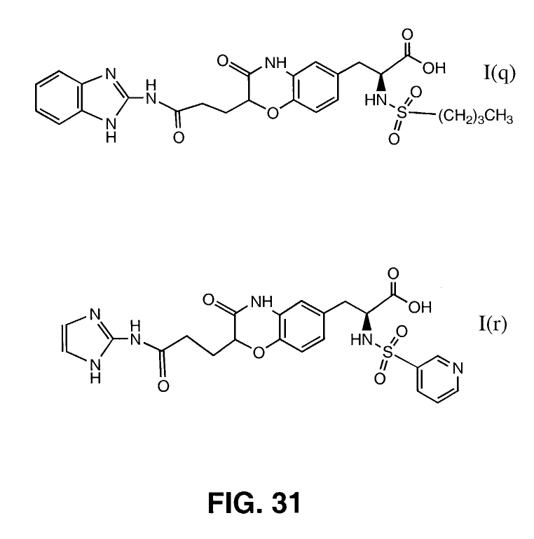

Particularly preferred examples of compounds of formula (I) that are useful in the methods of the present invention are illustrated in FIGS. 28-31 (e.g., Compounds I(a) through I(r)), as well Compounds 7, 9, 10, 12, 14, 16, 17 and 18 as described in Example 1.

Compounds of general formula (I) can be prepared by methods well known in the art. In particular, Compounds of formula (I), including Compounds I(a) through I(r) can be synthesized by the methods disclosed in U.S. Patent Publication No. 2001/0021709A1 to Diefenbach et al., U.S. Pat. No. 6,204,280 to Gante et al., Canadian Patent Application No. 2,241,149 to Diefenbach et al. and PCT Publication No. WO 01/58893 to Goodman et al.; the relevant disclosures of each of the foregoing being incorporated herein by reference.

F. Examples.

The following examples relating to this invention are illustrative and should not, of course, be construed as specifically limiting the invention. Moreover, such variations of the invention, now known or later developed, which would be within the purview of one skilled in the art are to be considered to fall within the scope of the present invention hereinafter claimed.

### EXAMPLE 1

Preparation of Synthetic Peptides.

### Ex. 1A. Synthesis Procedure

The linear and cyclic polypeptides listed in Table 1 were synthesized using standard solid-phase synthesis techniques as, for example, described by Merrifield, Adv. Enzymol., 32:221-96, (1969), and Fields, G. B. and Noble, R. L., Int. J. Peptide Protein Res., 35:161-214, (1990).

Two grams (g) of BOC-Gly-D-Arg-Gly-Asp-Phe-Val-OMe (SEQ ID NO: 1) were first dissolved in 60 milliliters (ml) of methanol to which was added 1.5 ml of 2 N sodium hydroxide solution to form an admixture. The admixture was then stirred for 3 hours at 20° C. After evaporation, the residue was taken up in water, acidified to pH 3 with diluted HCl and extracted with ethyl acetate. The extract was dried over Na₂SO₄, evaporated again and the resultant BOC-Gly-D-Arg-Gly-Asp-Phe-Val (SEQ ID NO: 2) was stirred at 20° C. for 2 hours with 20 ml of 2 N HCl in dioxane. The resultant admixture was evaporated to obtain Gly-D-Arg-Gly-Asp-Phe-Val (SEQ ID NO: 3) that was subsequently dissolved in a mixture of 1800 ml of dichloromethane and 200 ml of dimethylformamide (DMF) followed by cooling to 0° C. Thereafter, 0.5 9 of dicyclohexylcarbodiimide (DCCI), 0.3 g of 1-hydroxybenzotriazole (HOBt) and 0.23 ml of N-methylmorpholine were added successively with stirring.

The resultant admixture was stirred for a further 24 hours at 0° C. and then at 20° C. for another 48 hours. The solution was concentrated and treated with a mixed bed ion exchanger to free it from salts. After the resulting resin was removed by filtration, the clarified solution was evaporated and the residue was purified by chromatography resulting in the recovery of cyclo(-Gly-D-Arg-Gly-Asp-Phe-Val) (SEQ ID NO: 4).

The following peptides, listed in Table 1 using single letter code amino acid residue abbreviations and identified by a peptide number designation, were obtained analogously: cyclo(Arg-Gly-Asp-D-Phe-Val) (SEQ ID NO: 5); cyclo(Arg-Ala-Asp-D-Phe-Val) (SEQ ID NO: 6); cyclo(Arg-D-Ala-Asp-Phe-Val) (SEQ ID NO: 9); cyclo(Arg-Gly-Asp-Phe-D-Val) (SEQ ID NO: 7); and cyclo (Arg-Gly-Asp-D-Phe-NMeVal) (methylation is at the alpha-amino nitrogen of the amide bond of the valine residue) (SEQ ID NO: 15).

A peptide designated as 66203, having an identical sequence to that of peptide 62184, only differed from the latter by containing the salt HCl rather than the TFA salt present in 62184. The same is true for the peptides 69601 and 62185 and for 85189 and 121974.

### Ex. 1B. Alternate Synthesis Procedure

### Ex. 1B(1) Synthesis of Cyclo-(Arg-Gly-Asp-DPhe-NmeVal) (SEQ ID NO: 15), TFA Salt

Nα-Fmoc-Arg(NG-Mtr)-Gly-Asp(OBut)-DPhe-NMeVal sodium salt (SEQ ID NO: 46) is synthesized using solid-phase Merrifield-type procedures by sequentially adding NMeVal, DPhe, Asp(OBut), Gly and Fmoc-Arg(Mtr) in a step-wise manner to a 4-hydroxymethyl-phenoxymethyl-polystyrene resin (Wang type resin) (customary Merrifield-type methods of peptide synthesis are applied as described in Houben-Weyl, 1.c., Volume 15/II, Pages 1 to 806 (1974). The polystyrene resin and amino acid residues precursors are commercially available from Aldrich, Sigma or Fluka chemical companies). After completion of sequential addition of the amino acid residues, the resin is then eliminated from the peptide chain using a 1:1 mixture of TFA/dichloromethane which provides the Nα-Fmoc-Arg(N^{G}-Mtr)-Gly-Asp(OBut)-DPhe-NMeVal product (SEQ ID NO: 46). The Fmoc group is then removed with a 1:1 mixture of piperidine/DMF which provides the crude Arg(N GMtr)-Gly-Asp(OBut)-DPhe-NMeVal (SEQ ID NO: 47) precursor which is then purified by HPLC in the customary manner.

For cyclization, a solution of 0.6 g of Arg(N^{G}-Mtr)-Gly-Asp(OBut)-DPhe-NMeVal (SEQ ID NO: 47, synthesized above) in 15 ml of DMF (dimethylformamide; Aldrich) is diluted with 85 ml of dichloromethane (Aldrich), and 50 mg of NaHCO₃ are added. After cooling in a dry ice/acetone mixture, 40 μl of diphenylphosphoryl azide (Aldrich) are added. After standing at room temperature for 16 hours, the solution is concentrated. The concentrate is gel-filtered (Sephadex G10 column in isopropanol/water 8:2) and then purified by HPLC in the customary manner. Treatment with TFA (trifluoroacetic acid)/H₂O (98:2) gives cyclo-(Arg-Gly-Asp-DPhe-NmeVal)×TFA (SEQ ID NO: 15, TFA salt), which is then purified by HPLC in the customary manner; RT=19.5; FAB-MS (M+H): 589.

### Ex. 1B(2) Synthesis of “Inner Salt”

TFA salt is removed from the above-produced cyclic peptide by suspending the cyclo-(Arg-Gly-Asp-DPhe-NmeVal)×TFA SEQ ID NO: 15, TFA salt) in water followed by evaporation under vacuum to remove the TFA. The cyclic peptide formed is referred to as an inner salt and is designated cyclo-(Arg-Gly-Asp-DPhe-NMeVal), SEQ ID NO: 15. The term inner salt is used because the cyclic peptide contains two oppositely charged residues which intra-electronically counterbalance each other to form an overall noncharged molecule. One of the charged residues contains an acid moiety and the other charged residue contains an amino moiety. When the acid moiety and the amino moiety are in close proximity to one another, the acid moiety can be deprotonated by the amino moiety which forms a carboxylate/ammonium salt species with an overall neutral charge.

### Ex. 1B(3) HCl Treatment to Give Cyclo-(Arg-Gly-Asp-DPhe-NMeVal)×HCl (SEQ ID NO: 15, HCl Salt)

80 mg of cyclo-(Arg-Gly-Asp-DPhe-NMeVal) (SEQ ID NO: 15) are dissolved in 0.01 M HCl five to six times and freeze dried after each dissolving operation. Subsequent purification by HPLC affords the HCl salt; FAB-MS (M+H): 589.

### Ex. 1B(4) Methane Sulfonic Acid Treatment to Give Cyclo-(Arg-Gly-Asp-DPhe-NMeVal)×MeSO₃H (SEQ ID NO: 15, Methanesulfonate Salt)

80 mg of cyclo-(Arg-Gly-Asp-DPhe-NMeVal) (SEQ ID NO: 15) are dissolved in 0.01 M MeSO₃H (methane sulfonic acid) five to six times and freeze dried after each dissolving operation. Subsequent purification by HPLC affords the methanesulfonate salt; RT=17.8 ; FAB-MS (M+H): 589.

Alternative methods of cyclization include derivatizing the side group chains of an acyclic peptide precursor with sulfhydryl moieties, and when exposed to slightly higher than normal physiological pH conditions (pH 7.5), intramolecularly forms disulfide bonds with other sulfhydryl groups present in the molecule to form a cyclic peptide. Additionally, the C-terminus carboxylate moiety of an acyclic peptide precurosor can be reacted with a free sulfhydryl moiety present within the molecule for producing thioester cyclized peptides.

In inhibition of angiogenesis assays as described in Example 5 where the synthetic peptides were used, the 66203 peptide in HCl was slightly more effective in inhibiting angiogenesis than the identical peptide in TFA.

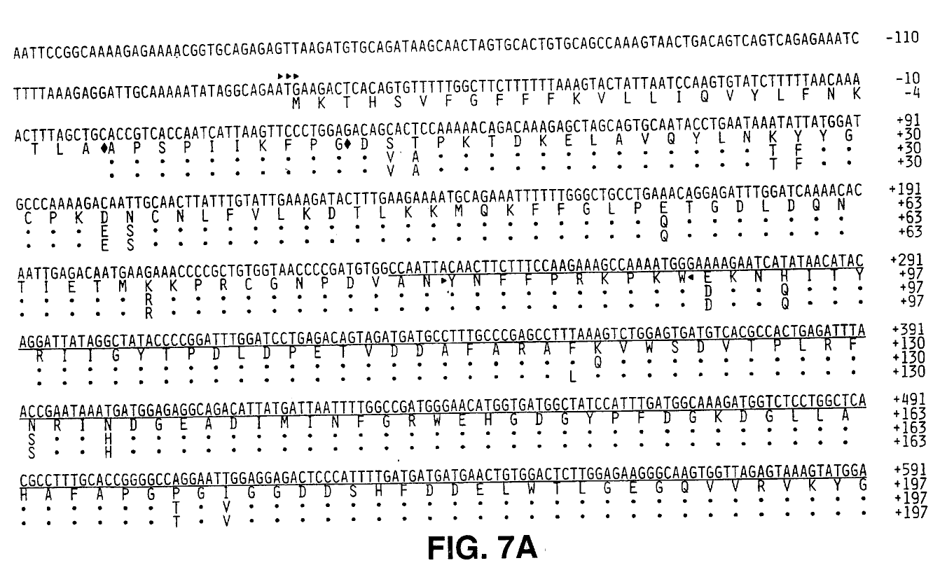

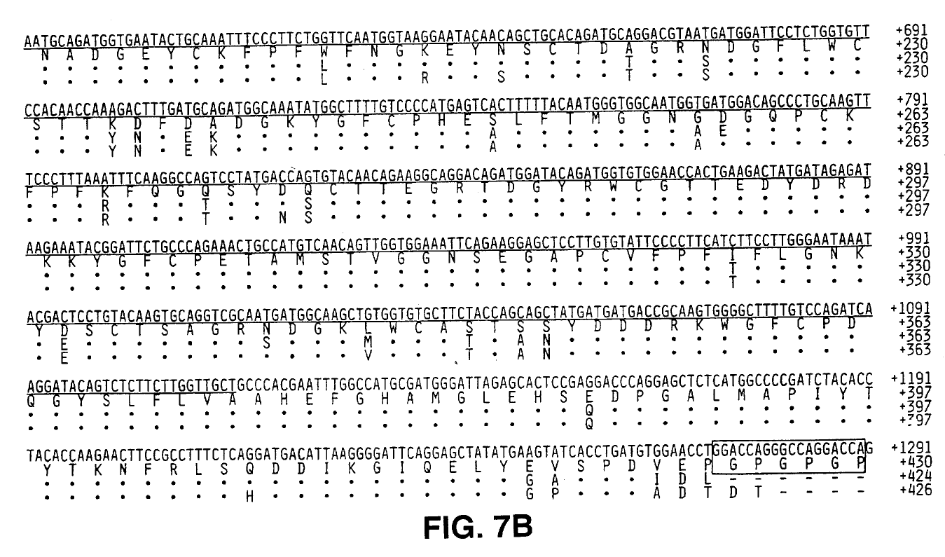

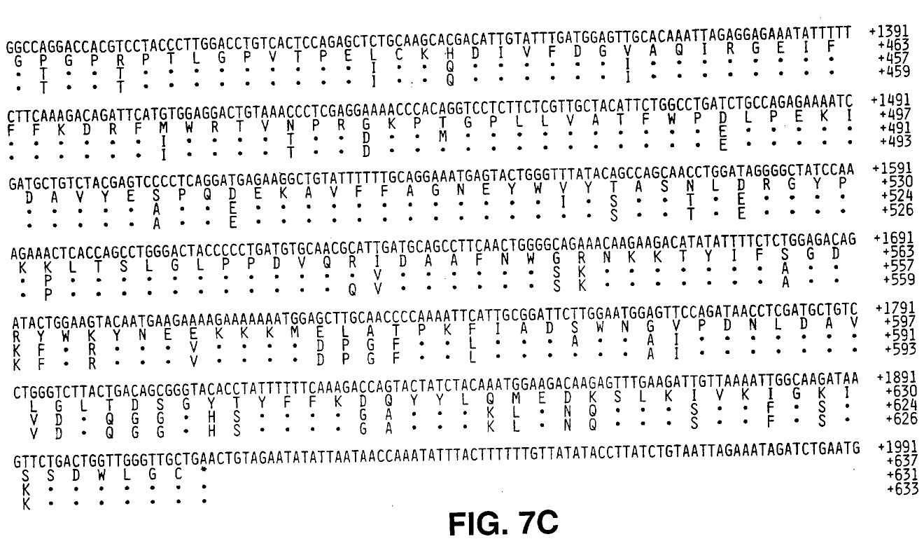

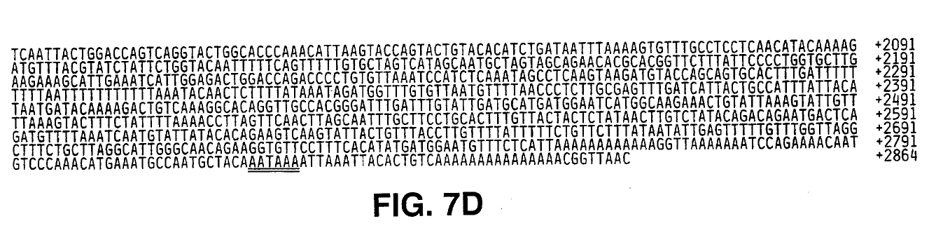

TABLE 1

| Peptide | | | SEQ ID |
|---|---|---|---|
| Designation | | Amino Acid Sequence | NO: |
| 62181 | | cyclo (GrGDFV) | 4 |
| 62184 | (66203*) | cyclo (RGDfV) | 5 |
| 62185 | (69601*) | cyclo (RADfV) | 6 |
| 62187 | | cyclo (RGDFv) | 7 |
| 62880 | | YTAECKPQVTRGDVF | 8 |
| 62186 | | cyclo (RaDFV) | 9 |
| 62175 | | cyclo (ARGDfL) | 10 |
| 62179 | | cyclo (GRGDfL) | 11 |
| 62411 | | TRQVVCDLGNPM | 12 |
| 62503 | | GVVRNNEALARLS | 13 |
| 62502 | | TDVNGDGRHDL | 14 |
| 121974 | (85189*) | cyclo (RDGf-NH₂Me-V) | 15 |
| 112784 | | cyclo (RGEf-NH₂Me-V) | 16 |
| huMMP-2 | (410-631)** | | 17 |
| huMMP-2 | (439-631)** | | 18 |
| huMMP-2 | (439-512)** | | 19 |
| huMMP-2 | (439-546)** | | 20 |
| huMMP-2 | (510-631)** | | 21 |
| huMMP-2 | (543-631)** | | 22 |
| chMMP-2 | (410-637)*** | | 23 |
| chMMP-2 | (445-637)*** | | 24 |
| chMMP-2 | (445-518)*** | | 25 |
| chMMP-2 | (445-552)*** | | 26 |
| chMMP-2 | (516-637)*** | | 27 |
| chMMP-2 | (549-637)*** | | 28 |
| *The peptides designated with an asterisk are prepared in HCl and are identical in sequence to the peptide designated on the same line; the peptides without an asterisk are prepared in TFA. Lower case letters indicate a D-amino acid; capital letters indicate a L-amino acid. | | | |
| **The human MMP-2 amino acid residue sequences for synthetic peptides are indicated by the corresponding residue positions shown in FIGS. 7A through 7D. (MMP-2 refers to a member of the family of matrix metalloproteinase enzymes). The human MMP-2 sequences are listed with the natural cysteine residues but are not listed with engineered cysteine residues as described for the fusion peptides. The non-natural cysteine residues were substituted for the natural amino acid residue at the | | | |
| #indicated residue positions in order to facilitate solubility of the synthetic as well as expressed fusion proteins and to ensure proper folding for presentation of the binding site. | | | |

***The chicken MMP-2 amino acid residue sequences for synthetic peptides are indicated by the corresponding residue positions shown in FIGS. 7A through 7D. The chicken MMP-2 sequences are listed with the natural cysteine residues but not with the engineered cysteine residues as described for the fusion peptides as described above.

### EXAMPLE 2

Identification of αᵥβ₃-Specific Synthetic Peptides Detected by Inhibition of Cell Attachment and by a Ligand-Receptor Binding Assay.

### Ex. 2A Inhibition of Cell Attachment

As one means to determine integrin receptor specificity of the antagonists of this invention, inhibition of cell attachment assays were performed as described below.

Briefly, CS-1 hamster melanoma cells lacking expression of αᵥβ₃ and αᵥβ₅ were first transfected with an plasmid for expressing the 3 subunit as previously described by Filardo et al., J. Cell Biol., 130:441-450 (1995). Specificity of potential αᵥβ₃ antagonists was determined by the ability to block the binding of αᵥβ₃-expressing CS-1 cells to VN or laminin coated plates. As an example of a typical assay, the wells were first coated with 10 μg/ml substrate overnight. After rinsing and blocking with 1% heat-denatured BSA in PBS at room temperature for 30 minutes, peptide 85189 (SEQ ID NO: 15) over a concentration range of 0.0001 μM to 100 μM, was separately mixed with CS-1 cells for applying to wells with a cell number of 50, 000 cells/well. After a 10-15 minute incubation at 37° C., the solution containing the cells and peptides was discarded. The number of attached cells was then determined following staining with 1% crystal violet. Cell associated crystal violet was eluted by the addition of 100 microliters (μl) of 10% acetic acid. Cell adhesion was quantified by measuring the optical density of the eluted crystal violet at a wave length of 600 nm.

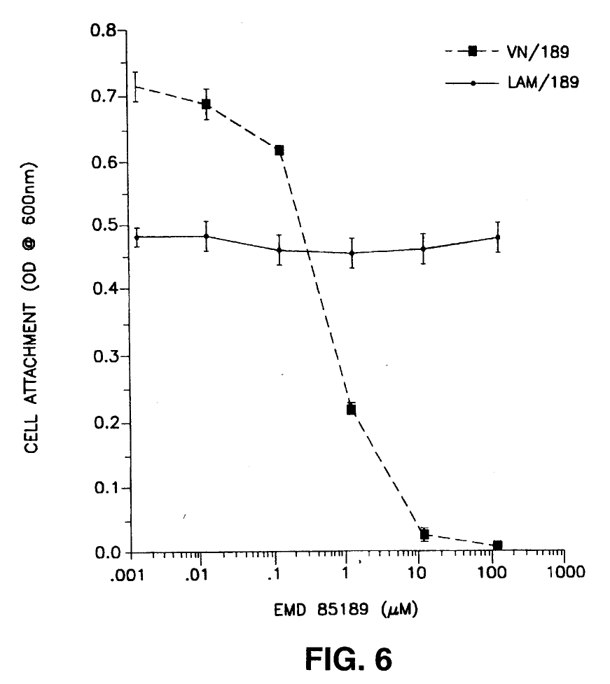

FIG. 6 shows the result of a typical assay with an αᵥβ₃ antagonist, here peptide 85189. No inhibition was detected with the peptide on laminin-coated surfaces. In contrast, complete inhibition of binding was obtained on VN-coated surfaces with a peptide concentration of 10 μM or greater, as shown with the dose-response curve.

Similar assays were performed with fusion proteins containing various regions of the MMP-2 protein. The MMP-2-derived polypeptides include regions of the C-terminus of MMP-2 active in the binding interaction with αᵥβ₃ and thereby capable of inhibiting MMP-2 activation and associated activities. These polypeptides are prepared either as synthetic polypeptides having a sequence derived from the C-terminal domain of MMP-2 as described in Example 1 or as fusion proteins including all or a portion of the C-terminal domain of MMP-2, prepared as described below. MMP-2 C-terminal molecules are presented for both chicken and human specific sequences.

The chicken-derived MMP-2 C-terminal domain, also referred to as the hemopexin domain immediately contiguous with the hinge region, comprises the amino acid residues 445-637 of MMP-2. The complete nucleotide and encoded amino acid sequence of chicken MMP-2 is described below. The human MMP-2 nucleotide and encoded amino acid sequence is also described below. The C-terminal domain in the human MMP-2 that corresponds to the chicken region of 445-637 begin at amino acid residue 439 and ends with 631 due to six missing residues from the human sequence as shown in FIGS. 7A-7D. Both human- and chicken-derived C-terminal MMP-2 synthetic peptides for use in practicing the methods of this invention are listed in Table 1. The amino acid residue sequences of the synthetic peptides are the same as those generated by the recombinant fusion protein counterparts but without the GST fusion component. The C-terminal MMP-2 fusion proteins derived from both chicken and human are prepared as described below.

A MMP-2 fusion protein is a chimeric polypeptide having a sequence of MMP-2 C-terminal domain or a portion thereof fused (operatively linked by covalent peptide bond) to a carrier (fusion) protein, such as glutathione sulfhydryl transferase (GST).

To amplify various regions of chicken and human MMP-2, primer sequences were designed based on the known respective cDNA sequences of chicken and human MMP-2. The complete top strand of the cDNA nucleotide sequence of unprocessed chicken MMP-2, also referred to as progelatinase, is shown in FIGS. 7A-7D along with the deduced amino acid sequence shown on the second line (Aimes et al., Biochem. J., 300:729-736, 1994). The third and fourth lines of the FIG. respectively show the deduced amino acid sequence of human (Collier et al., J. Biol. Chem., 263:6579-6587 (1988)) and mouse MMP-2 (Reponen et al., J. Biol. Chem., 267:7856-7862 (1992)). Identical residues are indicated by dots while the differing residues are given by their one letter IUPAC lettering. Missing residues are indicated by a dash. The numbering of the amino acid residues starts from the first residue of the proenzyme, with the residues of the signal peptide being given negative numbers. The nucleotide sequence is numbered accordingly. The putative initation of translation (ATG) is marked with three forward arrowheads and the translation termination signal (TGA) is indicated by an asterisk. The amino terminal sequences for the chicken proenzyme and active enzyme are contained with diamonds and single arrowheads. The chicken progelatinase nucleotide and amino acid residue sequences are listed together as SEQ ID NO: 29 while the encoded amino acid residue sequence is listed separately as SEQ ID NO: 30.

Templates for generating amplified regions of chicken MMP-2 were either a cDNA encoding the full-length mature chicken MMP-2 polypeptide provided by Dr. J. P. Quigley of the State University of New York at Stoney Brook, N.Y. or a cDNA generated from a total cellular RNA template derived by standard techniques from an excised sample of chicken chorioallantoic membrane tissue. For the latter, the cDNA was obtained with MuLV reverse transcriptase and a downstream primer specific for the 3′-terminal nucleotides, 5′ATTGAATTCTTCTACAGTTCA3′ (SEQ ID NO: 31), the 5′ and 3′ ends of which was respectively complementary to nucleotides 1932-1912 of the published chick MMP-2 sequence. Reverse transcriptase polymerase chain reaction (RT-PCR) was performed according to the specifications of the manufacturer for the GeneAmp RNA PCR Kit (Perkin Elmer). The primer was also engineered to contain an internal EcoRI restriction site.

From either of the above-described cDNA templates, a number of C-terminal regions of chicken MMP-2, each having the natural cysteine residue at position 637 at the carboxy terminus, were obtained by PCR with the 3′ primer listed above (SEQ ID NO: 31) paired with one of a number of 5′ primers listed below. The amplified regions encoded the following MMP-2 fusion proteins, having sequences corresponding to the amino acid residue positions as shown in FIGS. 7A-7D and also listed in SEQ ID NO: 30: 1) 203-637; 2) 274-637; 3) 292-637; 4) 410-637; 5) 445-637. Upstream or 5′ primers for amplifying each of the nucleotide regions for encoding the above-listed MMP-2 fusion proteins were designed to encode the polypeptide start sites 3′ to an engineered, i.e., PCR-introduced, internal BamHI restriction site to allow for directional ligation into either pGEX-1λT or pGEX-3× expression vectors. The 5′ primers included the following sequences, the 5′ and 3′ ends of which correspond to the indicated 5′ and 3′ nucleotide positions of the chicken MMP-2 sequence as shown in FIG. 7A-7D (the amino acid residue position start sites are also indicated for each primer): 1) Nucleotides 599-619, encoding a 203 start site 5′ATGGGATCCACTGCAAATTTC3′ (SEQ ID NO: 32); 2) Nucleotides 809-830, encoding a 274 start site 5′GCCGGATCCATGACCAGTGTA3′ (SEQ ID NO: 33); 3) Nucleotides 863-883, encoding a 292 start site 5′GTGGGATCCCTGAAGACTATG3′ (SEQ ID NO: 34); 4) Nucleotides 1217-1237, encoding a 410 start 5′AGGGGATCCTTAAGGGGATTC3′ (SEQ ID NO: 35); and 5) Nucleotides 1325-1345, encoding a 445 start site 5′CTCGGATCCTCTGCAAGCACG3′ (SEQ ID NO: 36).

The indicated nucleotide regions of the template cDNA were subsequently amplified for 35 cycles (annealing temperature 55° C.) according to the manufacturer's instructions for the Expand High Fidelity PCR System (Boehringer Mannheim). The resulting PCR products were gel-purified, digested with BamHI and EcoRI restriction enzymes, and repurified before ligation into either pGEX-1λT or pGEX-3× vector (Pharmacia Biotech, Uppsala, Sweden) which had been similarly digested as well as dephosphorylated prior to the ligation reaction. The choice of plasmid was based upon the required reading frame of the amplification product. Competent E. coli strain BSJ72 or BL21 cells were transformed with the separate constructs by heat shock. The resulting colonies were screened for incorporation of the respective MMP-2 fusion protein-encoding plasmid by PCR prior to dideoxy sequencing of positive clones to verify the integrity of the introduced coding sequence. In addition, verification of incorporation of plasmid was confirmed by expression of the appropriately-sized GST-MMP-2 fusion protein.

Purification of each of the recombinant GST-MMP-2 fusion proteins was performed using IPTG-induced log-phase cultures essentially as described by the manufacturer for the GST Gene Fusion System (Pharmacia Biotech). Briefly, recovered bacteria were lysed by sonication and incubated with detergent prior to clarification and immobilization of the recombinant protein on sepharose 4B-coupled glutathione (Pharmacia Biotech). After extensive washing, the immobilized fusion proteins were separately eluted from the affinity matrix with 10 mM reduced glutathione in 50 mM Tris-HCl, pH 8.0, and dialyzed extensively against PBS to remove residual glutathione prior to use.

Prior attempts to produce fusion proteins between chicken MMP-2 residues 445 and 637 that only had one encoded cysteine residue resulted in insoluble products. Therefore, in order to generate additional soluble MMP-2 fusion proteins derived from the C-terminal region that did not include an endogenous terminal cysteine residue as present in the previously-described fusion protein, nucleotide sequences were introduced into amplified MMP-2 regions to encode a cysteine residue if necessary depending on the particular fusion protein. A cysteine residue is naturally present in the chicken MMP-2 sequence at position 446 and at position 637. In the human sequence, these positions correspond respectively to 440 and 631. Therefore, fusion proteins were designed to contain engineered terminal cysteine residues at the amino- or carboxy-terminus of the chicken MMP-2 sequences of interest so as to provide for disulfide-bonding with the naturally occurring cysteine at the other terminus, as required by the construct.

Oligonucleotide primers were accordingly designed to allow for amplification of chicken MMP-2 C-terminal regions for expression of soluble MMP-2/GST fusion proteins. Amplified chicken MMP-2 C-terminal regions included those for encoding amino acid residue positions 445-518, 445-552, 516-637 and 549-637. For fusion proteins containing residue 517, the naturally encoded tyrosine residue was substituted for a cysteine to allow for disulfide bonding with either cysteine at residue position 446 or 637. For fusion proteins containing residue 551, the naturally encoded tryptophan residue was substituted for a cysteine to allow for disulfide bonding with either naturally encoded cysteine at residue position 446 or 637.

Briefly, the pGEX-3× plasmid construct encoding the recombinant GST/MMP-2(410-637) fusion protein prepared above was used as a template for amplification according to the manufacturer's protocol for the Expand High Fidelity PCR Kit (Boehringer Mannheim) utilizing a set of oligonucleotide primers whose design was based on the published chicken MMP-2 sequence (also shown in FIGS. 11A and 11B. One upstream primer, designed to encode a chicken MMP-2 protein start site at position 445 after an engineered internal BamHI endonuclease restriction site for insertion into the pGEX-3× GST vector, had the nucleotide sequence (5′CTCGGATCCTCTGCAAGCACG3′ (SEQ ID NO: 37)). The 5′ and 3′ ends of the primer respectively corresponded to positions 1325-1345 of the chicken MMP-2 sequence in the FIGS. 7A-7D. Another upstream primer, designed to encode a chicken MMP-2 protein start site at position 516 after an engineered internal BamHI restriction site for insertion into the pGEX-1λT GST vector and to encode a cysteine residue at position 517, had the nucleotide sequence (5′GCAGGATCCGAGTGCTGGGTTTATAC3′ (SEQ ID NO: 38)). The 5′ and 3′ ends of the primer respectively corresponded to positions 1537-1562 of the chicken MMP-2 sequence. A third upstream primer, designed to encode a chicken MMP-2 protein start site at position 549 following an engineered internal EcoRI endonuclease restriction site for insertion into the pGEX-1λT GST vector and to encode a cysteine residue at position 551, had the nucleotide sequence (5′GCAGAATTCAACTGTGGCAGAAACAAG3′ (SEQ ID NO: 39)). The 5′ and 3′ ends of the primer respectively corresponded to positions 1639-1665 of the chicken MMP-2 sequence.

These upstream primers were separately used with one of the following downstream primers listed below to produce the above-described regions from the C-terminal domain of chicken MMP-2. A first downstream primer (antisense), designed to encode a chicken MMP-2 protein termination site at position 518, to encode a cysteine residue at position 517, and to contain an internal EcoRI endonuclease restriction site for insertion into a GST vector, had the nucleotide sequence (5′GTAGAATTCCAGCACTCATTTCCTGC3′ (SEQ ID NO: 40)). The 5′ and 3′ ends of the primer, written in the 5′-3′ direction, were respectively complementary in part to positions 1562-1537 of the chicken MMP-2 sequence. A second downstream primer, designed to encode a chicken MMP-2 protein termination site at position 552, to encode a cysteine residue at position 551, and to contain an internal EcoRI endonuclease restriction site for insertion into a GST vector, had the nucleotide sequence (5′TCTGAATTCTGCCACAGTTGAAGG3′ (SEQ ID NO: 41)). The 5′ and 3′ ends of the primer, written in the 5′-3′ direction, were respectively complementary in part to positions 1666-1643 of the chicken MMP-2 sequence. A third downstream primer, designed to encode a chicken MMP-2 protein termination site at position 637 and to contain an internal EcoRI endonuclease restriction site for insertion into a GST vector, had the nucleotide sequence (5′ATTGAATTCTTCTACAGTTCA3′ (SEQ ID NO: 42)). The 5′ and 3′ ends of the primer, written in the 5′-3′ direction, were respectively complementary in part to positions 1932-1912 of the chicken MMP-2 sequence.

The regions of the chicken MMP-2 carboxy terminus bounded by the above upstream and downstream primers, used in particular combinations to produce the fusion proteins containing at least one engineered cysteine residue as described above, were separately amplified for 30 cycles with an annealing temperature of 55° C. according to the manufacturer's instructions for the Expand High Fidelity PCR System (Boehringer Mannheim). The resulting amplification products were separately purified, digested with BamHI and or EcoRI restriction enzymes as necessary, and repurified before ligation into the appropriate GST fusion protein vector, either pGEX-3× or pGEX-1λT, as indicated above by the reading frame of the upstream oligonucleotide primer. For ligating the amplified MMP-2 products, the vectors were similarly digested as well as dephosphorylated prior to the ligation reaction. Competent E. coli strain BL21 cells were then separately transformed with the resultant MMP-2-containing vector constructs by heat shock. Resulting colonies were then screened for incorporation of the appropriate fusion protein-encoding plasmid by PCR and production of the appropriate sized GST-fusion protein prior to dideoxy sequencing of positive clones to verify the integrity of the introduced coding sequence. Purification of recombinant GST fusion proteins were then performed using IPTG-induced log-phase cultures essentially as described above for producing the other GST-MMP-2 fusion proteins.

The results of inhibition of cell attachment assays with various chicken MMP-2 proteins as well as with other peptides indicate that intact MMP-2, the fusion protein CTMMP-2(2-4) from residues 445-637 and peptide 66203 (SEQ ID NO: 5) but not MMP-2 (1-445) and control peptide 69601 inhibited β₃-expressing CS-1 cell adhesion to vitronectin but not laminin, and thereby inhibited vitronectin receptor (αᵥβ₃) binding to vitronectin by interfering with normal αᵥβ₃ binding activity. Other tested CTMMP-2 fusion proteins 7-1 from residues 274-637, 10-1 from residues 292-637 and 4-3 from residues 274-400 had less affect on cell adhesion compared to 2-4.

In addition to the chicken MMP-2 GST-fusion proteins described above, two human MMP-2 GST fusion proteins were produced for expressing amino acid regions 203-631 and 439-631 of the mature human MMP-2 proenzyme polypeptide. The indicated regions correspond respectively to chicken MMP-2 regions 203-637 and 445-637. Human MMP-2-GST fusion proteins were produced by PCR as described above for the chicken MMP-2-GST fusion proteins utilizing a cDNA template that encoded the entire human MMP-2 open reading frame provided by Dr. W. G. Stetler-Stevenson at the National Cancer Institute, Bethesda, Md. Upstream 5′ primer sequences were designed based upon the previously published sequence of human MMP-2 (Collier et al., J. Biol. Chem., 263:6579-6587 (1988) and to encode an introduced internal EcoRI restriction site to allow for insertion of the amplified products into the appropriate expression vector.

One upstream primer, designed to encode a human MMP-2 protein start site at position 203 after an engineered internal EcoRI endonuclease restriction site for insertion into the pGEX-1λT GST vector, had the nucleotide sequence (5′GATGAATTCTACTGCAAGTT3′ (SEQ ID NO: 43)). The 5′ and 3′ ends of the primer respectively corresponded to positions 685-704 of the human MMP-2 open reading frame sequence. Another upstream primer, designed to encode a human MMP-2 protein start site at position 439 after an engineered internal EcoRI restriction site for insertion into the pGEX-1λT GST vector, had the nucleotide sequence (5′CACTGAATTCATCTGCAAACA3′ (SEQ ID NO: 44)). The 5′ and 3′ ends of the primer respectively corresponded to positions 1392 and 1412 of the human MMP-2 open reading frame sequence.

Each of the above primers were used separately with a downstream primer, having 5′ and 3′ ends respectively complementary to bases 1998 and 1978 of the human MMP-2 sequence that ends distal to the MMP-2 open reading frame and directs protein termination after amino acid residue 631. The amplified products produced expressed fusion proteins containing human MMP-2 amino acid residues 203-631 (SEQ ID NO: 45) and 439-631 (SEQ ID NO: 18).

The resulting PCR products were purified, digested with EcoRI and repurified for ligation into a pGEX-1λT plasmid that was similarly digested and dephosphorylated prior to the ligation reaction. Cells were transformed as described above.

Other human MMP-2 fusion proteins containing amino acid residues 410-631 (SEQ ID NO: 17), 439-512 (SEQ ID NO: 19), 439-546 (SEQ ID NO: 20), 510-631 (SEQ ID NO: 21) and 543-631 (SEQ ID NO: 22) are also prepared as described above for use in the methods of this invention.

### Ex. 2B Ligand-Receptor Binding Assay

The synthetic peptides prepared in Example 1 along with the MMP-2 fusion proteins described above were further screened by measuring their ability to antagonize αᵥβ₃ and α_{IIb}β₃ receptor binding activity in purified ligand-receptor binding assays. The method for these binding studies has been described by Barbas et al., Proc. Natl. Acad. Sci., USA, 90:10003-10007 (1993), Smith et al., J. Biol. Chem., 265:11008-11013 (1990), and Pfaff et al., J. Biol. Chem., 269:20233-20238 (1994), the disclosures of which are hereby incorporated by reference.

Herein described is a method of identifying antagonists in a ligand-receptor binding assay in which the receptor is immobilized to a solid support and the ligand and antagonist are soluble. Also described is a ligand-receptor binding assay in which the ligand is immobilized to a solid support and the receptor and antagonists are soluble.

Briefly, selected purified integrins were separately immobilized in Titertek microtiter wells at a coating concentration of 50 nanograms (ng) per well. The purification of the receptors used in the ligand-receptor binding assays are well known in the art and are readily obtainable with methods familiar to one of ordinary skill in the art. After incubation for 18 hours at 4° C., nonspecific binding sites on the plate were blocked with 10 milligrams/milliliter (mg/ml) of bovine serum albumin (BSA) in Tris-buffered saline. For inhibition studies, various concentrations of selected peptides from Table 1 were tested for the ability to block the binding of ¹²⁵I-vitronectin or ¹²⁵I-fibrinogen to the integrin receptors, αᵥβ₃ and α_{IIb}β₃ Although these ligands exhibit optimal binding for a particular integrin, vitronectin for αᵥβ₃ and fibrinogen for α_{IIb}β₃, inhibition of binding studies using peptides to block the binding of fibrinogen to either receptor allowed for the accurate determination of the amount in micromoles (μM) of peptide necessary to half-maximally inhibit the binding of receptor to ligand. Radiolabeled ligands were used at concentrations of 1 nM and binding was challenged separately with unlabeled synthetic peptides.

Following a three hour incubation, free ligand was removed by washing and bound ligand was detected by gamma counting. The data from the assays where selected cyclic peptides listed in Table 1 were used to inhibit the binding of receptors and radiolabeled fibrinogen to separately immobilized αᵥβ₃ and α_{IIb}β₃ receptors were highly reproducible with the error between data points typically below 11%. The IC₅₀ data in micromoles (IC₅₀ μM) are expressed as the average of duplicate data points±the standard deviation as shown in Table 2.

TABLE 2

| Peptide No. | αᵥβ₃ (IC₅₀ μM) | α_{IIb}β₃ (IC₅₀ μM) |
|---|---|---|
| 62181 | 1.96 ± 0.62 | 14.95 ± 7.84 |
| 62184 | 0.05 ± 0.001 | 0.525 ± 0.10 |
| 62185 | 0.885 ± 0.16 | 100 ± 0.001 |
| 62187 | 0.05 ± 0.001 | 0.26 ± 0.056 |
| 62186 | 57.45 ± 7.84 | 100 ± 0.001 |
| 62175 | 1.05 ± 0.07 | 0.63 ± 0.18 |
| 62179 | 0.395 ± 0.21 | 0.055 ± 0.007 |

Thus, the RGD-containing or RGD-derivatized cyclic peptides 62181, 62184, 62185 and 62187, each having one D-amino acid residue, exhibited preferential inhibition of fibrinogen binding to the αᵥβ₃ receptor as measured by the lower concentration of peptide required for half-maximal inhibition as compared to that for the α_{IIb}β₃ receptor. In contrast, the other RGD-containing or RGD-derivatized cyclic peptides, 62186, 62175 and 62179, were either not as effective in blocking fibrinogen binding to αᵥβ₃ or exhibited preferential inhibition of fibrinogen binding to α_{IIb}β₃ as compared to αᵥβ₃. These results are consistent with those recently published by Pfaff, et al., J. Biol. Chem., 269:20233-20238 (1994) in which the cyclic peptide RGDFV (wherein F indicates a D-amino acid residue) specifically inhibited binding of fibrinogen to the αᵥβ₃ integrin and not to the α_{IIb}β₃ or α₅β₁ integrins. Similar inhibition of binding assays were performed with linearized peptides having or lacking an RGD motif, the sequences of which were derived from the αᵥ receptor subunit, α_{IIb} receptor subunit or vitronectin ligand amino acid residue sequences. The sequences of the linear peptides, 62880 (VN-derived amino acid residues 35-49), 62411 (αᵥ-derived amino acid residues 676-687); 62503 (αᵥ-derived amino acid residues 655-667) and 62502 (α_{IIb}-derived amino acid residues 296-306), are listed in Table 1. Each of these peptides were used in separate assays to inhibit the binding of either vitronectin (VN) or fibrinogen (FG) to either α_{IIb}β₃ or αᵥβ₃. The IC₅₀ data in micromoles (IC₅₀ βM) of an individual assay for each experiment is shown in Table 3.

TABLE 3

| | |
|---|---|
| α_{IIb}β₃ IC₅₀ (μM) | αᵥβ₃ IC₅₀ (μM) |

| Peptide No. | FG | VN | FG | VN |
|---|---|---|---|---|
| 62880 | 4.2 | 0.98 | <0.1 | 0.5 |
| 62411 | >100 | >100 | >100 | >100 |
| 62503 | >100 | >100 | >100 | >100 |
| 62502 | 90 | 5 | >100 | >100 |

The results of inhibition of ligand binding assays to selected integrin receptors with linearized peptides show that only peptide 62880 was effective at inhibiting the half-maximal binding of either FG or VN to αᵥβ₃ as measured by the lower concentration of peptide required for half-maximal inhibition as compared to α_{IIb}β₃ receptor. None of the other linearized peptides were effective at blocking ligand binding to αᵥβ₃ although peptide 62502 was effective at blocking VN binding to α_{IIb}β₃.

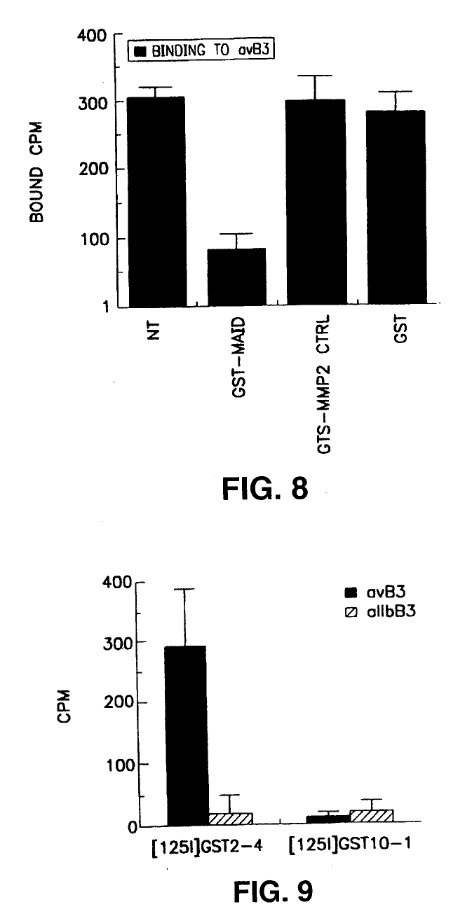

In other ligand receptor binding assays performed as described above with the exception that detection of binding or inhibition thereof was with ELISA and peroxidase-conjugated goat anti-rabbit IgG, the ligands VN, MMP-2 and fibronectin at a range of 5-50 ng/well and listed in the order of effectiveness were shown to bind to immobilized αᵥβ₃ receptor while collagen did not. In addition, the ability of peptides to inhibit the binding of either MMP-2 or VN to immobilized αᵥβ₃ was assessed with peptides 69601 (SEQ ID NO: 6) and 66203 (SEQ ID NO: 5). Only peptide 66203 was effective at inhibiting the binding of either substrate to the αᵥβ₃ receptor while the control peptide 69601 failed to have an effect with either ligand. Specificity of MMP-2 binding to integrin receptors was confirmed with a solid phase receptor binding assay in which iodinated MMP-2 was shown to bind to αᵥβ₃ and not to α_{IIb}β₃ that had been immobilized on a solid phase (300 bound cpm versus approximately 10 bound CPM). The ability of an MMP-2 derived peptide or fusion protein to inhibit the specific binding of MMP-2 to αᵥβ₃ was demonstrated in a comparable assay, the results of which are shown in FIG. 8. The GST-CTMMP-2(445-637) (also referred to as CTMMP-2(2-4)) fusion protein prepared as described above, labeled GST-MAID, inhibited the binding of iodinated MMP-2 to αᵥβ₃ while GST alone had no effect with levels of bound CPM comparable to wells receiving no inhibitor at all (labeled NT). The MMP-2 fusion protein referred to as CTMMP-2(274-637), also referred to as CTMMP-2(10-1), failed to inhibit the binding of labeled MMP-2 to αᵥβ₃.

Specificity of receptor interaction with MMP-2-derived antagonists was confirmed with binding and inhibition of binding solid phase assays. CTMMP-2(2-4), labeled in FIG. 9 as [125I]GST2-4, bound to αᵥβ₃ and not to α_{IIb}β₃ while CTMMP-2 (10-1) , labeled in FIG. 9 as [125I]GST10-1, did not bind to either receptor in the in vitro solid phase assay. In addition, the binding of labeled GST2-4 was competed by unlabeled GST2-4.

Thus, the ligand-receptor assay described herein can be used to screen for both circular or linearized synthetic peptides that exhibit selective specificity for a particular integrin receptor, specifically αᵥβ₃, as used as vitronectin receptor (αᵥβ₃) antagonists in practicing this invention.

### EXAMPLE 3

Characterization of the Untreated Chick Chorioallantoic Membrane (CAM).

### Ex. 3A Preparation of the CAM

Angiogenesis can be induced on the chick chorioallantoic membrane (CAM) after normal embryonic angiogenesis has resulted in the formation of mature blood vessels. Angiogenesis has been shown to be induced in response to specific cytokines or tumor fragments as described by Leibovich et al., Nature, 329:630 (1987) and Ausprunk et al., Am. J. Pathol., 79:597 (1975). CAMs were prepared from chick embryos for subsequent induction of angiogenesis and inhibition thereof as described in Examples 4 and 5, respectively. Ten day old chick embryos were obtained from McIntyre Poultry (Lakeside, Calif.) and incubated at 37° C. with 60% humidity. A small hole was made through the shell at the end of the egg directly over the air sac with the use of a small crafts drill (Dremel, Division of Emerson Electric Co. Racine Wis.). A second hole was drilled on the broad side of the egg in a region devoid of embryonic blood vessels determined previously by candling the egg. Negative pressure was applied to the original hole, which resulted in the CAM (chorioallantoic membrane) pulling away from the shell membrane and creating a false air sac over the CAM. A 1.0 centimeter (cm)×1.0 cm square window was cut through the shell over the dropped CAM with the use of a small model grinding wheel (Dremel). The small window allowed direct access to the underlying CAM.

The resultant CAM preparation was then either used at 6 days of embryogenesis, a stage marked by active neovascularization, without additional treatment to the CAM reflecting the model used for evaluating effects on embryonic neovascularization or used at 10 days of embryogenesis where angiogenesis has subsided. The latter preparation was thus used in this invention for inducing renewed angiogenesis in response to cytokine treatment or tumor contact as described in Example 4.

### Ex. 3B Histology of the CAM

To analyze the microscopic structure of the chick embryo CAMs and/or human tumors that were resected from the chick embryos as described in Example 6, the CAMs and tumors were prepared for frozen sectioning. Six micron (μm) thick sections were cut from the frozen blocks on a cryostat microtome for immunofluorescence analysis.

As angiogenesis in the CAM system is subsiding by this stage of embryogenesis, the system is useful in this invention for stimulating the production of new vasculature from existing vessels from adjacent areas into areas of the CAM currently lacking any vessels.

### Ex. 3C Integrin Profiles in the CAM Detected by Immunofluorescence

To view the tissue distribution of integrin receptors present in CAM tissues, 6 μm frozen sections of both tumor tissue and chick embryo CAM tissues were fixed in acetone for 30 seconds and stained by immunofluorescence with 10 micrograms/milliliter (μg/ml) mAb CSAT, a monoclonal antibody specific for the β₁ integrin subunit as described by Buck et al., J. Cell Biol., 107:2351 (1988) and thus used for controls, or αᵥβ₃ antibody LM609. Primary staining was followed by staining with a 1:250 dilution of goat anti-mouse rhodamine labeled secondary antibody (Tago) to allow for the detection of the primary immunoreaction product. The sections were then analyzed with a Zeiss immunofluorescence compound microscope.

The results of the immunofluorescence analysis show that the mature blood vessels present in an untreated 10 day chick embryo expressed the integrin β₁ subunit. In contrast, in a serial section of the tissue, no immunoreactivity with LM609 was revealed. Thus, the integrin αᵥβ₃ detected by the LM609 antibody was not actively being expressed by the mature blood vessels present in a 10 day old untreated chick embryo. As shown in the CAM model and in the following Examples, while the blood vessels are undergoing new growth in normal embryogenesis or induced by either cytokines or tumors, the blood vessels are expressing αᵥβ₃. However, following active neovascularization, once the vessels have stopped developing, the expression of αᵥβ₃ diminishes to levels not detectable by immunofluorescence analysis. This regulation of αᵥβ₃ expression in blood vessels undergoing angiogenesis as contrasted to the lack of expression in mature vessels provides for the unique ability of this invention to control and inhibit angiogenesis as shown in the following Examples using the CAM angiogenesis assay system.

In other profiles, the metalloproteinase MMP-2 and αᵥβ₃ colocalized on endothelial cells undergoing angiogenesis three days following bFGF induction in the 10 day old CAM model. MMP-2 was only minimally expressed on vessels that lacked the αᵥβ₃ receptor. In addition, MMP-2 colocalized with αᵥβ₃ on angiogenic M21-L tumor-associated blood vessels in vivo (tumors resulting from injection of M21-L human melanoma cells into the dermis of human skin grafts grown on SCID mice as described in Example 9) but not with preexisting non-tumor associated blood vessels. Similar results of the selective association of MMP-2 and αᵥβ₃ were also obtained with αᵥβ₃ bearing CS-1 melanoma tumors in the CAM model but not with CS-1 cells lacking αᵥβ₃.

### EXAMPLE 4

CAM Angiogenesis Assay.

### Ex. 4A Angiogenesis Induced by Growth Factors

Angiogenesis has been shown to be induced by cytokines or growth factors as referenced in Example 3A. In the experiments described herein, angiogenesis in the CAM preparation described in Example 3 was induced by growth factors that were topically applied onto the CAM blood vessels as described herein.

Angiogenesis was induced by placing a 5 millimeter (mm)×5 mm Whatman filter disk (Whatman Filter paper No.1) saturated with Hanks Balanced Salt Solution (HBSS, GIBCO, Grand Island, N.Y.) or HBSS containing 150 nanograms/milliliter (ng/ml) recombinant basic fibroblast growth factor (bFGF) (Genzyme, Cambridge, Mass.) on the CAM of a 10-day chick embryo in a region devoid of blood vessels and the windows were latter sealed with tape. In other assays, 125 ng/ml bFGF was also effective at inducing blood vessel growth. For assays where inhibition of angiogenesis ws evaluated with intravenous injections of antagonists, angiogenesis was first induced with 1-2 μg/ml bFGF in fibroblast growth medium. Angiogenesis was monitored by photomicroscopy after 72 hours. CAMs were snap frozen, and 6 um cryostat sections were fixed with acetone and stained by immunofluorescence as described in Example 3C with 10 μg/ml of either anti-β₁ monoclonal antibody CSAT or LM609.

Immunofluorescence photomicrographic analysis indicated enhanced expression of αᵥβ₃ during bFGF-induced angiogenesis on the chick CAM in contrast with the absence of αᵥβ₃ expression in an untreated chick CAM. αᵥβ₃ was readily detectable on many (75% to 80%) of the vessels on the bFGF-treated CAMs. In addition, the expression of integrin β₁ did not change from that seen in an untreated CAM as β₁ was also readily detectable on stimulated blood vessels.

The relative expression of αᵥβ₃ and β₁ integrins was then quantified during bFGF-induced angiogenesis by laser confocal image analysis of the CAM cryostat sections. The stained sections were then analyzed with a Zeiss laser confocal microscope. Twenty-five vessels stained with LM609 and 15 stained with CSAT (average size about 1200 mm², range 350 to 3, 500 mm²) were selected from random fields and the average rhodamine fluorescence for each vessel per unit area was measured in arbitrary units by laser confocal image analysis. Data are expressed as the mean fluorescence intensity in arbitrary units of vessels±standard error (SE).

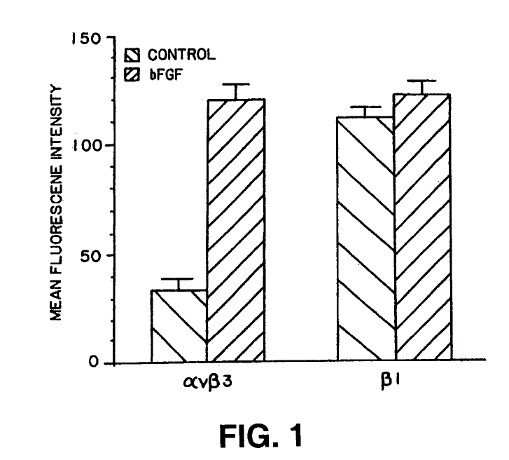

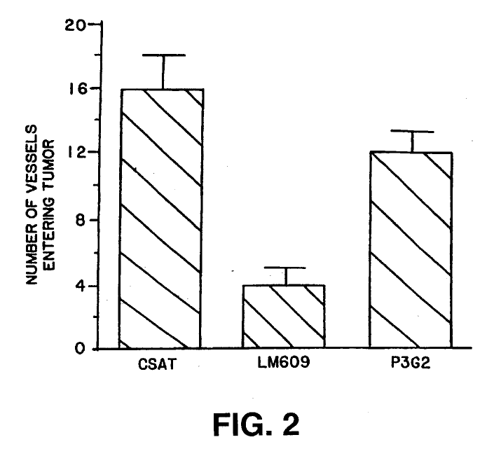

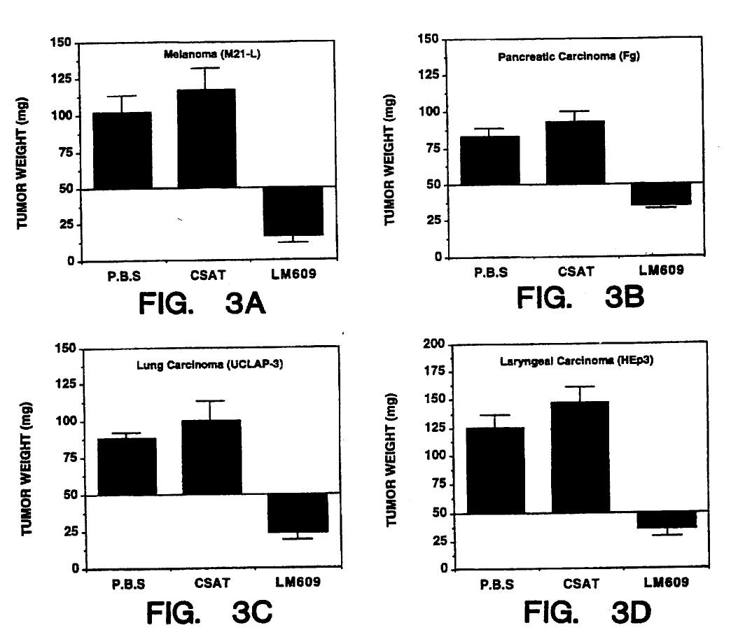

The results plotted in FIG. 1 show that staining of αᵥβ₃ was significantly enhanced (four times higher) on CAMs treated with bFGF as determined by the Wilcoxon Rank Sum Test (P<0.0001) whereas β₁ staining was not significantly different with bFGF treatment.

The CAM assay was further used to examine the effect of another potent angiogenesis inducer, tumor necrosis factor-alpha (TNFα), on the expression of β₁ and β₃ integrins. Filter disks impregnated with either bFGF or TNFα and placed on CAMs from 10 day embryos were found to promote local angiogenesis after 72 hours.

Blood vessels were readily apparent in both the bFGF and TNFα treated preparations but are not present in the untreated CAM. Thus, the topical application of a growth factor/cytokine resulted in the induction of angiogenesis from mature vessels in an adjacent area into the area originally devoid of blood vessels. In view of the bFGF-induced blood vessels and concomitant expression of αᵥβ₃, treatment of TNFα results in comparable activities.

These findings indicate that in both human and chick, blood vessels involved in angiogenesis show enhanced expression of αᵥβ₃. Consistent with this, expression of αᵥβ₃ on cultured endothelial cells can be induced by various cytokines in vitro as described by Janat et al., J. Cell Physiol., 151:588 (1992); Enenstein et al., Exp. Cell Res., 203:499 (1992) and Swerlick et al., J. Invest. Derm., 99:715 (1993).

The effect on growth-factor induced angiogenesis by antibody and peptide inhibitors is presented in Examples 5A and 5B.

### Ex. 4B Embryonic Angiogenesis

The CAM preparation for evaluating the effect of angiogenesis inhibitors on the natural formation of embryonic neovasculature was the 6 day chick embryo as previously described. At this stage in development, the blood vessels are undergoing de novo growth and thus provides a useful system for determining if αᵥβ₃ participates in embryonic angiogenesis. The CAM system was prepared as described above with the exception that the assay was performed at embryonic day 6 rather than at day 10. The effect on embryonic angiogenesis by treatment with antibodies and peptides of this invention are presented in Example 5C.

### Ex. 4C Angiogenesis Induced by Tumors

To investigate the role of αᵥβ₃ in tumor-induced angiogenesis, various αᵥβ₃-negative human melanoma and carcinoma fragments were used in the CAM assay that were previously grown and isolated from the CAM of 17-day chick embryo as described by Brooks et al., J. Cell Biol., 122:1351 (1993) and as described herein. The fragments induced extensive neovascularization in the presence of buffer alone.

Angiogenesis was induced in the CAM assay system by direct apposition of a tumor fragment on the CAM. Preparation of the chick embryo CAM was identical to the procedure described above. Instead of a filter paper disk, a 50 milligram (mg) to 55 mg in weight fragment of one of human melanoma tumor M21-L, human lung carcinoma tumor UCLAP-3, human pancreatic carcinoma cell line FG (Cheresh et al., Cell 58:945-953, 1989), or human laryngeal carcinoma cell line HEp3, all of which are αᵥβ₃ negative tumors, was placed on the CAM in an area originally devoid of blood vessels.

The M21-L human melanoma cell line, UCLAP-3 human lung carcinoma cell line, FG pancreatic carcinoma cell line, or HEp3 human laryngeal carcinoma cell line, all αᵥβ₃ negative, were used to grow the solid human tumors on the CAMs of chick embryos. A single cell suspension of 8×10⁶ M21-L, UCLAP-3, and FB or 5×10⁵ HEp3 cells was first applied to the CAMs in a total volume of 30 μl of sterile HBSS. The windows were sealed with tape and the embryos were incubated for 7 days to allow growth of human tumor lesions. At the end of 7 days, now a 17-day embryo, the tumors were resected from the CAMs and trimmed free of surrounding CAM tissue. The tumors were sliced into 50 mg to 55 mg tumor fragments for use in either angiogenesis or tumor growth assays. The tumor fragments were placed on a new set of 10 day chick embryo CAMs as described in Example 4A in an area devoid of blood vessels.

Tumors grown in vivo on the chick embryo CAMs were stained for αᵥβ₃ expression with mAb LM609. No specific staining of tumor cells was observed indicating a lack of αᵥβ₃ expression.

These CAM tumor preparations were then subsequently treated as described in Example 5 for measuring the effects of antibodies and peptides on tumor-induced angiogenesis.

### EXAMPLE 5

Inhibition of Angiogenesis as Measured in the CAM Assay

### Ex. 5A Inhibition of Growth Factor-Induced Angiogenesis by Topical Application of Inhibitors

### Ex. 5A(1) Treatment with Synthetic Peptides

CAM assays were also performed with the synthetic peptides of this invention to determine the effect of cyclic and linearized peptides on growth factor induced angiogenesis. The peptides were prepared as described in Example 1 and 80 μg of peptide were presented in a total volume of 25 μl of sterile HBSS. The peptide solution was applied to the CAM preparation immediately and then again at 24 and 48 hrs. At 72 hours the filter paper and surrounding CAM tissue was dissected and viewed as described above.

Results from this assay revealed were similar to those where synthetic peptides were intravenously injected into tumor induced blood vessels. Here, with the control peptide, 62186, the bFGF-induced blood vessels remained undisturbed. In contrast when the cyclic RGD peptide, 62184, was applied to the filter, the formation of blood vessels was inhibited leaving the area devoid of new vasculature. In addition, in areas in which mature blood vessels were present yet distant from the placement of the growth-factor saturated filter, no effect was seen with the topical treatment of synthetic peptides on these outlying vessels. The inhibitory activity of the peptides on angiogenesis thus is limited to the areas of angiogenesis induced by growth factors and does not effect adjacent preexisting mature vessels or result in any deleterious cytotoxicity to the surrounding area.

Similar assays are performed with the other synthetic peptides prepared in Example 1 and listed in Table 1.

### Ex. 5A(2) Treatment with MMP-2 Peptide Fragments

To demonstrate the biological effects of MMP-2 peptide fragments on angiogenesis, CAM assays were performed as described above with the exception that angiogenesis was induced with filter discs saturated for 10 minutes with bFGF at a concentration of 1.0 ug/ml in HBS. The discs were then positioned on the CAM in an area that was reduced in the number of preexisting vessels. The C-terminal CTMMP-2(410-637) fusion protein, prepared as described above, or control GST receptor associated fusion protein (RAP) (1.5 μg in 30 μl of HBSS) was applied then topically to the filter disc once per day for a total of three days. At the end of the incubation period, the embryos were sacrificed and the filter disc and underlying CAM tissue was resected and analyzed for angiogenesis with a stereo microscope. Angiogenesis was quantified by counting the number of blood vessels branch points that occur within the confines of the filter discs. The branched blood vessels are considered to correspond primarily to new angiogenic sprouting blood vessels.

Quantification was performed in a double blind manner by at least two independent observers. The results are expressed as the Angiogenic Index where the angiogenic index is the number of branch points (bFGF stimulated) minus the number of branch points (control unstimulated) per filter disc. Experiments routinely had 6-10 embryos per condition.

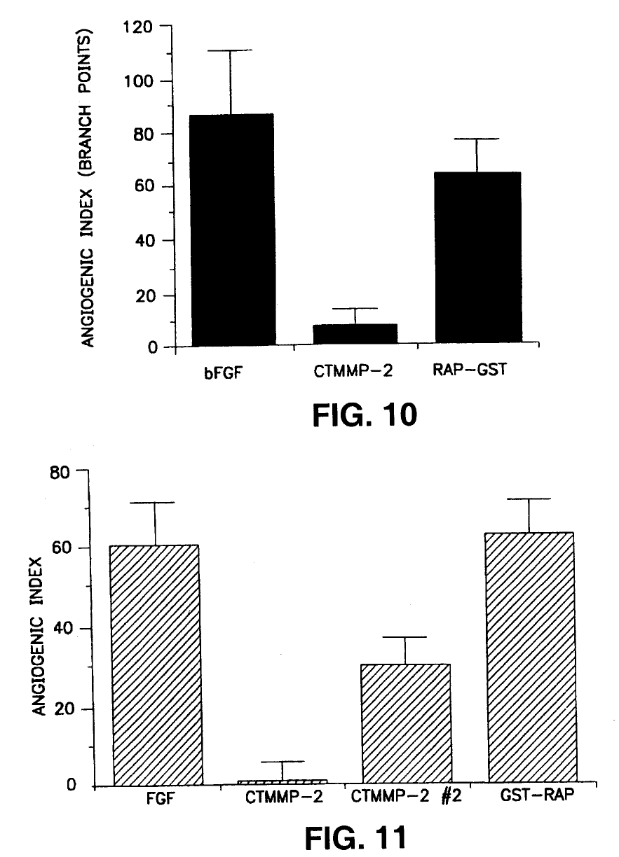

The results of the CAM angiogenesis assay are shown in FIGS. 10 and 11. FIGS. 10 and 11 are bar graphs illustrating the angiogenesis index of CAM angiogenesis assays with CTMMP-2, the same fusion protein as above, compared to controls (bFGF only or GST-RAP fusion protein). In FIG. 11, the results of two separate evaluations (#1 & #2) using CTMMP-2(410-637) fusion protein are shown.

These results demonstrated in FIGS. 10 and 11 indicate that a CTMMP-2 fusion protein or polypeptide containing a C-terminal domain of MMP-2 is a useful composition for inhibition of bFGF-mediated angiogenesis by inhibiting αᵥβ₃.

### Ex. 5B Inhibition of Growth Factor-Induced Angiogenesis by Intravenous Application of Inhibitors

### Ex. 5B(1) Treatment with Synthetic Peptides

For CAM preparations in which angiogenesis was induced with 1-2 μg/ml bFGF as previously described, synthetic peptides 69601 (control) and 66203 (SEQ ID NO: 5) were separately intravenously injected into CAM preparations 18 hours after bFGF induction of angiogenesis. The preparations were maintained for an additional 36-40 hours after which time the number of branch points were determined as previously described.

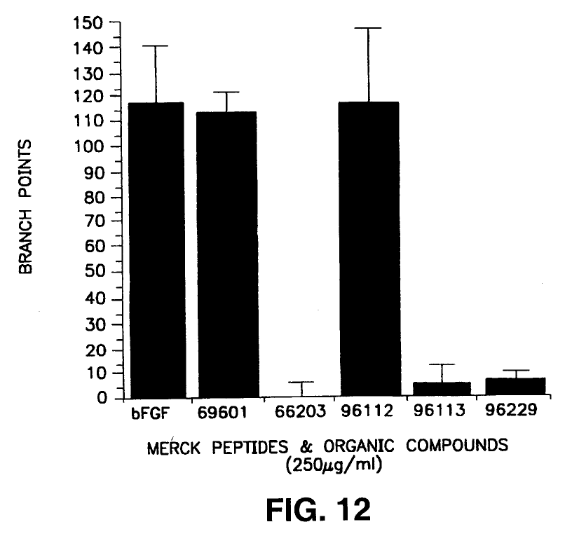

The results are shown in FIG. 12 where peptide 66203 completely inhibited bFGF-induced angiogenesis in contrast to the absence of inhibition with the control peptide.

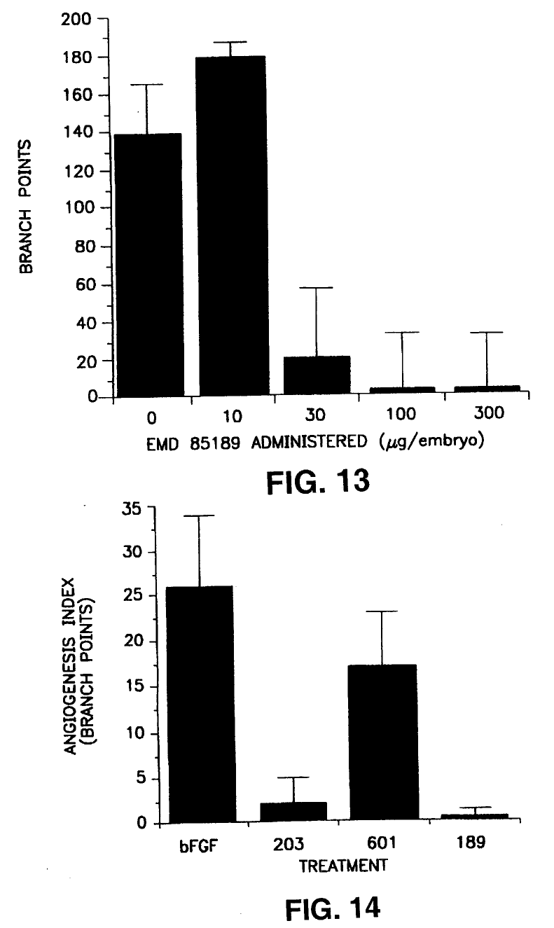

In other assays, peptide 85189 (SEQ ID NO: 15) was evaluated for inhibiting bFGF-induced angiogenesis in the CAM assay over a dosage range of 10 μg/embryo to 300 μg/embryo. The assay was performed as previously described. The results are shown in FIG. 13 where the lowest effective dose was 30 ug with 100 and 300 μg nearly completely inhibiting angiogenesis.

In still further assays, peptide 85189 was compared to peptides 69601 and 66203 for anti-angiogenesis activity. The assay was performed as described above with the exception that 50 μg peptide were used. The results, plotted in FIG. 14, showed that peptides 66203 (labeled 203) and 85189 (labeled 189) were effective inhibitors of bFGF-mediated angiogenesis compared to bFGF-treated (labeled bFGF) and 69601-treated (labeled 601) controls.

The effectiveness of the different salt formulations of peptide 85189 was also evaluated in similar bFGF-induced CAM assays. The peptides were used at 100 μg/embryo. The same peptide sequence in HCl (peptide 85189) and in TFA (peptide 121974) inhibited bFGF-induced angiogenesis with the HCl formulated peptide being slightly more effective than that in TFA (the respective number of branch points for peptide 85189 versus 121974 is 30 versus 60). Untreated CAMs, labeled as “no cytokine” had approximately half as many branch points as that seen with bFGF treatment, respectively 70 versus 190. Treatment with control peptide 69601 had no effect on inhibiting angiogenesis (230 branch points).

The other synthetic peptides prepared in Example 1 are separately intravenously injected into the growth factor induced blood vessels in the CAM preparation as described above. The effect of the peptides on the viability of the vessels is similarly assessed.

### Ex. 5B(2) Treatment with MMP-2 Fragments

With the above-described protocol, the effect of MMP-2 fusion proteins, CTMMP-2(2-4), also referred to as CTMMP-2(445-467) and CTMMP-2(10-1), also referred to as CTMMP-2(274-637) was also evaluated. The assay was performed as previously described with the exception that 50 μg of fusion protein was administered to the bFGF-treated embryos. The effect of fusion protein treatment was assessed at 24 hours, 48 hours and 72 hours.

The significant induction of angiogenesis after 48 and 72 hours following bFGF treatment was almost completely inhibited only with exposure to CTMMP-2(2-4). The extent of inhibition with CTMMP-2(2-4) was greater than that seen with CTMMP-2(10-1) which exhibited some in vivo anti-angiogenesis activity.

The other MMP-2 compositions, whole MMP-2, fragments and fusion proteins, prepared as previously described are also separately intravenously injected into the growth factor induced blood vessels in the CAM preparation as described above. The effect of the peptides on the viability of the vessels is similarly assessed.

### Ex. 5C Inhibition of Tumor-Induced Angiogenesis by Intravenous Application

### Ex. 5C(1) Treatment with Synthetic Peptides

The effects of peptide exposure to tumor-induced vasculature in the CAM assay system was also assessed. The tumor-CAM preparation was used as described above with the exception that instead of intravenous injection of a mAb, synthetic peptides prepared as described in Example 1 and Example 5A(1) were separately intravenously injected into visible blood vessels.

The treatment with the control peptide did not effect the abundant large blood vessels that were induced by the tumor treatment to grow into an area originally devoid of blood vessels of the CAM. In contrast when the cyclic RGD peptide, 66203, an antagonist to αᵥβ₃, was applied to the filter, the formation of blood vessels was inhibited leaving the area devoid of new vasculature. The inhibitory effect of the RGD-containing peptide was specific and localized as evidenced by an absence of any deleterious effects to vessels located adjacent to the tumor placement. Thus, when inhibitory peptides are intravenously injected into the CAM assay system, no effect was seen on the preexisting mature vessels present in the CAM in areas adjacent yet distant from the placement of the tumor. The preexisting vessels in this location were not affected by the inhibitory peptide that flowed within those vessels although the generation of new vessels from these preexisting vessels into the tumor mass was inhibited. Thus, synthetic peptides including 66203 and 62184, previously shown in ligand-receptor assays in Example 2 to be antagonists of αᵥβ₃, have now been demonstrated to inhibit angiogenesis that is limited to vessels undergoing development and not to mature preexisting vessels. In addition, the intravenous infusion of peptides does not result in any deleterious cytotoxicity to the surrounding area as evidence by the intact vasculature.

Similar assays are performed with the other synthetic peptides prepared in Example 1 and listed in Table 1 along with the MMP-2 compositions of this invention.

### Ex. 5C(2) Treatment with MMP-2 Fragments

A CS-1 tumor (β₃-negative) was prepared in a CAM as described above. After 24 hours of tumor growth, a composition of MMP-2 fragment, designated CTMMP-2(2-4) and prepared as described in Example 2A, was administered intraveneously at 50 μg fragment in 100 μl of PBS. After 6 days, the tumor was evaluated for mass. Tumors treated with CTMMP-2(2-4) were reduced in growth rate by about 50% when compared to the growth rate of control tumors treated with CTMMP-2(10-1) or with PBS control. Thus, the αᵥβ₃ antagonist inhibited tumor growth.

### EXAMPLE 6

Inhibition of Tumor Tissue Growth With αᵥβ₃ Antagonists As Measured in the CAM Assay.

As described in Example 5, in addition to visually assessing the effect of anti-αᵥβ₃ antagonists on growth factor or tumor induced angiogenesis, the effect of the antagonists was also assessed by measuring any changes to the tumor mass following exposure. For this analysis, the tumor-induced angiogenesis CAM assay system was prepared as described in Example 4C. At the end of the 7 day incubation period, the resulting tumors were resected from the CAMs and trimmed free of any residual CAM tissue, washed with 1 ml of phosphate buffer saline and wet weights were determined for each tumor.

In addition, preparation of the tumor for microscopic histological analysis included fixing representative examples of tumors in Bulins Fixative for 8 hours and embedding in paraffin. Serial sections were cut and stained with hematoxylin and eosin (H&E) for microscopic analysis. Gladson, et al., J. Clin. Invest., 88:1924 (1991). Sections were photographed with an Olympus compound microscope at 250×.

### EXAMPLE 7

Regression of Tumor Tissue Growth with αᵥβ₃ Antagonists as Measured in the CAM Assay.

To further assess the effects of αᵥβ₃ antagonists on tumor growth and survival, fragments of human melanoma and fragments of carcinomas of the lung, pancreas, and larynx were placed on CAMS of 10-day old embryos as described in Example 3A.

Human M21-L melanoma tumor fragments (50 mg) were implanted on the CAMs of 10 day old embryos as described in Example 3A and 3C. Twenty four hours later, embryos received a single intravenous injection of 300 μg/100 μl of either the cyclo-RADfV (69601) and or cyclo-RGDfV (66203). After a total of 72 hours, tumors were removed, examined morphologically, and photographed with a stereo microscope.

Only peptide 66203 in contrast to control peptide 69601 inhibited vessel formation. Vessels in the CAM tissue adjacent to the tumor were not affected.

Additional tumor regression assays were performed with the αᵥβ₃-reactive peptide 85189 (SEQ ID NO: 15) against 69601 as a control. The assays were performed as described above with the exception that 100 μg of peptide was intravenously injected into the CAM at 18 hours post-implantation. After 48 hours more, the tumors were then resected and wet weights were obtained.

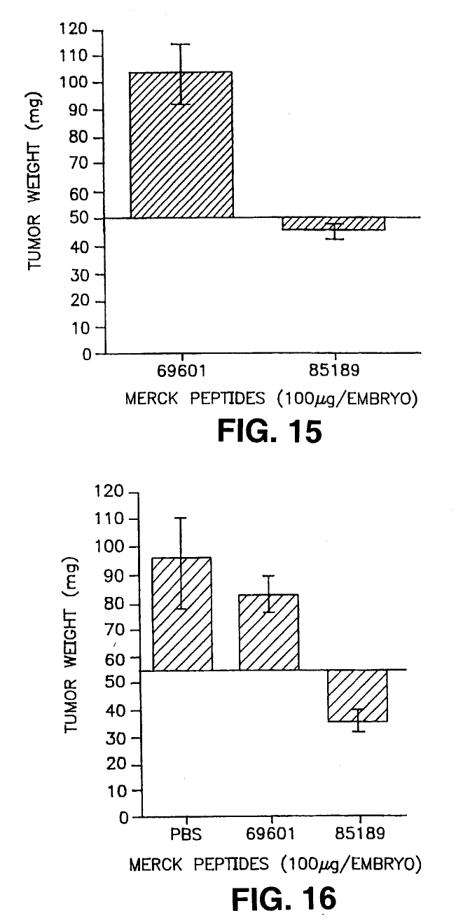

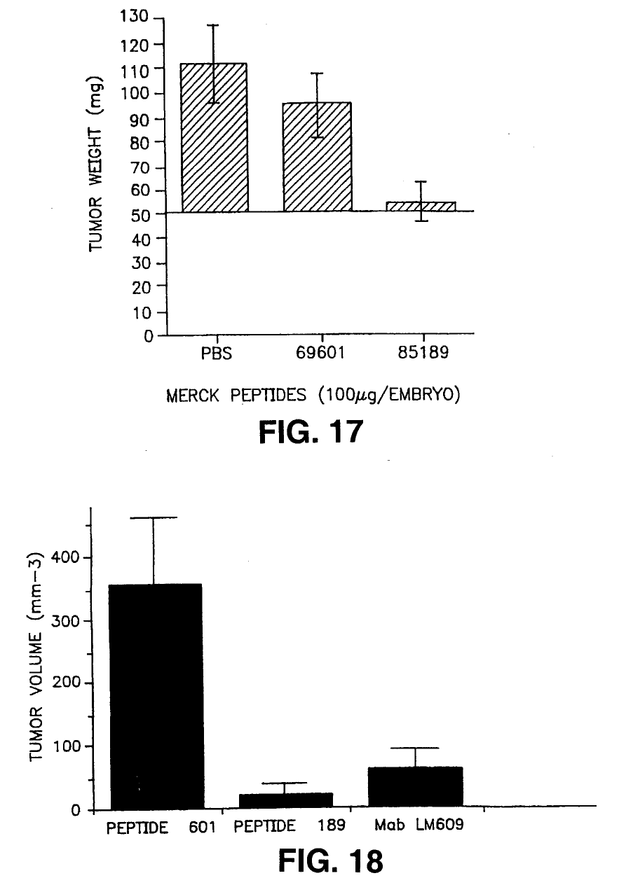

FIGS. 15, 16 and 17 respectively show the reduction in tumor weight for UCLAP-3, M21-L and FgM tumors following intravenous exposure to peptide 85189 in contrast to the lack of effect with either PBS or peptide 69601.

### EXAMPLE 8

Regression of Tumor Tissue Growth with αᵥβ₃ Antagonists as Measured by In Vivo Rabbit Eye Model Assay.

The effect of anti-αᵥβ₃ antagonists on growth factor-induced angiogenesis can be observed in naturally transparent structures as exemplified by the cornea of the eye. New blood vessels grow from the rim of the cornea, which has a rich blood supply, toward the center of the cornea, which normally does not have a blood supply. Stimulators of angiogenesis, such as bFGF, when applied to the cornea induce the growth of new blood vessels from the rim of the cornea. Antagonists of angiogenesis, applied to the cornea, inhibit the growth of new blood vessels from the rim of the cornea. Thus, the cornea undergoes angiogenesis through an invasion of endothelial cells from the rim of the cornea into the tough collagen-packed corneal tissue which is easily visible. The rabbit eye model assay therefore provides an in vivo model for the direct observation of stimulation and inhibition of angiogenesis following the implantation of compounds directly into the cornea of the eye.

### Ex. 8A(1) In Vivo Rabbit Eye Model Assay Angiogenesis Induced by Growth Factors

Angiogenesis was induced in the in vivo rabbit eye model assay with the growth factor bFGF and is described in the following sections.

### Ex. 8A(2) Treatment with Polypeptides

Each experiment consisted of eight rabbits in which one eye received a pellet comprising 100 nanograms (ng) bFGF and the other eye received a pellet comprising 1 microgram (μg) VEGF. The pellets were inserted into the corneal pocket as described above, and the cytokines subsequently stimulated the growth of new blood vessels into the cornea. Peptides were administered subcutaneously (s.q.) in 1 ml PBS at an initial dosage of 50 μg per kg rabbit the day of pellet insertion, and daily s.q. dosages were given at 20 μg/kg thereafter. After 7 days, the cornea were evaluated as described above.

Rabbits receiving control peptide 69601 showed substantial corneal blood vessel growth at 7 days, in both vFGF and VEGF stimulated eyes. Rabbits receiving peptide 85189 showed less than 50% of the amount of corneal blood vessel growth compared to controls in vFGF-stimulated eyes and nearly 100% inhibition in VEGF-stimulated eyes.

### EXAMPLE 9

In Vivo Regression of Tumor Tissue Growth With αᵥβ₃ Antagonists as Measured by Chimeric Mouse:Human Assay.

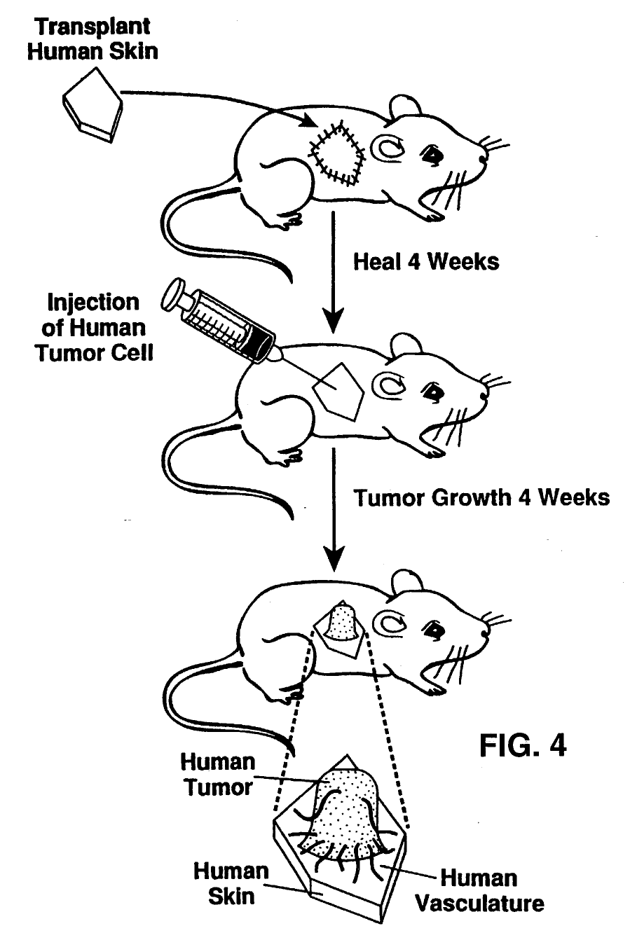

An in vivo chimeric mouse:human model was generated by replacing a portion of skin from a SCID mouse with human neonatal foreskin (FIG. 4). After the skin graft was established, the human foreskin was inoculated with carcinoma cells. After a measurable tumor was established, either an αᵥβ₃ antagonist or PBS was injected into the mouse tail vein. Following a 2-3 week period, the tumor was excised and analyzed by weight and histology.

### Ex. 9A In Vivo Chimeric Mouse:Human Assay

The in vivo chimeric mouse:human model is prepared essentially as described in Yan, et al., J. Clin. Invest., 91:986-996 (1993). Briefly, a 2 cm² square area of skin was surgically removed from a SCID mouse (6-8 weeks of age) and replaced with a human foreskin. The mouse was anesthetized and the hair removed from a 5 cm² area on each side of the lateral abdominal region by shaving. Two circular graft beds of 2 cm² were prepared by removing the full thickness of skin down to the fascia. Full thickness human skin grafts of the same size derived from human neonatal foreskin were placed onto the wound beds and sutured into place. The graft was covered with a Band-Aid which was sutured to the skin. Micropore cloth tape was also applied to cover the wound.

The M21-L human melanoma cell line or MDA 23.1 breast carcinoma cell line (ATCC HTB 26; αᵥβ₃ negative by immunoreactivity of tissue sections with mAb LM609), were used to form the solid human tumors on the human skin grafts on the SCID mice. A single cell suspension of 5×10⁶ M21-L or MDA 23.1 cells was injected intradermally into the human skin graft. The mice were then observed for 2 to 4 weeks to allow growth of measurable human tumors.

### Ex. 9B Intravenous Application

In experiments with M21-L melanoma tumor cells in the mouse:human chimeric assay system, the response with mAB LM609 was compared with the response obtained with the synthetic peptide 85189 (SEQ ID NO: 15) as compared to control synthetic peptide 69601 (SEQ ID NO: 6). The assays were performed as described above. The results, shown in FIG. 18, demonstrate that the synthetic peptide 85189 reduced tumor volume to below 25 mm³ as compared to control peptide where the tumor volume was approximately 360 mm³. The mAB LM609 also significantly reduced tumor volume to approximately 60 mm³.

Tumors formed in skin grafts which had been injected with MDA 23.1 cells were detectable and measurable. Morphological examination of the established tumors revealed that neovascularization from the grafted human tissue into the MDA 23.1 tumor cells had occurred.

Thus, blocking of the αᵥβ₃ receptor by the intravenous application of αᵥβ₃-specific LM609 antibody and peptides resulted in a regression of a carcinoma in this model system in the same manner as the CAM and rabbit eye model systems as described in Examples 7 and 8, respectively.

### Ex. 9B(1) Treatment with Synthetic Peptides

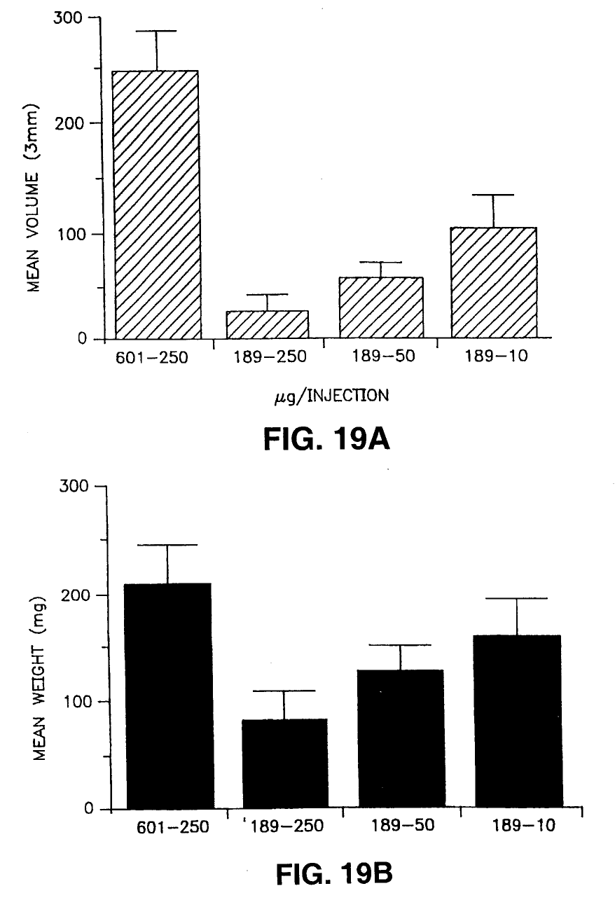

In a procedure similar to that described above for monoclonal antibodies, peptide antagonists of αᵥβ₃ were injected intravenously into the tail vein of SCID mice having measurable M21-L tumors. In a preliminary analysis, a dose response curve was performed for peptides 69601 (control) and 85189 (test) injected over a concentration range of 10 to 250 μg/ml. The mean volume and weight of resected tumors following treatment were determined with the results respectively shown in FIGS. 19A and 19B. Peptide 85189 was effective at inhibiting M21-L tumor growth over the concentration range tested compared to treatment with control peptide with the most effective dosage being 250 μg/ml.

For analyzing peptide 85189 treatment effectiveness over a time course, two treatment regimens were evaluated in the same SCID tumor model. In one assay, treatment with either peptide 85189 or 69601 was initiated on day 6, with day 0 being the day of M21-L tumor injection of 3×10⁶ cells subcutaneously into mouse skin, with intraperitoneal injections of 250 μg/ml peptide 85189 or control 69601 every other day until day 29. The other assay was identically performed with the exception that treatment was initiated on day 20. At the end of the assays, the tumors were resected and the mean tumor volume in mm³ was determined. The data was plotted as this value ± the standard error of the mean.

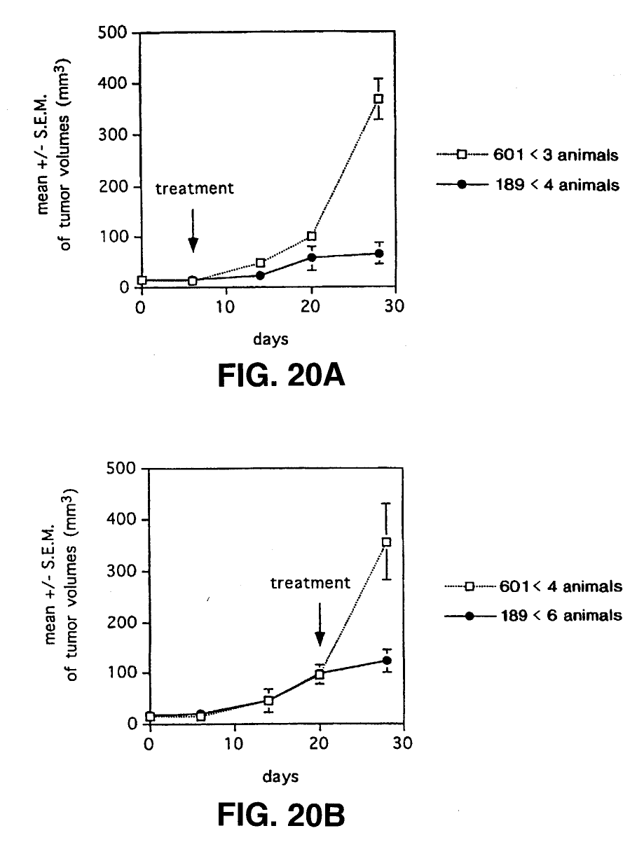

The results of these assays, respectively shown in FIGS. 20A and 20B, indicate that peptide 85189 but not 69601 inhibited tumor growth at various days after treatment was initiated, depending on the particular treatment regimen. Thus, peptide 85189 is an effective αᵥβ₃ antagonist of both angiogenesis and thus tumor growth.

### EXAMPLE 10

Stimulation of Vascular Cells to Enter the Cell Cycle and Undergo Apoptosis in the Presence of Antagonists of Integrin αᵥβ₃ as Measured in the CAM Assay.

The angiogenic process clearly depends on the capacity of cytokines such as bFGF and VEGF to stimulate vascular cell proliferation. Mignatti et al., J. Cell. Biochem., 471:201 (1991); Takeshita et al., J. Clin. Invest., 93:662 (1994); and Koyama et al., J. Cell. Physiol., 158:1 (1994). However, it is also apparent that signaling events may regulate the differentiation of these vascular cells into mature blood vessels. Thus, it is conceivable that interfering with signals related to either growth or differentiation of vascular cells undergoing new growth or angiogenesis may result in the perturbation of angiogenesis.

Integrin ligation events have been shown to participate in both cell proliferation as well as apoptosis or programmed cell death in vitro. Schwartz, Cancer Res., 51:1503 (1993); Meredith et al., Mol. Biol. Cell., 4:953 (1993); Frisch et al., J. Cell Biol., 124:619 (1994); and Ruoslahti et al., Cell, 77:477 (1994). Close examination of the effects of αᵥβ₃ antagonists on angiogenesis reveals the presence of discontinuous and disrupted tumor-associated blood vessels. Therefore, it is possible that the loss of blood vessel continuity may be due to selective necrosis or apoptosis of vascular cells.

To explore this possibility, CAMs were examined after induction of angiogenesis with the growth factor bFGF and treatment with the mAb and cyclic peptides of this invention.

### Ex. 10A Treatment with Synthetic Peptides

CAM assays with growth factor-induced angiogenesis, as described in Example 4A, were also performed with the synthetic peptides of this invention to determine the effect of cyclic peptides on apoptosis. The peptides cyclo-RGDfV (66203) and cyclo-RADfV (69601) were prepared as described in Example 1. The peptide solutions or PBS were injected into the CAM preparation at a concentration of 300 μg/ml. At 24 and 48 hours, the filter paper and surrounding CAM tissue was dissected and stained with the Apop Tag to detect apoptosis.

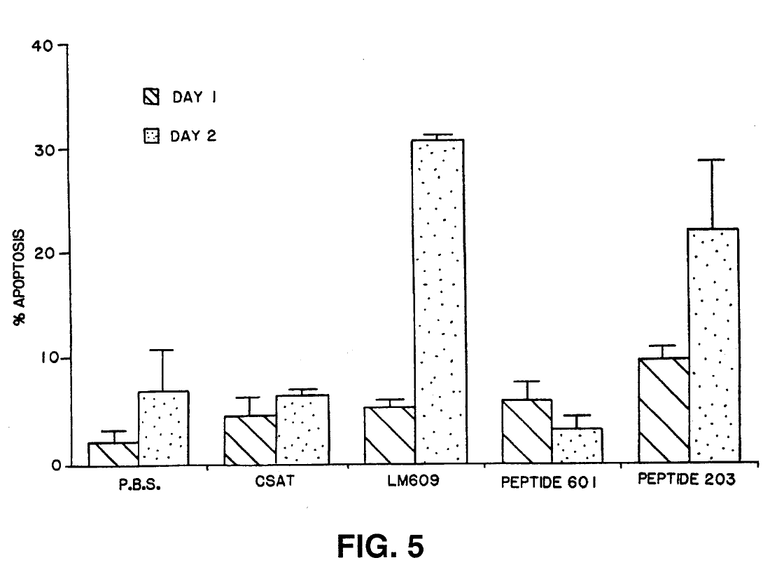

CAMs treated two days prior with peptide 69203 (cyclo-RGDfV) showed a 3 to 4-fold increase in Apop Tag staining as compared to CAMs treated with either PBS alone or control cyclic peptide 69601 (cyclo-RADfV) as shown in FIG. 5.

### EXAMPLE 11

Preparation of Organic Molecule αᵥβ₃ Antagonists.

Organic αᵥβ₃ antagonists useful in the methods of the present invention can be synthesized by methods well known in the organic chemical arts. For example, compounds of general formula (I) including compounds I(a) through I(r) can be synthesized by the methods disclosed in U.S. Pat. No. 6,204,280 to Gante et al., U.S. patent application Ser. No. 2001/002709A1, Canadian Patent Application No. 2,241,149P to Diefenbach, et al., PCT Publication No. WO 01/58893 to Goodman et al., PCT Publication No. WO 00/26212 to Fittschen et al., and European Patent No. 0964856B1, to Diefenbach et al., the relevant disclosures of which are incorporated herein by reference.

The syntheses of organic αᵥβ₃ antagonist Compounds 7 (96112), 9 (99799), 10 (96229), 12 (112854), 14 (96113), 15 (79959), 16 (81218), 17 (87292) and 18 (87293) are described below and are also shown in the noted FIGS. Organic antagonists are also referred to by the numbers in parentheses. The resultant organic molecules, referred to as organic mimetics or peptidomimetics as previously defined, are then used in the methods for inhibiting αᵥβ₃-mediated angiogenesis as described in Example 9.

For each of the syntheses described below, optical rotations were measured on Perkin-Elmer 241 spectrophotometer UV and visible spectra were recorded on a Beckmann DU-70 spectrometer. ¹H and ¹³C NMR spectra were recorded at 400 and 500 MHz on Bruker AMX-400 and AMX-500 spectrometer. High-resolution mass spectra (HRMS) were recorded on a VG ZAB-ZSE mass spectrometer under fast atom bombardment (FAB) conditions. Column chromatography was carried out with silica gel of 70-230 mesh. Preparative TLC was carried out on Merck Art. 5744 (0.5 mm). Melting points were taken on a Thomas Hoover apparatus.

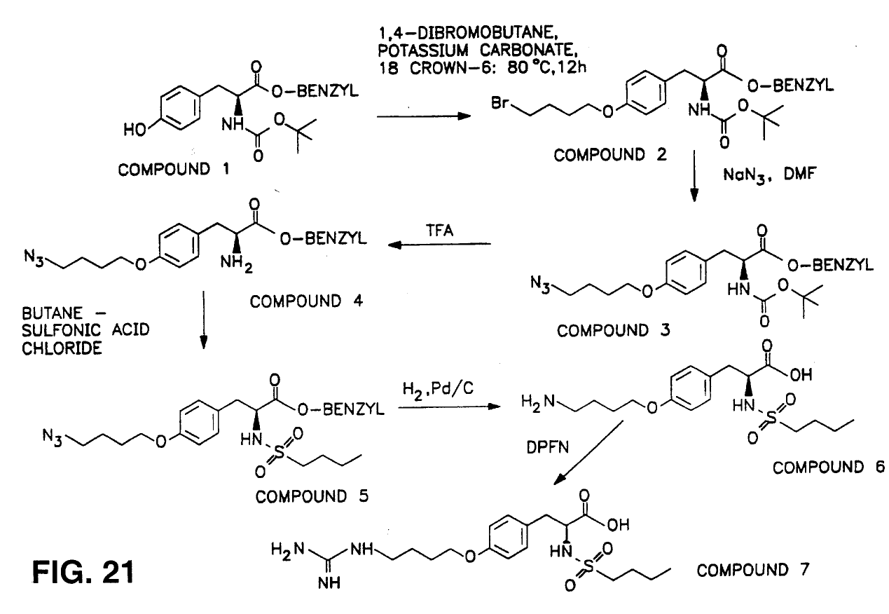

A. Compound 1: t-Boc-L-tyrosine benzyl ester as Illustrated in FIG. 21.

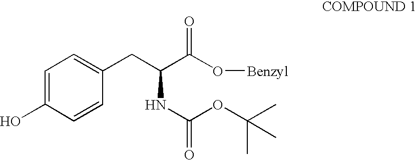

To a solution of N-(tert-butoxycarbonyl)-L-tyrosine(t-Boc-L-tyrosine) (1.0 equivalents; Aldrich) in 0.10 M (M) methylene chloride was added dicyclohexylcarbodiimide (DCC) (1.5 equivalents) at 25° C. and allowed to stir for 1 hour. Next, 1.5 equivalents benzyl alcohol was added and the mixture was stirred for an additional 12 hours at 25° C. The reaction mixture was then diluted with ethyl acetate (0.10 M) and washed twice (2×) with water, once (1×) with brine and dried over magnesium sulfate. The solvent was then removed in vacuo and the crude product was then purified by silica gel column chromatography. Compound 1, t-Boc-L-tyrosine benzyl ester can also be commercially purchased from Sigma.

B. Compound 2: (S)-3-(4-(4-Bromobutyloxy)phenyl-2-N-tert-butyloxycarbonyl-propionic acid benzyl ester as Illustrated in FIG. 21 Step i.

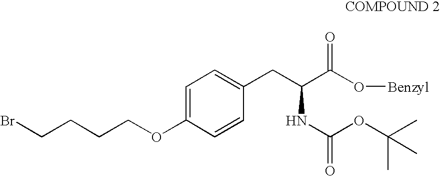

A mixture of t-Boc-L-tyrosine benzyl ester (2 grams, 5.38 mmol; synthesized as described above), 1, 4-dibromobutane (1.9 ml, 16.2 mmol; Aldrich), potassium carbonate (5 g) and 18-crown-6 (0.1 g; Aldrich), was heated at 80° C. for 12 hours. After cooling, the precipate was filtered off and the reaction mixture was evaporated to dryness in vacuo. The crude product was then purified by crystallization using 100% hexane to yield 2.5 g (92%) of Compound 2.

C. Compound 3: (S)-3-(4-(4-Azidobutyloxy)phenyl-2-N-tert-butyloxycarbonyl-propionic acid benzyl ester as Illustrated in FIG. 21 Step ii.

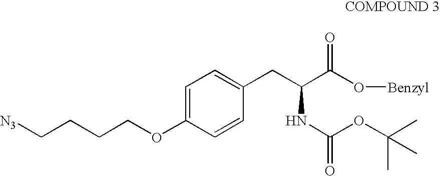

Compound 2 (2.5 g, 4.9 mmol) was stirred with sodium azide (1.6 9, 25 mmol) in dimethylformamide (DMF) (20 ml) at 25° C. for 12 hours. The solvent was then evaporated and the residue was treated with water (approx 10 ml) and extracted twice with ethyl acetate. The organic layers were combined, dried via magnesium sulfate and evaporated to yield 2.0 grams (90%) of Compound 3 as a colorless syrup (FAB-MS: 469 (M+H⁺).

D. Compound 4: (S)-3-(4-(4-Azidobutyloxy)phenyl-2-amino-propionic acid benzyl ester as Illustrated in FIG. 21 Step iii.

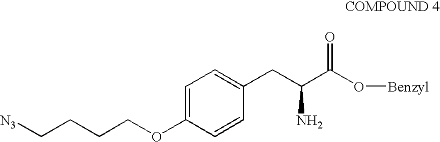

Compound 3 (2.0 g (4.4 mmol)) was dissolved in trifluoroacetic acid (TFA; 2 ml) and stirred for 3 hours at room temperature. Evaporation in vacuo yielded 1.6 grams (quantitative) of Compound 4 as a colorless syrup that was used without further purification for the next step. FAB-MS: 369 (M+H⁺).

E. Compound 5: (S)-3-(4-(4-Azidobutyloxy)phenyl-2-butylsulfonamido-propionic acid benzyl ester as Illustrated in FIG. 21 Step iv.

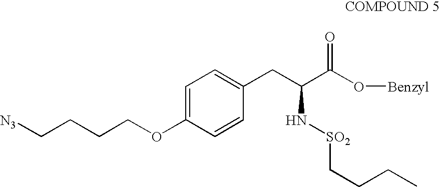

A mixture of Compound 4 (1.6 g; 4.3 mmol), butane sulfonic acid chloride (0.84 ml; 6.6 mmol) and triethyl amine (1.5 equivalents) were stirred in methylene chloride (20 ml) for 12 hours at room temperature. The reaction mixture was then evaporated and the residue was dissolved in ethylacetate, washed with dilute HCl, aqueous sodium bicarbonate and water. After evaporation to dryness the crude product was purified by flash chromatography (silica gel, toluene/ethylacetate 15:1) to yield 1.4 grams (67%) of Compound 5 as an amorphous solid.

F. Compound 6: (S)-3-(4-(4-Aminobutyloxy)phenyl-2-butylsulfonamido-propionic acid as Illustrated in FIG. 21 Step v.

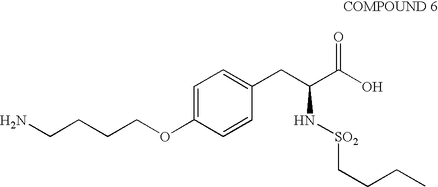

Compound 5 (1.3 g (2.6 mmol) was dissolved in 20 ml of ethyl acetate/methanol/water 5/3/1 and 0.2 ml trifluoroacetic acid (TFA) and hydrogenated under hydrogen (1 atmosphere; Parr Shaker apparatus) at 25° C. in the presence of 100 mg palladium (10% on charcoal). After 3 hours, the catalyst was filtered off and the solvent was evaporated to yield Compound 6 as an oily residue. After lyophilization from water 1.0 gram (quantitative) of Compound 6 was obtained as a white powder. FAB-MS: 373 (M⁺H⁺).

G. Compound 7: (S)-3-(4-(4-Guanidinobutyloxy)phenyl-2-butylsulfonamido-propionic acid as Illustrated in FIG. 21 Step vi.

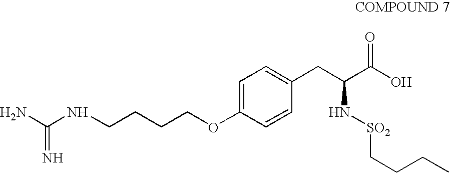

Compound 6 (200 mg; 0.5 mmol), 3, 5-dimethylpyrazol-1-carboxamidine nitrate (DPFN) (170 mg; 0.8 mmol; Aldrich Chemical Company) and triethylamine (0.15 ml, 1.0 mmol) in dimethylformamide (DMF; 5 ml) were heated at 60° C. for 12 hours. After cooling, the solvent was evaporated in vacuo, and the residue was purified by HPLC (Lichrocart RP-18, gradient acetonitrile/water +0.3% TFA 99:1 to 1:99) to yield 50 mg (25%) of Compound 7 as a white, amorphous powder, after lyophilization. FAB-MS: 415 (M⁺H⁺), m.p.: 70° C.

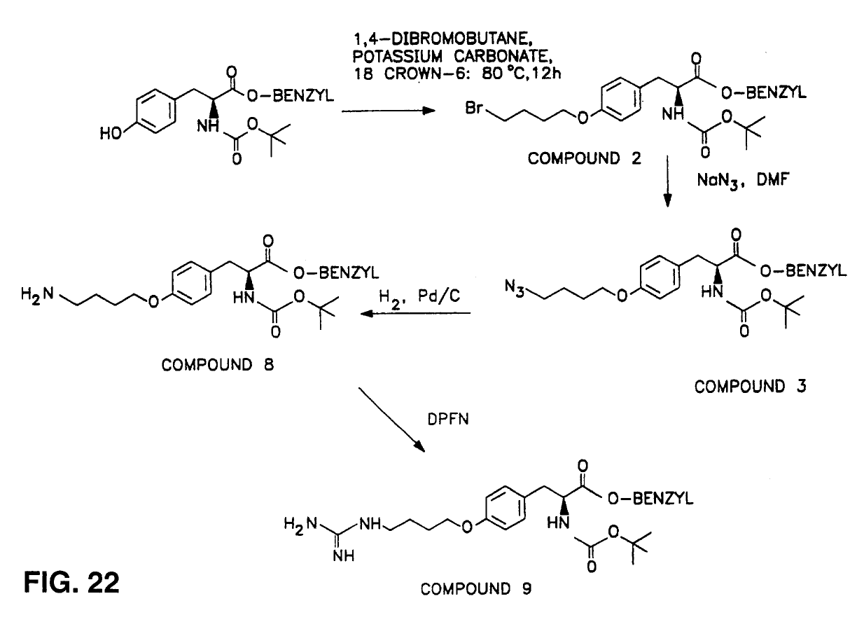

H. Compound 8: (S)-3-(4-(4-Aminobutyloxy)phenyl-2-N-tert.butyloxycarbonyl-propionic acid as Illustrated in FIG. 22 Step iii.

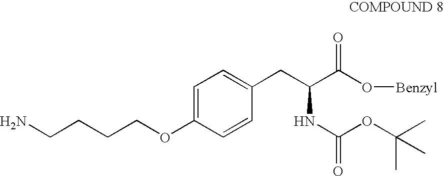

Compound 3 (0.5 9 (1.07 mmol) was dissolved in 10 ml of ethyl acetate/methanol/water 5/3/1 and 0.1 ml trifluoroacetic acid (TFA) and hydrogenated under hydrogen (1 atmosphere; Parr Shaker apparatus) at 25° C. in the presence of 30 mg palladium (10% on charcoal). After 3 hours, the catalyst was filtered off and the solvent was evaporated to yield Compound 8 as an oily residue. After lyophilization from water 370 milligram (quantitative) of Compound 8 was obtained as a white powder. FAB-MS: 353 (M⁺H⁺).

I. Compound 9: (S)-3-(4-(4-Guanidinobutyloxy)phenyl-2-N-tert.butyloxycarbonyl-propionic acid as Illustrated in FIG. 22 Step iv.

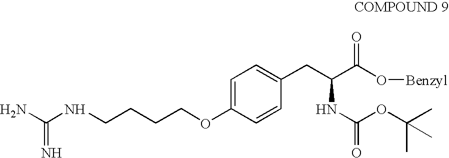

Compound 8 (200 mg; 0.5 mmol), 3, 5-dimethylpyrazol-1-carboxamidine nitrate (DPFN) (170 mg; 0.8 mmol; Aldrich Chemical Company) and triethylamine (0.15 ml, 1.0 mmol) in dimethylformamide (DMF; 5 ml) were heated at 60° C. for 12 hours. After cooling, the solvent was evaporated in vacuo, and the residue was purified by HPLC (Lichrocart RP-18, gradient acetonitrile/water +0.3% TFA 99:1 to 1:99) to yield 160 mg (90%) of Compound 9 as a white, amorphous powder, after lyophilization. FAB-MS: 395 (M⁺H⁺).

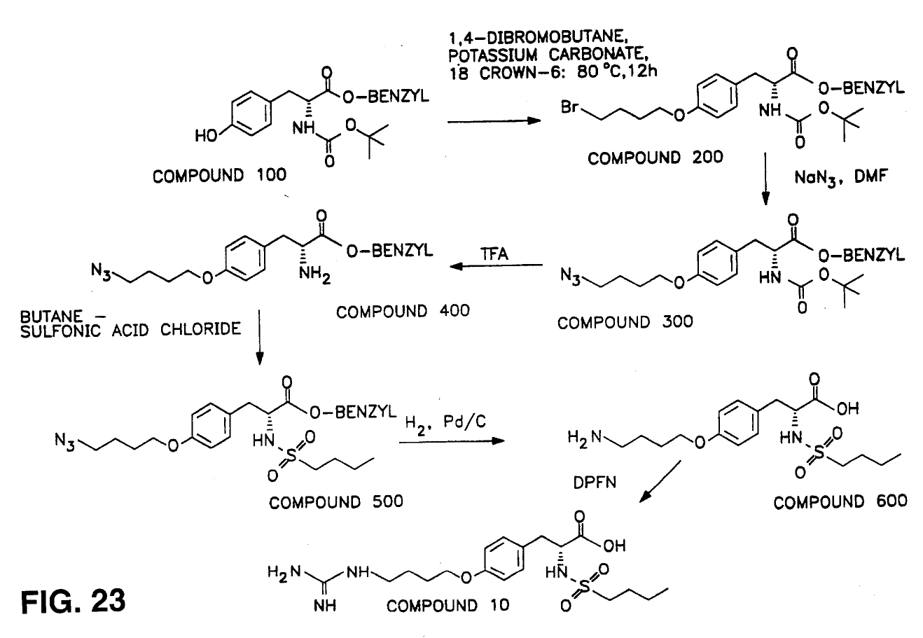

J. Compound 10: (R)-3-(4-(4-Guanidinobutyloxy)phenyl-2-butylsulfonamido-propionic acid as Illustrated in FIG. 23 Steps i-vi.

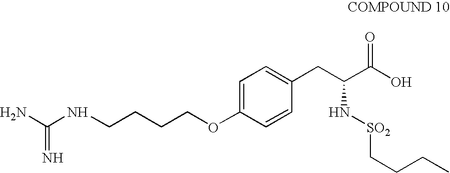

The identical reaction sequence to synthesize Compound 7 was used to prepare the D-tyrosine analog 10 of which 205 mg were obtained as a white amorphous material FAB-MS: 415 (M⁺H⁺) as follows using intermediate Compounds 100-600 to form Compound 10:

1) Compound 100: t-Boc-D-tyrosine benzyl ester as Illustrated in FIG. 23.

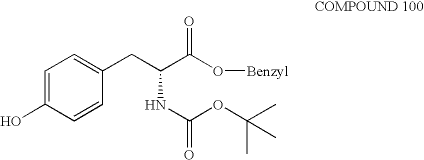

To a solution of N-(tert-butoxycarbonyl)D-tyrosine(t-Boc-L-tyrosine) (1.0 equivalents; Aldrich) in 0.10 M methylene chloride was added dicyclohexyl carbodiimide (DCCI) (1.5 equivalents) at 25° C. and allowed to stir for 1 hour. Next, 1.5 equivalents benzyl alcohol was added and the mixture was stirred for an additional 12 hours at 25° C. The reaction mixture was then diluted with ethyl acetate (0.10 M) and washed 2× with water, 1× with brine and dried over magnesium sulfate. The solvent was then removed in vacuo and the crude product was then purified by silica gel column chromatography.

2) Compound 200: (R)-3-(4-(4-Bromobutyloxy)phenyl-2-N-tert-butyloxycarbonyl-propionic acid benzyl ester as Illustrated in FIG. 23 step i.

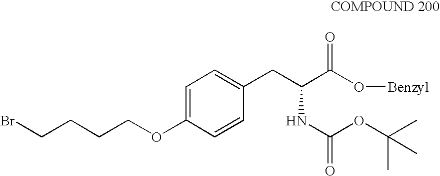

A mixture of t-Boc-D-tyrosine benzyl ester (2 grams, 5.38 mmol; synthesized as described above), 1, 4-dibromobutane (1.9 ml, 16.2 mmol; Aldrich), potassium carbonate (5 g) and 18-crown-6 (0.1 g; Aldrich), was heated at 80° C. for 12 hours. After cooling, the precipate was filtered off and the reaction mixture was evaporated to dryness in vacuo. The crude product was then purified by crystallization using 100% hexane to yield 2.5 g (92%) of Compound 200.

3) Compound 300: (R)-3-(4-(4-Azidobutyloxy)phenyl-2-N-tert-butyloxycarbonyl-propionic acid benzyl ester as Illustrated in FIG. 23 Step ii.

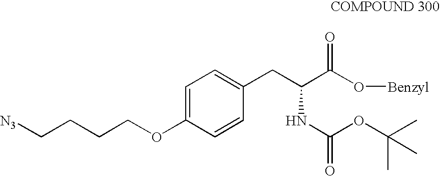

Compound 200 (2.5 9, 4.9 mmol) was stirred with sodium azide (1.6 g, 25 mmol) in dimethylformamide (DMF) (20 ml) at 25° C. for 12 hours. The solvent was then evaporated and the residue was treated with water (approx 10 ml) and extracted twice with ethyl acetate. The organic layers were combined, dried via magnesium sulfate and evaporated to yield 2.0 grams (90%) of Compound 300 as a colorless syrup (FAB-MS: 469 (M+H⁺).

4) Compound 400: (R)-3-(4-(4-Azidobutyloxy)phenyl-2-amino-propionic acid benzyl ester as Illustrated in FIG. 23 Step iii.

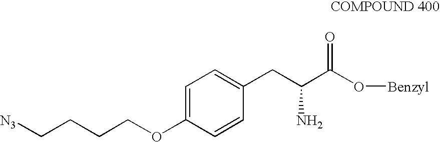

Compound 300 (2.0 g (4.4 mmol)) was dissolved in trifluoroacetic acid (TFA; 2 ml) and stirred for 3 hours at room temperature. Evaporation in vacuo yielded 1.6 grams (quantitative) of Compound 400 as a colorless syrup that was used without further purification for the next step. FAB-MS: 369 (M⁺H⁺).

5) Compound 500: (R)-3-(4-(4-Azidobutyloxy)phenyl-2-butylsulfonamido-propionic acid benzyl ester as Illustrated in FIG. 23 Step iv.

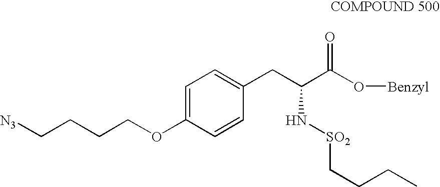

A mixture of Compound 400 (1.6 g; 4.3 mmol), butane sulfonic acid chloride (0.84 ml; 6.6 mmol) and triethyl amine (1.5 equivalents) were stirred in methylene chloride (20 ml) for 12 hours at room temperature. The reaction mixture was then evaporated and the residue was dissolved in ethylacetate, washed with dilute HCl, aqueous sodium bicarbonate and water. After evaporation to dryness the crude product was purified by flash chromatography (silica gel, toluene/ethyl acetate 15:1) to yield 1.4 grams (67%) of Compound 500 as an amorphous solid.

6) Compound 600: (R)-3-(4-(4-Aminobutyloxy)phenyl-2-butylsulfonamido-propionic acid as Illustrated in FIG. 23 Step v.

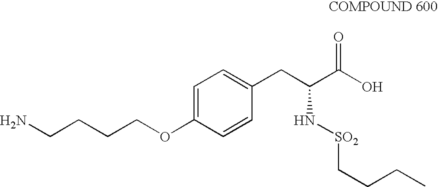

Compound 500 (1.3 9 (2.6 mmol) was dissolved in 20 ml of ethyl acetate/methanol/water 5/3/1 and 0.2 ml trifluoroacetic acid (TFA) and hydrogenated under hydrogen (1 atmosphere; Parr Shaker apparatus) at 25° C. in the presence of 100 mg palladium (10% on charcoal). After 3 hours, the catalyst was filtered off and the solvent was evaporated to yield Compound 600 as an oily residue. After lyophilization from water 1.0 gram (quantitative) of Compound 600 was obtained as a white powder. FAB-MS: 373 (M⁺H⁺).

7) Compound 10: (R)-3-(4-(4-Guanidinobutyloxy)phenyl-2-butylsulfonamido-propionic acid as Illustrated in FIG. 23 Step vi.

Compound 600 (200 mg; 0.5 mmol), 3, 5-dimethylpyrazol-1-carboxamidine nitrate (DPFN) (170 mg; 0.8 mmol; Aldrich Chemical Company) and triethylamine (0.15 ml, 1.0 mmol) in dimethylformamide (DMF; 5 ml) were heated at 60° C. for 12 hours. After cooling, the solvent was evaporated in vacuo, and the residue was purified by HPLC (Lichrocart RP-18, gradient acetonitrile/water +0.3% TFA 99:1 to 1:99) to yield 50 mg (25%) of Compound 10 as a white, amorphous powder, after lyophilization. FAB-MS: 415 (M⁺H⁺), m.p.: 70° C.

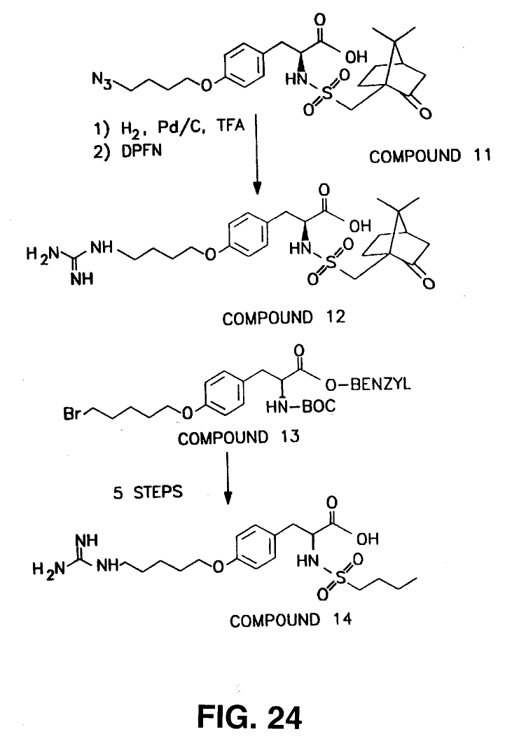

K. Compound 11: (S)-3-(4-(4-Azidobutyloxy)phenyl-2-(10-camphorsulfonamido)-propionic acid benzyl ester as Illustrated in FIG. 24.

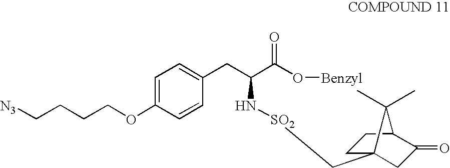

A mixture of compound 4 (1.0 g; 2.7 mmol), 10-camphorsulfonic acid chloride (6.6 mmol; Aldrich Chemical Company) and triethyl amine (1.5 equivalents) were stirred in methylene chloride (20 mL) for 12 hours at room temperature. The reaction mixture was then evaporated and the residue was dissolved in ethyl acetate, washed with dilute HCl, aqueous sodium bicarbonate and water. After evaporation to dryness the crude product was purified by flash chromatography (silica gel, toluene/ethyl acetate 15:1) to yield 1.4 grams (67%) of compound 11 as an amorphous solid.

L. Compound 12: (S)-3-(4-(4-Guanidinobutyloxy)phenyl-2-(10-camphorsulfonamido)-propionic acid as Illustrated in FIG. 24 Steps i-ii.

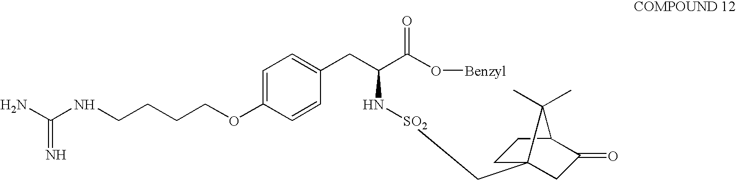

Compound 12 was obtained after hydrogenation and guanylation of Compound 11 according to the following conditions:

Step i: Compound 11 (1.3 g (2.6 mmol) was dissolved in 20 ml of ethyl acetate/methanol/water 5/3/1 and 0.2 ml trifluoroacetic acid (TFA) and hydrogenated under hydrogen (1 atmosphere; Parr Shaker apparatus) at 25° C. in the presence of 100 mg palladium (10% on charcoal). After 3 hours, the catalyst was filtered off and the solvent was evaporated to yield the intermediate amine as an oily residue. After lyophilization from water 1.0 gram (quantitative) of the intermediate amine was obtained as a white powder, which was carried on as follows:

Step ii: The above formed intermediate amine compound (200 mg; 0.5 mmol), 3, 5-dimethylpyrazol-1-carboxamidine nitrate (DPFN) (170 mg; 0.8 mmol; Aldrich Chemical Company) and triethylamine (0.15 ml, 1.0 mmol) in dimethylformamide (DMF; 5 ml) were heated at 60° C. for 12 hours. After cooling, the solvent was evaporated in vacuo, and the residue was purified by HPLC (Lichrocart RP-18, gradient acetonitrile/water +0.3% TFA 99:1 to 1:99) to yield 50 mg (25%) of Compound 12 as a white, amorphous powder, after lyophilization. FAB-MS: 509.6 (M⁺H⁺).

M. Compound 13: (S)-3-(4-(5-Bromopentyloxy)phenyl-2-N-tert.butyloxycarbonyl-propionic acid benzyl ester as Illustrated in FIG. 24.

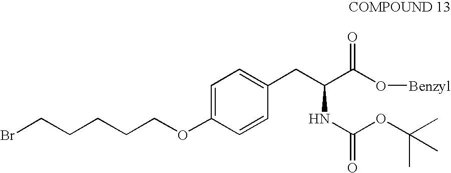

A mixture of t-Boc-L-tyrosine benzyl ester (4.5 grams, 12.1 mmol; Compound 1 synthesized as described above), 1, 5-dibromopentane (5 ml, 36.7 mmol; Aldrich), potassium carbonate (10 g) and 18-crown-6 (0.25 g; Aldrich), was heated at 80° C. for 12 hours. After cooling, the precipate was filtered off and the reaction mixture was evaporated to dryness in vacuo. The crude product was then purified by crystallization using 100% hexane to yield 5.35 g (85%) of Compound 13.

N. Compound 14: (S)-3-(4-(5-Guanidinopentyloxy)phenyl-2-butylsulfonamido-propionic acid as Illustrated in FIG. 24 Steps i-v.

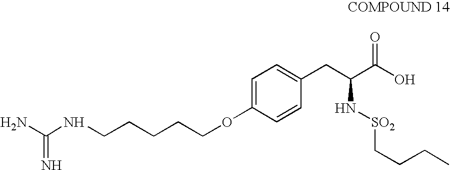

The 5 step reaction sequence of bromine-azide-exchange, Boc-cleavage, sulfonylation with butane sulfonic acid chloride, hydrogenation and quanylation with DPFN was carried out identically to the above procedures using intermediate Compounds 1-6 to form Compound 7 or the procedures using Compounds 100-600 to form Compound 10, as disclosed above. Compound 14 was obtained as a white powder FAB-MS: 429 (M⁺H⁺).

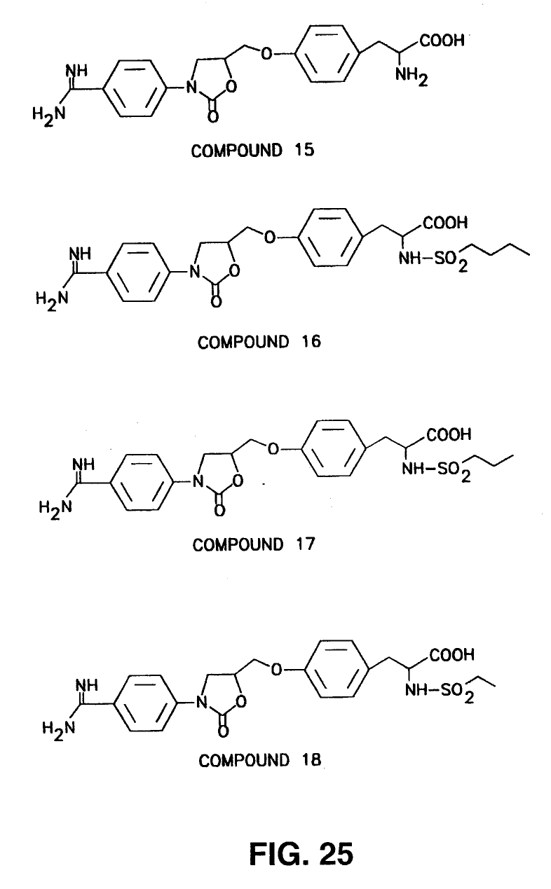

O. Compound 15: 3-(4-amidinophenyl)-5-(4-(2-carboxy-2-amino-ethyl)phenoxy)methyl-2-oxazolidinone, dihydrochloride as Shown in FIG. 25.

1) Synthesis of Starting Material 2-N-BOC-amino-3-(4-hydroxy-phenyl)propionate for Compound 15.

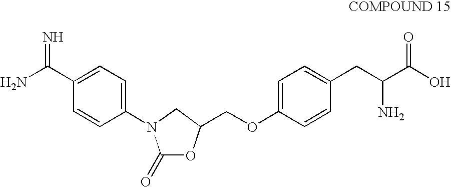

The starting material 2-N-BOC-amino-3-(4-hydroxy-phenyl)propionate was obtained via esterification of (D or L), N-(tert-butoxycarbonyl)-L(D)-tyrosine (t-Boc-L(D)-tyrosine) (1.0 equivalents; Sigma) in 0.10 M methanol and dilute 1% HCl. The reaction mixture was stirred at 25° C. for 12 hours and then neutralized via potassium carbonate and then diluted with ethyl acetate (0.10 M) and washed 2× with water, 1× with brine and dried over magnesium sulfate. The solvent was then removed in vacuo and the crude product was then purified by silica gel column chromatography to obtain 2-N-BOC-amino-3-(4-hydroxy-phenyl)propionate.

2) Synthesis of Starting Material 3-p-N-BOC-amidino-phenyl-5-methanesulfonyloxy-methyl-2-oxazolidinone for Compound 15. 3-Step Procedure as Follows:

p-amino-benzonitrile (1.0 equivalents; Aldrich) in methylene chloride (0.10 M) was stirred with 2, 3-epoxypropanol (1.0 equivalents; Aldrich) for 12 hours at 25° C. The solvent was next removed in vacuo and the crude 4-(2, 3-dihydroxypropylamino)benzonitrile was carried onto the next step as follows:

4-(2, 3-dihydroxypropylamino)benzonitrile (1.0 equivalents; as described above), in dimethylformamide (0.10 M), at 25° C., was stirred with diethyl carbonate (1.1 equivalents; Aldrich) and potassium tert-butylate (1.1 equivalents; Aldrich) at 110° C. for 6 hours. Next, the reaction mixture was diluted with ethyl acetate (0.10 M) and washed 2× with water, 1× with brine and dried over magnesium sulfate. The solvent was then removed in vacuo and the crude product was then purified by silica gel column chromatography to obtain 3-(4-cyanophenyl)-5-hydroxymethyl-2-oxazolidine and carried onto the next step as follows:

3-(4-cyanophenyl)-5-hydroxymethyl-2-oxazolidine (1.0 equivalents; as described above), in methylene chloride (0.10 M) at 25° C. was stirred with 1.1 equivalents hydrogen sulfide, 1.1 equivalents methyl iodide, and 1.1 equivalents ammonium acetate. The reaction mixture was stirred for 6 hours and then diluted with ethyl acetate (0.10 M) and washed 2× with water, 1× with brine and dried over magnesium sulfate. The solvent was then removed in vacuo and the crude product was then purified by silica gel column chromatography to obtain the amidine which was carried onto the next step as follows:

1.0 equivalents of the amidine, synthesized as described above, was protected with 1.1 equivalents of BOC-ON (2-(BOC-oxyimino)-2-phenylacetonitrile; Aldrich) in methylene chloride (0.10 M) at 25° C. and stirred for 6 hours. Next, the reaction mixture was diluted with ethyl acetate (0.10 M) and washed 2× with water, 1× with brine and dried over magnesium sulfate. The solvent was then removed in vacuo and the crude product was then esterified in 0.10 M methylene chloride and 1.1 equivalents methanesulfonyl chloride. The reaction mixture was stirred at 0° C. for 6 hours and then quenched with water (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain 3-p-N-BOC-amidino-phenyl-5-methanesulfonyloxy-methyl-2-oxazolidinone.

3) Coupling of Intermediates 2-N-BOC-amino-3-(4-hydroxy-phenyl)propionate with 3-p-N-BOC-amidino-phenyl-5-methanesulfonyloxy-methyl-2-oxazolidinone to Form Protected Form of Compound 15, 3-(4-BOC-amidinophenyl)-5-(4-(2-methoxy-carbonyl-2N-BOC-aminoethyl)phenyoxylmethyl-2-oxazolidinone.

A mixture of 1.9 grams 2-N-BOC-amino-3-(4-hydroxy-phenyl)propionate (as described above), 20 ml dimethylformamide (DMF) and NaH (1.0 equivalent), was stirred for 30 minutes at room temperature. After stirring, 1.8 grams 3-p-N-BOC-amidino-phenyl-5-methanesulfonyloxy-methyl-2-oxazolidinone (as described above) in 10 ml dimethylformamide (DMF) was added and stirred again for 15 minutes at room temperature. The reaction mixture was then diluted with ethyl acetate (0.10 M) and washed 2× with water, 1× with brine and dried over magnesium sulfate. The solvent was then removed in vacuo and the crude product was then purified by silica gel column chromatography to obtain protected form of Compound 15, 3-(4-BOC-amidinophenyl)-5-(4-(2-methoxy-carbonyl-2N-BOC-aminoethyl)phenylmethyl-2-oxazolidinone which was carried onto the next step.

4) Deprotection of Protected Form of Compound 15 to Form Compound 15: 3-(4-amidinophenyl)-5-(4-(2-carboxy-2-amino-ethyl)phenoxy)methyl-2-oxazolidinone, dihydrochloride, FIG. 25.

Treatment of the protected form of Compound 15, 3-(4-BOC-amidinophenyl)-5-(4-(2-methoxy-carbonyl-2N-BOC-aminoethyl)phenyoxylmethyl-2-oxazolidinone (1.0 equivalents; synthesized as described above), with 4 ml 2N NaOH for 4 hours at room temperature. The mixture was then followed with 40 ml 2N HCl-solution in dioxane added dropwise at 0° C. to 25° C. for 3 hours. The reaction mixture was then quenched with sodium bicarbonate (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain Compound 15: 3-(4-amidinophenyl)-5-(4-(2-carboxy-2-amino-ethyl)phenoxy)methyl-2-oxazolidinone, dihydrochloride; m.p. 165° C.(d).

P. Compound 16: 3-(4-amidinophenyl)-5-(4-(2-carboxy-2-N-butylsulfonylaminoethyl)phenoxy)methyl-2-oxazolidinone as Shown in FIG. 25.

1) Synthesis of Starting Material 2-N-butylsulfonylamino-3-(4-hydroxy-phenyl)propionate for Compound 16.

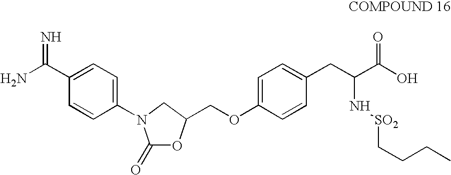

The starting material 2-N-butylsulfonylamino-3-(4-hydroxy-phenyl)propionate was obtained via esterification of ((D or L) tyrosine) (1.0 equivalents; Sigma) in 0.10 M methanol and dilute 1% HCl. The reaction mixture was stirred at 25° C. for 12 hours and then neutralized via potassium carbonate and then diluted with ethyl acetate (0.10 M) and washed 2× with water, 1× with brine and dried over magnesium sulfate. The solvent was then removed in vacuo and the crude product was then carried on as follows:

A mixture of the above compound (4.3 mmol), butane sulfonic acid chloride (6.6 mmol) and triethyl amine (1.5 equivalents) were stirred in methylene chloride (20 ml) for 12 hours at room temperature. The reaction mixture was then evaporated and the residue was dissolved in ethyl acetate, washed with dilute HCl, aqueous sodium bicarbonate and water. After evaporation to dryness the crude product was purified by flash chromatography (silica gel, toluene/ethyl acetate 15:1) to yield the title compound.

2) Synthesis of Starting Material 3-p-N-BOC-amidino-phenyl-5-methanesulfonyloxy-methyl-2-oxazolidinone for Compound 16. 3-Step Procedure as Follows:

p-amino-benzonitrile (1.0 equivalents; Aldrich) in methylene chloride (0.10 M) was stirred with 2, 3-epoxypropanol (1.0 equivalents; Aldrich) for 12 hours at 25° C. The solvent was next removed in vacuo and the crude 4-(2, 3-dihydroxypropylamino)benzonitrile was carried onto the next step as follows:

4-(2, 3-dihydroxypropylamino)benzonitrile (1.0 equivalents; as described above), in dimethylformamide (0.10 M), at 25° C., was stirred with diethyl carbonate (1.1 equivalents; Aldrich) and potassium tert-butylate (1.1 equivalents; Aldrich) at 110° C. for 6 hours. Next, the reaction mixture was diluted with ethyl acetate (0.10 M) and washed 2× with water, 1× with brine and dried over magnesium sulfate. The solvent was then removed in vacuo and the crude product was then purified by silica gel column chromatography to obtain 3-(4-cyanophenyl)-5-hydroxymethyl-2-oxazolidine and carried onto the next step as follows:

3-(4-cyanophenyl)-5-hydroxymethyl-2-oxazolidine (1.0 equivalents; as described above), in methylene chloride (0.10 M) at 25° C. was stirred with 1.1 equivalents hydrogen sulfide, 1.1 equivalents methyl iodide, and 1.1 equivalents ammonium acetate. The reaction mixture was stirred for 6 hours and then diluted with ethyl acetate (0.10 M) and washed 2× with water, 1× with brine and dried over magnesium sulfate. The solvent was then removed in vacuo and the crude product was then purified by silica gel column chromatography to obtain the amidine which was carried onto the next step as follows:

1.0 equivalents of the amidine, synthesized as described above, was protected with 1.1 equivalents of BOC-ON (2-(BOC-oxyimino)-2-phenylacetonitrile; Aldrich) in methylene chloride (0.10 M) at 25° C. and stirred for 6 hours. Next, the reaction mixture was diluted with ethyl acetate (0.10 M) and washed 2× with water, 1× with brine and dried over magnesium sulfate. The solvent was then removed in vacuo and the crude product was then esterified in 0.10 M methylene chloride and 1.1 equivalents methanesulfonyl chloride. The reaction mixture was stirred at 0° C. for 6 hours and then quenched with water (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain 3-p-N-BOC-amidino-phenyl-5-methanesulfonyloxy-methyl-2-oxazolidinone.

3) Coupling of Intermediates 2-N-butylsulfonylamino-3-(4-hydroxy-phenyl)propionate with 3-p-N-BOC-amidino-phenyl-5-methanesulfonyloxy-methyl-2-oxazolidinone to Form Protected Form of Compound 16, 3-(4-BOC-amidinophenyl)-5-(4-(2-methoxy-carbonyl-2-N-butylsulfonylaminoethyl)phenyoxylmethyl-2-oxazolidinone.

A mixture of 1.9 grams 2-N-butylsulfonylamino-3-(4-hydroxy-phenyl)propionate (as described above), 20 ml dimethylformamide (DMF) and NaH (1.0 equivalent), was stirred for 30 minutes at room temperature. After stirring, 1.8 grams 3-p-N-BOC-amidino-phenyl-5-methanesulfonyloxy-methyl-2-oxazolidinone (as described above) in 10 ml dimethylformamide (DMF) was added and stirred again for 15 minutes at room temperature. The reaction mixture was then diluted with ethyl acetate (0.10 M) and washed 2× with water, 1× with brine and dried over magnesium sulfate. The solvent was then removed in vacuo and the crude product was then purified by silica gel column chromatography to obtain protected form of Compound 16, 3-(4-BOC-amidinophenyl)-5-(4-(2-methoxy-carbonyl-2-N-butylsulfonylaminoethyl)-phenyoxylmethyl-2-oxazolidinone which was carried onto the next step.

4) Deprotection of Protected Form of Compound 16 to Form Compound 16: 3-(4-amidinophenyl)-5-(4-(2-carboxy-2-N-butylsulfonylaminoethyl)phenoxy)methyl-2-oxazolidinone, FIG. 25.

Treatment of the protected form of Compound 16, 3-(4-BOC-amidinophenyl)-5-(4-(2-methoxy-carbonyl-2-N-butylsulfonylaminoethyl)phenyoxylmethyl-2-oxazolidinone (1.0 equivalents; synthesized as described above), with 4 ml 2N NaOH for 4 hours at room temperature. The mixture was then followed with 40 ml 2N HCl-solution in dioxane added dropwise at 0° C. to 25° C. for 3 hours. The reaction mixture was then quenched with sodium bicarbonate (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain Compound 16: 3-(4-amidinophenyl)-5(4-(2-carboxy-2-N-butylsulfonylaminoethyl)phenoxy)methyl-2-oxazolidinone; m.p. 236-237° C.

Q. Compound 17: 3-(4-amidinophenyl)-5-(4-(2-carboxy-2-N-propyl-sulfonylaminoethyl)phenoxy)methyl-2-oxazolidinone as Shown in FIG. 25.

1) Synthesis of Starting Material 2-N-propyl-sulfonylamino-3-(4-hydroxy-phenyl)propionate for Compound 17.

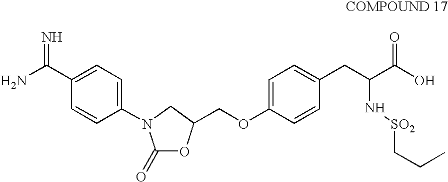

The starting material 2-N-propyl-sulfonylamino-3-(4-hydroxy-phenyl)propionate was obtained via esterification of ((D or L) tyrosine) (1.0 equivalents; Sigma) in 0.10 M methanol and dilute 1% HCl. The reaction mixture was stirred at 25° C. for 12 hours and then neutralized via potassium carbonate and then diluted with ethyl acetate (0.10 M) and washed 2× with water, 1× brine and dried over magnesium sulfate. The solvent was then removed in vacuo and the crude product was then carried on as follows:

A mixture of the above compound (4.3 mmol), propyl sulfonic acid chloride (6.6 mmol; Aldrich) and triethyl amine (1.5 equivalents) were stirred in methylene chloride (20 ml) for 12 hours at room temperature. The reaction mixture was then evaporated and the residue was dissolved in ethyl acetate, washed with dilute HCl, aqueous sodium bicarbonate and water. After evaporation to dryness the crude product was purified by flash chromatography (silica gel, toluene/ethyl acetate 15:1) to yield the title compound.

2) Synthesis of starting material 3-p-N-BOC-amidino-phenyl-5-methanesulfonyloxy-methyl-2-oxazolidinone for Compound 17. 3-Step Procedure as Follows:

p-amino-benzonitrile (1.0 equivalents; Aldrich) in methylene chloride (0.10 M) was stirred with 2, 3-epoxypropanol (1.0 equivalents; Aldrich) for 12 hours at 25° C. The solvent was next removed in vacuo and the crude 4-(2, 3-dihydroxypropylamino)benzonitrile was carried onto the next step as follows:

4-(2, 3-dihydroxypropylamino)benzonitrile (1.0 equivalents; as described above), in dimethylformamide (0.10 M), at 25° C., was stirred with diethyl carbonate (1.1 equivalents; Aldrich) and potassium tert-butylate (1.1 equivalents; Aldrich) at 110° C. for 6 hours. Next, the reaction mixture was diluted with ethyl acetate (0.10 M) and washed 2× with water, 1× with brine and dried over magnesium sulfate. The solvent was then removed in vacuo and the crude product was then purified by silica gel column chromatography to obtain 3-(4-cyanophenyl)-5-hydroxymethyl-2-oxazolidine and carried onto the next step as follows:

3-(4-cyanophenyl)-5-hydroxymethyl-2-oxazolidine (1.0 equivalents; as described above), in methylene chloride (0.10 M) at 25° C. was stirred with 1.1 equivalents hydrogen sulfide, 1.1 equivalents methyl iodide, and 1.1 equivalents ammonium acetate. The reaction mixture was stirred for 6 hours and then diluted with ethyl acetate (0.10 M) and washed 2× with water, 1× with brine and dried over magnesium sulfate. The solvent was then removed in vacuo and the crude product was then purified by silica gel column chromatography to obtain the amidine which was carried onto the next step as follows:

1.0 equivalents of the amidine, synthesized as described above, was protected with 1.1 equivalents of BOC-ON (2-(BOC-oxyimino)-2-phenylacetonitrile; Aldrich) in methylene chloride (0.10 M) at 25° C. and stirred for 6 hours. Next, the reaction mixture was diluted with ethyl acetate (0.10 M) and washed 2× with water, 1× with brine and dried over magnesium sulfate. The solvent was then removed in vacuo and the crude product was then esterified in 0.10 M methylene chloride and 1.1 equivalents methanesulfonyl chloride. The reaction mixture was stirred at 0° C. for 6 hours and then quenched with water (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain 3-p-N-BOC-amidino-phenyl-5-methanesulfonyloxy-methyl-2-oxazolidinone.

3) Coupling of intermediates 2-N-propyl-sulfonylamino-3-(4-hydroxy-phenyl)propionate with 3-p-N-BOC-amidino-phenyl-5-methanesulfonyloxy-methyl-2-oxazolidinone to Form Protected Form of Compound 17, 3-(4-BOC-amidinophenyl)-5-(4-(2-methoxy-carbonyl-2-N-propyl-sulfonylaminoethyl)-phenyoxylmethyl-2-oxazolidinone.

A mixture of 1.9 grams 2-N-propyl-sulfonylamino-3-(4-hydroxy-phenyl)propionate (as described above), 20 ml dimethylformamide (DMF) and NaH (1.0 equivalent), was stirred for 30 minutes at room temperature. After stirring, 1.8 grams 3-p-N-BOC-amidino-phenyl-5-methanesulfonyloxy-methyl-2-oxazolidinone (as described above) in 10 ml dimethylformamide (DMF) was added and stirred again for 15 minutes at room temperature. The reaction mixture was then diluted with ethyl acetate (0.10 M) and washed 2× with water, 1× with brine and dried over magnesium sulfate. The solvent was then removed in vacuo and the crude product was then purified by silica gel column chromatography to obtain protected form of Compound 17, 3-(4-BOC-amidinophenyl)-5-(4-(2-methoxy-carbonyl-2-N-propyl-sulfonylaminoethyl)-phenyoxylmethyl-2-oxazolidinone which was carried onto the next step.

4) Deprotection of Protected Form of Compound 17 to Form Compound 17: 3-(4-amidinophenyl)-5-(4-(2-carboxy-2-N-propylsulfonylaminoethyl)phenoxy)methyl-2-oxazolidinone, FIG. 25.

Treatment of the protected form of Compound 17, 3-(4-BOC-amidinophenyl)-5-(4-(2-methoxy-carbonyl-2-N-propylsulfonylaminoethyl)phenyoxylmethyl-2-oxazolidinone (1.0 equivalents; synthesized as described above), with 4 ml 2N NaOH for 4 hours at room temperature. The mixture was then followed with 40 ml 2N HCl-solution in dioxane added dropwise at 0° C. to 25° C. for 3 hours. The reaction mixture was then quenched with sodium bicarbonate (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain Compound 17: 3-(4-amidinophenyl)-5-(4-(2-carboxy-2-N-propylsulfonylaminoethyl)phenoxy)methyl-2-oxazolidinone; m.p. 200° C. (d).

R. Compound 18: 3-(4-amidinophenyl)-5-(4-(2-carboxy-2-N-ethyl-sulfonylaminoethyl) phenoxy)methyl-2-oxazolidinone as Shown in FIG. 25.

1) Synthesis of Starting Material 2-N-ethyl-sulfonylamino-3-(4-hydroxy-phenyl)propionate for Compound 18.

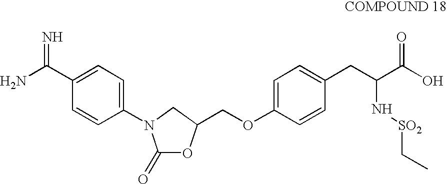

The starting material 2-N-ethyl-sulfonylamino-3-(4-hydroxy-phenyl)propionate was obtained via esterification of ((D or L) tyrosine) (1.0 equivalents; Sigma) in 0.10 M methanol and dilute 1% HCl. The reaction mixture was stirred at 25° C. for 12 hours and then neutralized via potassium carbonate and then diluted with ethyl acetate (0.10 M) and washed 2× with water, 1× with brine and dried over magnesium sulfate. The solvent was then removed in vacuo and the crude product was then carried on as follows:

A mixture of the above compound (4.3 mmol), ethyl sulfonic acid chloride (6.6 mmol; Aldrich) and triethyl amine (1.5 equivalents) were stirred in methylene chloride (20 ml) for 12 hours at room temperature. The reaction mixture was then evaporated and the residue was dissolved in ethyl acetate, washed with dilute HCl, aqueous sodium bicarbonate and water. After evaporation to dryness the crude product was purified by flash chromatography (silica gel, toluene/ ethyl acetate 15:1) to yield the title compound.

2) Synthesis of Starting Material 3-p-N-BOC-amidino-phenyl-5-methanesulfonyloxy-methyl-2-oxazolidinone for Compound 18. 3-Step Procedure as Follows:

p-amino-benzonitrile (1.0 equivalents; Aldrich) in methylene chloride (0.10 M) was stirred with 2, 3-epoxypropanol (1.0 equivalents; Aldrich) for 12 hours at 25° C. The solvent was next removed in vacuo and the crude 4-(2, 3-dihydroxypropylamino)benzonitrile was carried onto the next step as follows:

4-(2, 3-dihydroxypropylamino)benzonitrile (1.0 equivalents; as described above), in dimethylformamide (0.10 M), at 25° C., was stirred with diethyl carbonate (1.1 equivalents; Aldrich) and potassium tert-butylate (1.1 equivalents; Aldrich) at 110° C. for 6 hours. Next, the reaction mixture was diluted with ethyl acetate (0.10 M) and washed 2× with water, 1× with brine and dried over magnesium sulfate. The solvent was then removed in vacuo and the crude product was then purified by silica gel column chromatography to obtain 3-(4-cyanophenyl)-5-hydroxymethyl-2-oxazolidine and carried onto the next step as follows:

3-(4-cyanophenyl)-5-hydroxymethyl-2-oxazolidine (1.0 equivalents; as described above), in methylene chloride (0.10 M) at 25° C. was stirred with 1.1 equivalents hydrogen sulfide, 1.1 equivalents methyl iodide, and 1.1 equivalents ammonium acetate. The reaction mixture was stirred for 6 hours and then diluted with ethyl acetate (0.10 M) and washed 2× with water, 1× with brine and dried over magnesium sulfate. The solvent was then removed in vacuo and the crude product was then purified by silica gel column chromatography to obtain the amidine which was carried onto the next step as follows:

1.0 equivalents of the amidine, synthesized as described above, was protected with 1.1 equivalents of BOC-ON (2-(BOC-oxyimino)-2-phenylacetonitrile; Aldrich) in methylene chloride (0.10 M) at 25° C. and stirred for 6 hours. Next, the reaction mixture was diluted with ethyl acetate (0.10 M) and washed 2× with water, 1× with brine and dried over magnesium sulfate. The solvent was then removed in vacuo and the crude product was then esterified in 0.10 M methylene chloride and 1.1 equivalents methanesulfonyl chloride. The reaction mixture was stirred at 0° C. for 6 hours and then quenched with water (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain 3-p-N-BOC-amidino-phenyl-5-methanesulfonyloxy-methyl-2-oxazolidinone.

3) Coupling of Intermediates 2-N-ethyl-sulfonylamino-3-(4-hydroxy-phenyl)propionate with 3-p-N-BOC-amidino-phenyl-5-methanesulfonyloxy-methyl-2-oxazolidinone to Form Protected Form of Compound 18, 3-(4-BOC-amidinophenyl)-5-(4-(2-methoxy-carbonyl-2-N-ethyl-sulfonylaminoethyl)-phenyoxylmethyl-2-oxazolidinone.

A mixture of 1.9 grams 2-N-ethyl-sulfonylamino-3-(4-hydroxy-phenyl)propionate (as described above), 20 ml dimethylformamide (DMF) and NaH (1.0 equivalent), was stirred for 30 minutes at room temperature. After stirring, 1.8 grams 3-p-N-BOC-amidino-phenyl-5-methanesulfonyloxy-methyl-2-oxazolidinone (as described above) in 10 ml dimethylformamide (DMF) was added and stirred again for 15 minutes at room temperature. The reaction mixture was then diluted with ethyl acetate (0.10 M) and washed 2× with water, 1× with brine and dried over magnesium sulfate. The solvent was then removed in vacuo and the crude product was then purified by silica gel column chromatography to obtain protected form of Compound 18, 3-(4-BOC-amidinophenyl)-5-(4-(2-methoxy-carbonyl-2-N-ethyl-sulfonylaminoethyl)-phenyoxylmethyl-2-oxazolidinone which was carried onto the next step.

4) Deprotection of Protected Form of Compound 18 to form Compound 18: 3-(4-amidinophenyl)-5-(4-(2-carboxy-2-N-ethylsulfonylaminoethyl)phenoxy)methyl-2-oxazolidinone, FIG. 25.

Treatment of the protected form of Compound 18, 3-(4-BOC-amidinophenyl)-5-(4-(2-methoxy-carbonyl-2-N-ethylsulfonylaminoethyl)phenyoxylmethyl-2-oxazolidinone (1.0 equivalents; synthesized as described above), with 4 ml 2N NaOH for 4 hours at room temperature. The mixture was then followed with 40 ml 2N HCl-solution in dioxane added dropwise at 0° C. to 25° C. for 3 hours. The reaction mixture was then quenched with sodium bicarbonate (5 equivalents) and then diluted with ethyl acetate (0.10 M) and washed 2× with water, 1× with brine and dried over magnesium sulfate. The solvent was then removed in vacuo and the crude product was then purified by silica gel column chromatography to obtain Compound 18: 3-(4-amidinophenyl)-5-(4-(2-carboxy-2-N-ethylsulfonylaminoethyl)phenoxy)methyl-2-oxazolidinone; m.p. 212° C. (d).

### EXAMPLE 12

Inhibition of Growth Factor-Induced Angiogenesis as Measured in the CAM Assay with by Intravenous Application of αᵥβ₃ Ligand Organic Mimetics.

The effect on growth factor-induced angiogenesis with organic peptidomimetics of an αᵥβ₃ ligand intravenously injected into the CAM preparation was also evaluated for use in this invention.

The 10 day old CAM preparation was used as previously described in Example 5A. Twenty-four hours after bFGF-induced angiogenesis was initiated, the organic peptidomimetics referred to as compounds 16 (81218), 17 (87292) and 18 (87293) were separately intravenously injected into the CAM preparation in a volume of 100 μl at a concentration of 1 mg/ml (100 μg/embryo) in 20% tetraglycol-PBS at pH 7.0. In parallel assays, compounds 7 (96112), 9 (99799), 10 (96229), 12 (112854) and 14 (96113) were similarly evaluated. The effects of the organic mimetics were analyzed 48 hours later where quantification was performed by counting the number of blood vessel branch points in the area of the filter disc in a double blind approach.

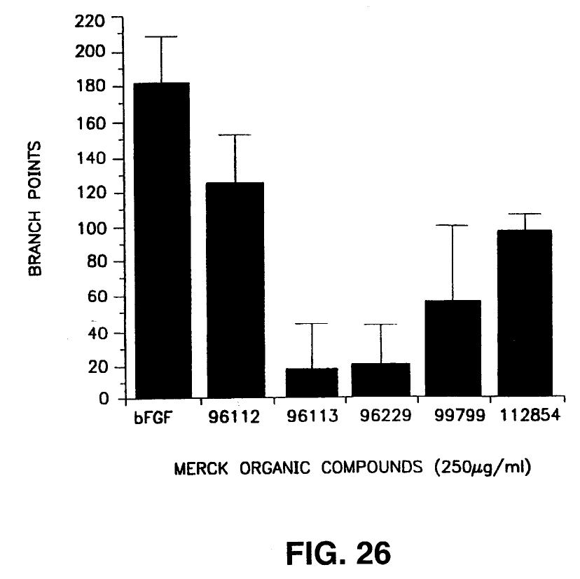

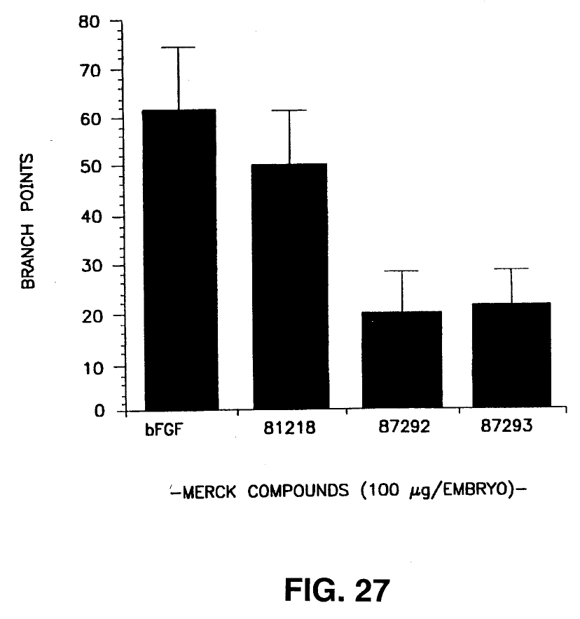

The results are respectively shown in FIGS. 26 and 27. In FIG. 26, compounds 14 (96113), 10 (96229), 9 (99799) and 12 (112854), in decreasing order of inhibition, were effective at reducing the number of branch points of new blood vessels compared to control bFGF induction and compared to compound 7 (96112). In FIG. 27, compounds 17 (87292) and 18 (87293) exhibited anti-angiogenic properties as compared to untreated bFGF control and treatment with compound 16 (81218).

In a third assay, organic compounds 7 (96112), 10 (96229) and 14 (96113) were assessed as inhibitors of bFGF-induced angiogenesis along with peptides 69601 and 66203. For this assay, 250 μg/ml of organic compounds were administered 18 hours after bFGF treatment as described in Example 5B. The results are shown in FIG. 12 where as above, compounds 14 (96113) and 10 (96229) almost completely inhibited the formation of new blood vessels induced by bFGF.

Compounds I(a) and I(g) through I(r), as shown in FIGS. 28-31, were also evaluated in this chick CAM bFGF-induced angiogenesis inhibition assay. The results are tabulated in Table 4, below. Levels of angiogenesis inhibition in the range of about 40% to greater than about 95% were observed.

TABLE 4

| Compound | Dosage (μg/egg) | % Inhibition |
|---|---|---|
| I(l) | 10 | >95 |
| I(n) | 10 | >95 |
| I(m) | 30 | >95 |
| I(p) | 1 | >95 |
| I(r) | 10 | 50 |
| I(q) | 10 | 70 |
| I(a) | 1 | 40 |
| I(o) | 1 | 75 |
| I(j) | 30 | >95 |
| I(k) | 30 | 85 |
| I(h) | 1 | >95 |
| I(i) | 10 | 75 |
| I(g) | 10 | 85 |

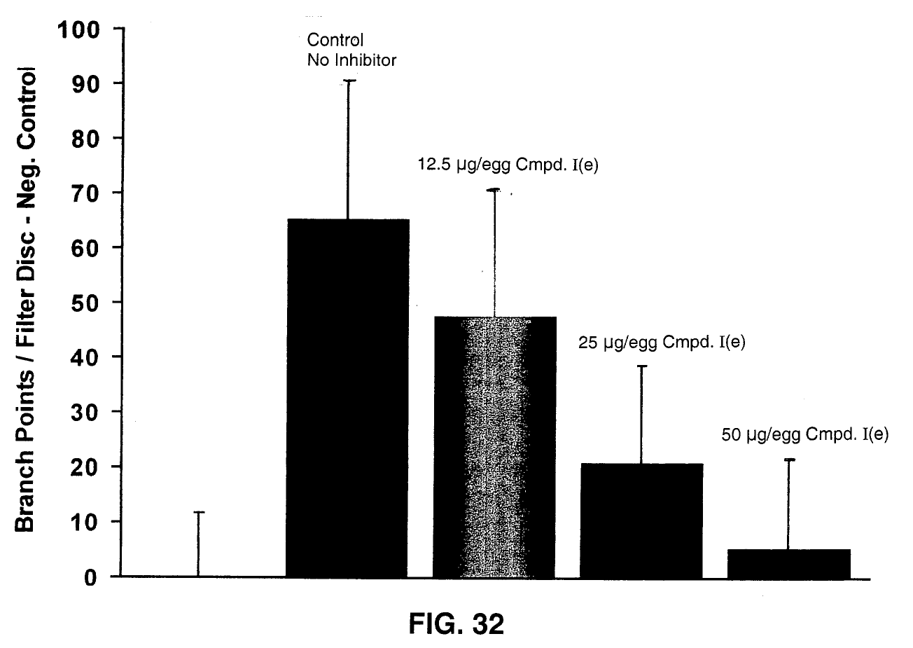

FIG. 32 graphically illustrates the reduction in vascular branch points observed in CAMs treated with Compound I(e) at three dosage levels ranging from about 12.5 μg/egg to about 50 μg/egg. The control CAM (no inhibitor) exhibited about 65 branch points, whereas CAMS treated with Compound I(e) exhibited an average of less than about 50 branch at a Compound I(e) dosage level of about 12.5 μg/egg, about 20 branch points at a dosage of about 25 μg/egg, and about 5 branch points at a dosage of about 50 μg/egg.

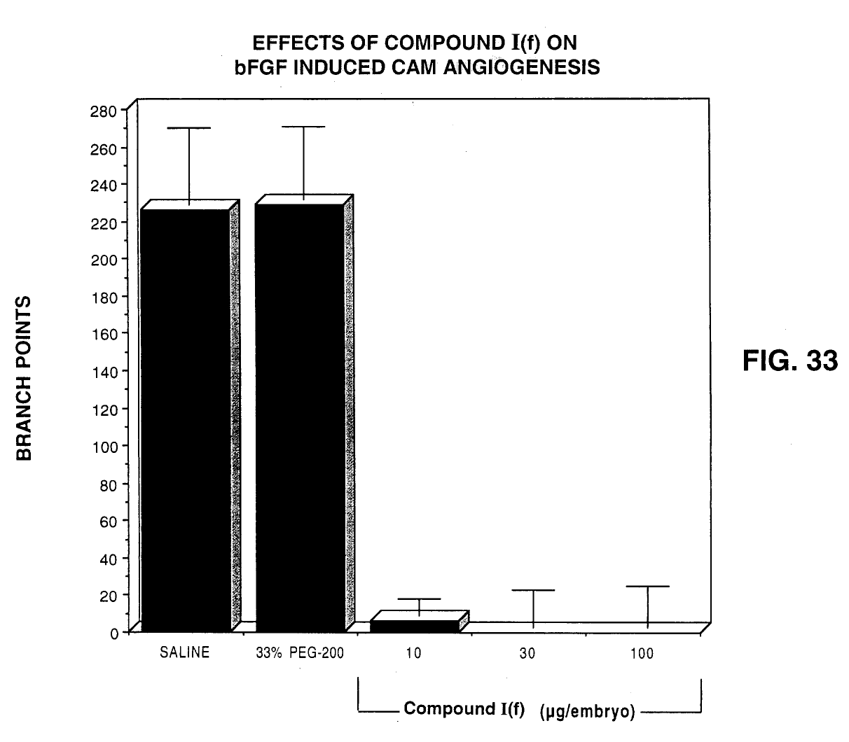

In similar parallel experiments in bFGF-induced angiogenesis, compound I(f) was shown to be effective at inhibiting angiogenesis at 10, 30 and 100 μg/embryo concentrations. For these experiments, 30 μl of a 2.5 μg/ml solution of bFGF was used to induce angiogenesis. Eggs were incubated for about 18 hours at 97° F. prior to intravenous application of Compound I(f) that was previously dissolved in polyethylene glycol (PEG-200) to result in a 4.4 mg/ml solution from which the final amounts of the compound were administered. In comparison to controls of saline and 33% PEG-200 alone, all three amounts of Compound I(f) significantly inhibited bFGF-induced angiogenesis as shown in FIG. 33.

### EXAMPLE 13

Tumor Growth Inhibition in Athymic Nude Mice: Inhibition of M21-L Melanoma Growth by Compound I(d).

Compound I(d) was evaluated in an in vivo tumor growth inhibition assay. Athymic nude mice, of approximately 13 weeks in age, received injections, subcutaneously in the left flank, of 5×10⁶ M21-L cells suspended in 50 μl of unsupplemented DMEM. Tumors were allowed to grow to approximately 88 mm³. At this time, mice were assigned to one of five groups for treatment with 3, 10, 30 or 90 mg/kg/day of Compound I(d), or control. Tumor volume was then further monitored for each animal over the 35 day experiment.

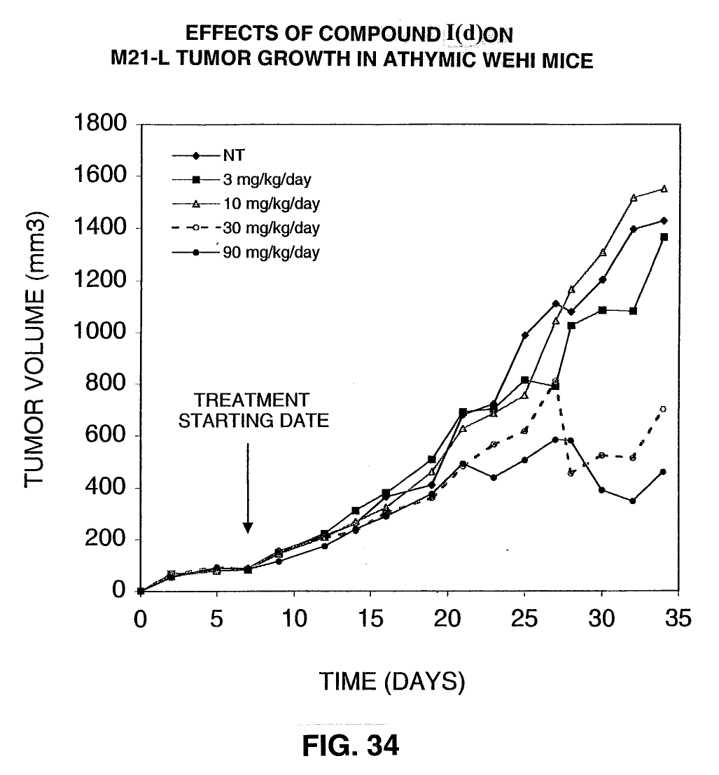

Mean tumor volume in all animals grew uniformly during the first seven days following M21-L tumor cell injections. After treatment with either 30 or 90 mg/kg/day of Compound I(d), tumor growth above 500-700 mm³ in mice was inhibited, as contrasted with an average tumor volume of 1450 mm³ with low dosage treatments or control. The results shown in FIG. 34 indicated about 65% inhibition of M21-L melanoma tumor growth at a dosage of about 90 mg/kg, and about 50% inhibition at a dosage of about 30 mg/kg of Compound I(d).

### EXAMPLE 14

Inhibition of M21-L Melanoma Growth by Compound I(l) on a CAM

In addition, Compound I(l) was evaluated in the M21-L melanoma tumor growth inhibition assay on a CAM as described in Example 7, above. A level of inhibition of about 40% was observed at a dosage level of about 100 μg/egg.

Thus, the aforementioned Examples demonstrate that integrin αᵥβ₃ plays a key role in angiogenesis induced by a variety of stimuli and as such αᵥβ₃ is a valuable therapeutic target with the αᵥβ₃ antagonists of this invention for diseases characterized by neovascularization.

The foregoing written specification is considered to be sufficient to enable one skilled in the art to practice the invention. Indeed, various modifications of the invention in addition to those shown and described herein will be apparent to those skilled in the art from the foregoing description and fall within the scope of-the appended claims.

We claim:

1. A method for inhibiting angiogenesis in a tissue comprising administering to the tissue a composition comprising an angiogenesis-inhibiting amount of an organic peptidomimetic αᵥβ₃ antagonist.

2. A method for inhibiting angiogenesis in a tissue and comprising administering to the tissue, in a pharmacologically acceptable carrier, an angiogenesis-inhibiting amount of an organic peptidomimetic αᵥβ₃ antagonist molecule that has a basic group and an acidic group spaced from one another at a distance in the range of about 10 Angstroms to about 100 Angstroms.

3. The method of claim 2 wherein the antagonist molecule is an organic peptidomimetic compound represented by the formula (I): 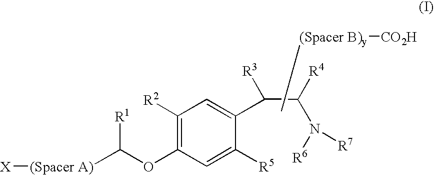
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and lo-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂)alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C(OZ²—), —NHCH₂CH₂—, —NHC(O)—, —NHC(O)CH₂—, and —NHC(O)CH₂CH₂—;
Spacer B is a radical selected from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R⁴; y is 0 or 1, with the proviso that when R⁵ and R⁶ form a covalent bond, y is 1.

4. The method of claim 3 wherein the organic peptidomimetic compound is represented by the formula:

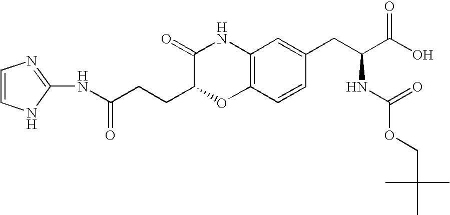

5. The method of claim 3 wherein the organic peptidomimetic compound is represented by the formula:

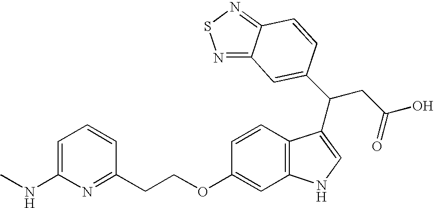

6. The method of claim 3 wherein the organic peptidomimetic compound is represented by the formula:

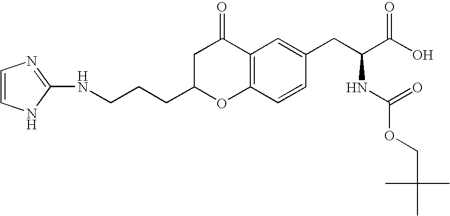

7. The method of claim 3 wherein the organic peptidomimetic compound is represented by the formula:

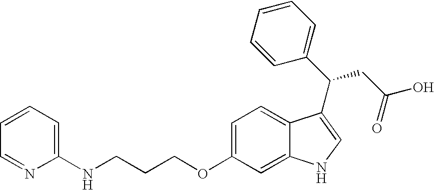

8. The method of claim 3 wherein the organic peptidomimetic compound is represented by the formula:

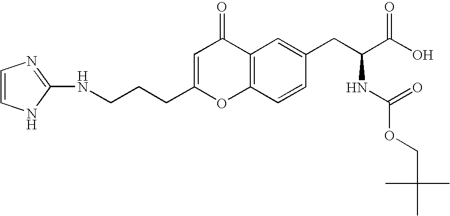

9. The method of claim 3 wherein the organic peptidomimetic compound is represented by the formula:

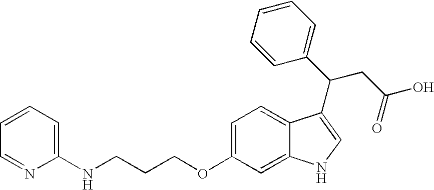

10. The method of claim 3 wherein the organic peptidomimetic compound is represented by the formula:

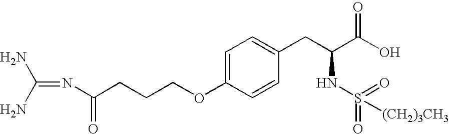

11. The method of claim 3 wherein the organic peptidomimetic compound is represented by the formula:

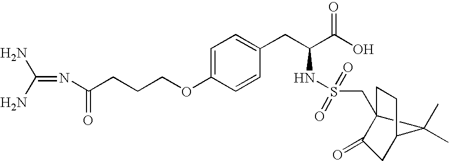

12. The method of claim 3 wherein the organic peptidomimetic compound is represented by the formula:

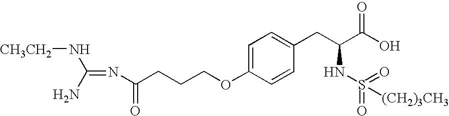

13. The method of claim 3 wherein the organic peptidomimetic compound is represented by the formula:

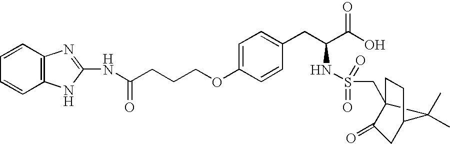

14. The method of claim 3 wherein the organic peptidomimetic compound is represented by the formula

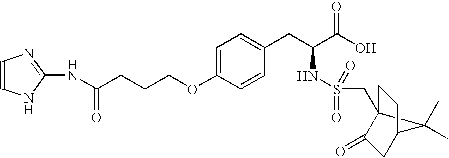

15. The method of claim 3 wherein the organic peptidomimetic compound is represented by the formula

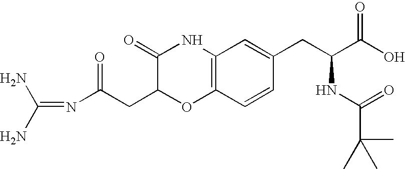

16. The method of claim 3 wherein the organic peptidomimetic compound is represented by the formula:

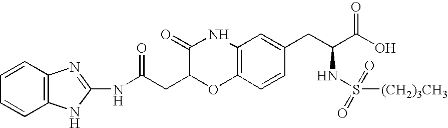

17. The method of claim 3 wherein the organic peptidomimetic compound is represented by the formula:

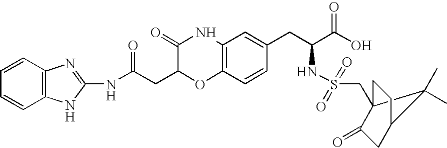

18. The method of claim 3 wherein the organic peptidomimetic compound is represented by the formula:

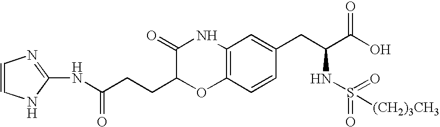

19. The method of claim 3 wherein the organic peptidomimetic compound is represented by the formula:

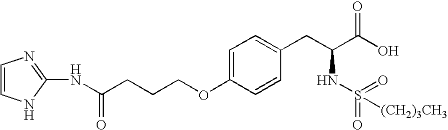

20. The method of claim 3 wherein the organic peptidomimetic compound is represented by the formula:

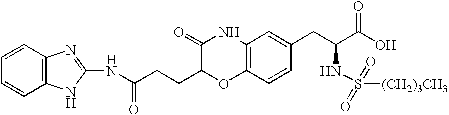

21. The method of claim 3 wherein the organic peptidomimetic compound is represented by the formula:

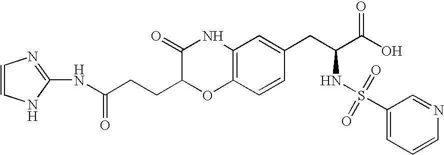

22. The method of claim 1 wherein the angiogenesis is inflamed tissue angiogenesis and the organic peptidomimetic αᵥβ₃ antagonist is administered to inflamed tissue.

23. The method of claim 22 wherein the organic peptidomimetic αᵥβ₃ antagonist is administered to arthritic tissue.

24. The method of claim 23 wherein the organic peptidomimetic αᵥβ₃ antagonist is administered to arthritic tissue present in a mammal with rheumatoid arthritis.

25. The method of claim 1 wherein the angiogenesis is retinal angiogenesis and the organic peptidomimetic αᵥβ₃ antagonist is administered to retinal tissue of a patient with diabetic retinopathy.

26. The method of claim 1 wherein the antiogenesis is tumor angiogenesis and the organic peptidomimetic αᵥβ₃ antagonist is administered to a solid tumor or a solid tumor metastasis.

27. The method of claim 26 wherein the organic peptidomimetic αᵥβ₃ antagonist is administered intravenously, transdermally, intrasynovially, intramuscularly, or orally.

28. The method of claim 26 wherein the organic peptidomimetic αᵥβ₃ antagonist is administered in conjunction with chemotherapy.

29. The method of claim 26 wherein the organic peptidomimetic αᵥβ₃ antagonist is administered intravenously as a single dose.

30. A method of inducing solid tumor tissue regression in a patient and comprising administering to the patient an organic peptidomimetic αᵥβ₃ antagonist in an amount sufficient to inhibit neovascularization of a solid tumor tissue.

31. The method of claim 30 wherein the organic peptidomimetic αᵥβ₃ antagonist is a compound represented by the formula (I): 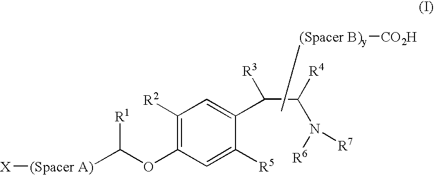
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂) alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O)CH₂—, and —NHC(O)CH₂CH₂—;
Spacer B is a radical selected from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R^{4;} y is 0 or 1, with the proviso that when R⁵ and R⁶ form a covalent bond, y is 1.

32. A method of inhibiting solid tumor tissue growth undergoing neovascularization in a patient and comprising administering to the patient an organic peptidomimetic αᵥβ₃ antagonist in an amount sufficient to inhibit solid tumor tissue growth.

33. The method of claim 32 wherein the organic peptidomimetic αᵥβ₃ antagonist is a compound represented by the formula (I): 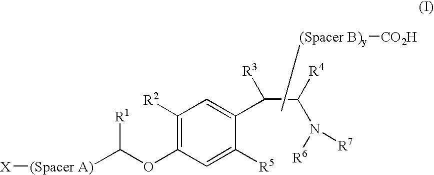
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂)alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C (OZ²)—, —NHCH₂CH₂—, —NHC(O)—, —NHC(O)CH₂—, and —NHC(O)CH₂CH₂—;
Spacer B is a radical selected, from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R⁴; y is 0 or 1, with the proviso that when R⁵ and R⁶ form a covalent bond, y is 1.

34. A method for treating a patient with inflamed tissue in which neovascularization is occurring and comprising administering to the patient a therapeutically effective amount of an organic peptidomimetic αᵥβ₃ antagonist in a physiologically compatible carrier.

35. The method of claim 34 wherein the organic peptidomimetic αᵥβ₃ antagonist is a compound represented by the formula (I): 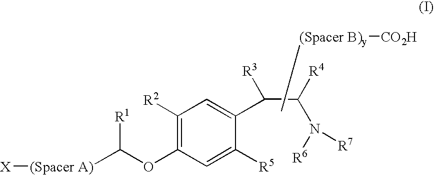
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂)alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O)CH₂—, and —NHC(O)CH₂CH₂—;
Spacer B is a radical selected from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R⁴; y is 0 or 1, with the proviso that when R⁵ and R⁶ form a covalent bond, y is 1.

36. A method for treating a patient in which neovascularization is occurring in retinal tissue and comprising administering to the patient a neovascularization-inhibiting amount of an organic peptidomimetic αᵥβ₃ antagonist.

37. The method of claim 36 wherein the organic peptidomimetic αᵥβ₃ antagonist is a compound represented by the formula (I): 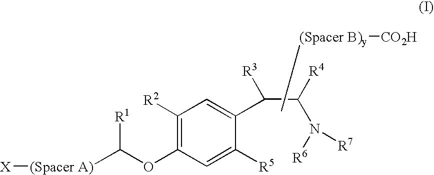
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂)alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O) CH₂—, and —NHC(O)CH₂CH₂—;
Spacer B is a radical selected from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R⁴; y is 0 or 1, with the proviso that when R⁵ and R⁶ form a covalent bond, y is 1.

38. A method for treating a patient for restenosis in a tissue wherein smooth muscle cell migration occurs following angioplasty, the method comprising administering to the patient an organic peptidomimetic αᵥβ₃ antagonist in an amount sufficient to inhibit smooth muscle cell migration.

39. The method of claim 38 wherein the organic peptidomimetic αᵥβ₃ antagonist is a compound represented by the formula (I): 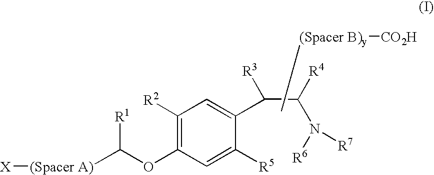
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected -from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂)alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O)CH₂—, and —NHC(O)CH₂CH₂—;
Spacer B is a radical selected from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R⁴; y is 0 or 1, with the proviso that when R⁵ and R⁶ form a covalent bond, y is 1.

40. A method of reducing blood supply to a tissue required to support new growth of the tissue in a patient, the method comprising administering to the patient an organic peptidomimetic αᵥβ₃ antagonist in an amount sufficient to reduce the blood supply to the tissue.

41. The method of claim 40 wherein the organic peptidomimetic αᵥβ₃ antagonist is a compound represented by the formula (I): 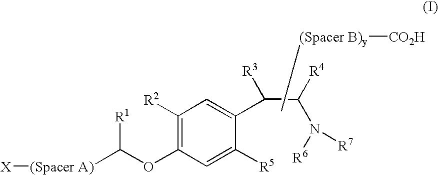
wherein
R¹ and R² are both H or together form a radical selected from the group consisting of —CH₂—C(O)—, ═C—C(O)—, and —C(O)—NH—;
R³ and R⁴ are both H or together form a covalent bond;
R⁵ and R⁶ are both H or, when R³ and R⁴ together form a covalent bond, R⁵ and R⁶ together form a covalent bond;
R⁷ is selected from the group consisting of tert-butoxycarbonyl, neo-pentyloxycarbonyl, 2-ethanesulfonyl, 3-propanesulfonyl, 4-butanesulfonyl, 3-pyridinesulfonyl, and 10-camphoresulfonyl; with the proviso that when R³ and R⁴ together form a covalent bond, R⁷ is H;
X is selected from the group consisting of 2-imidazolyl, 2-benzimidazolyl, N-guanidyl, N-(C₁-C₂)alkyl-substituted guanidyl, 2-pyridyl, 4-carbonimidophenyl, and 6-(2-methylaminopyridyl);
Spacer A is a radical selected from the group consisting of —CH₂—, —CH₂CH₂—, —CH₂CH₂CH₂—, —NZ¹CH₂C(OZ²)—, —NHCH₂CH₂—, —NHC(O)—, —NHC(O)CH₂—, and —NHC(O)CH₂CH₂—;
Spacer B is a radical selected from the group consisting of —CH₂—, —CH₂CH₂—, and —CH(R⁸)CH₂—;
Z¹ and Z² are both covalent bonds to a bridging carbonyl group forming a cyclic urethane;
R⁸ is phenyl or 5-benzo-2, 1, 3-thiadiazolyl; and
Spacer B is covalently bonded to either of the carbon atoms bearing substituents R³ and R⁴; y is 0 or 1, with the proviso that when R⁵ and R⁶ form a covalent bond, y is 1.
